(19) United States
(12) Patent Application Publication
(10) Pub. No.: US 2011/0230493 A1
(43) Pub. Date: Sep. 22, 2011

(54) 1-OXA-8-Azaspiro [4,5] Decabe-8-Carboxamide Compounds as FAAH Inhibitors

(75) Inventors: Scott Allen Long, Ballwin, MO (US); Marvin Jay Meyers, Wentzville, MO (US); Matthew James Pelc, Kenosha, WI (US); Barbara Ann Schweitzer, Webster Groves, MO (US); Lijuan Jane Wang, Wildwood, MO (US); Atli Thorarensen, Stow, MA (US)

(73) Assignee: Pfizer Inc.

(21) Appl. No.: 13/129,824

(22) PCT Filed: Nov. 9, 2009
(86) PCT No.: PCT/IB2009/054969

§ 371 Date: May 18, 2011

Related U.S. Application Data
(60) Provisional application No. 61/116,691, filed on Nov. 21, 2008.

(51) Int. Cl. A61K 31/501; A61K 31/438; A61P 9/00; C07D 491/107; A61P 19/02; A61P 3/10; A61P 29/00; A61P 9/12; A61P 11/06; A61P 1/00; A61P 25/00; A61P 35/04

(52) U.S. Cl. 514/252.03; 514/278; 544/238; 546/16

(57) ABSTRACT

Provided herein are 1-oxa-8-azaspiro[4.5]decane-8-carboxamide compounds of formula I

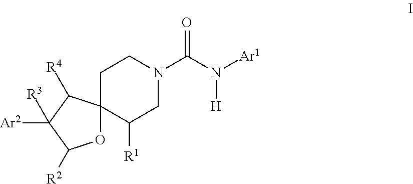

wherein Ar¹, Ar², R¹, R², R³ and R⁴ are as defined herein and the pharmaceutically acceptable salts of such compounds useful in treating diseases or conditions associated with fatty acid amide hydrolase (FAAH) activity, conditions including acute pain, chronic pain, neuropathic pain, nociceptive pain, inflammatory pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease.

### FIELD OF THE INVENTION

The present invention relates to 1-oxa-8-azaspiro[4.5]decane-8-carboxamide compounds and the pharmaceutically acceptable salts of such compounds. The invention also relates to the processes for the preparation of the compounds, intermediates used in their preparation, compositions containing the compounds, and the uses of the compounds in treating diseases or conditions associated with fatty acid amide hydrolase (FAAH) activity.

### BACKGROUND OF THE INVENTION

Fatty acid amides represent a family of bioactive lipids with diverse cellular and physiological effects. Fatty acid amides are hydrolyzed to their corresponding fatty acids by an enzyme known as fatty acid amide hydrolase (FAAH). FAAH is a mammalian integral membrane serine hydrolase responsible for the hydrolysis of a number of primary and secondary fatty acid amides, including the neuromodulatory compounds anandamide and oleamide. Anandamide (arachidonoyl ethanolamide) has been shown to possess cannabinoid-like analgesic properties and is released by stimulated neurons. The effects and endogenous levels of anandamide increase with pain stimulation, implying its role in suppressing pain neurotransmission and behavioral analgesia. Supporting this, FAAH inhibitors that elevate brain anandamide levels have demonstrated efficacy in animal models of pain, inflammation, anxiety, and depression. Lichtman, A. H. et al. (2004), J. Pharmacol. Exp. Ther. 311, 441-448; Jayamanne, A. et al. (2006), Br. J. Pharmacol. 147, 281-288; Kathuria, S. et al. (2003), Nature Med., 9, 76-81; Piomelli D. et al. (2005), Proc. Natl. Acad. Sci. 102, 18620-18625.

Further recent reviews on this subject are as follows:
Ahn, Kay; McKinney, Michele K.; Cravatt, Benjamin F, Chemical Reviews (Washington, D.C., United States) (2008), 108(5), 1687-1707;
Ahn, Kay; Johnson, Douglas S.; Cravatt, Benjamin F, Expert Opin. Drug Discov. (2009) 4(7), pp 763-784;
M Seierstad and J. G. Breitenbucher, Discovery and Development of Fatty Acid Amide Hydrolase (FAAH) Inhibitors, J. Med. Chem. XXXX, vol. xxx, no. xx, Published on Web Nov. 5, 2008.

WO 2006/085196 teaches a method for measuring activity of an ammonia-generating enzyme, such as FAAH. WO 2006/067613 teaches compositions and methods for expression and purification of FAAH. WO 2008/047229 teaches biaryl ether urea compounds useful for treating FAAH-mediated conditions. WO2006/074025 concerns piperazinyl and piperidinyl ureas as FAAH modulators.

There remains a need for new compounds that are inhibitors of FAAH and, therefore, are useful in the treatment of a wide range of disorders, including pain.

### SUMMARY OF THE INVENTION

Provided herein are compounds of the Formula I:

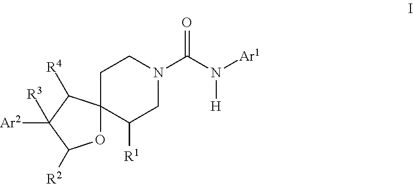

wherein Ar¹, Ar², R¹, R², R³ and R⁴ are as defined below, or a pharmaceutically acceptable salt thereof.

Also provided are pharmaceutical compositions comprising a therapeutically effective amount of a compound of Formula I, or a pharmaceutically acceptable salt thereof, and a pharmaceutically acceptable carrier. Further provided herein are methods of using a compound of Formula I, or a pharmaceutically acceptable salt thereof, in treating FAAH-mediated diseases or conditions.

### DETAILED DESCRIPTION

Provided herein are compounds of Formula I:

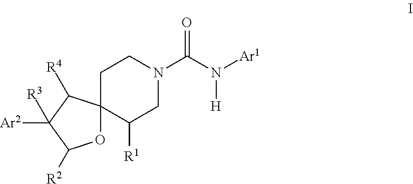

wherein:
Ar¹ is selected from:

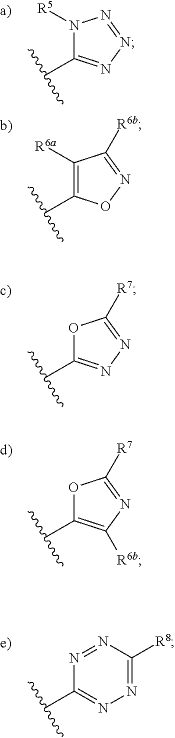

f) benzoisoxazole optionally substituted by 1 to 3 halo, C₁-C₃ alkyl, C₁-C₃ alkoxy, C₁-C₃ haloalkyl or C₁-C₃ haloalkoxy substituents; or
g) pyridine, pyridazine, pyrimidine, or pyrazine; wherein the pyridine, pyridazine, pyrimidine, or pyrazine is optionally substituted by 1 to 3 halo, C₁-C₃ alkyl, —(CH₂)ₙ—(C₃-C₆ cycloalkyl), C₁-C₃ alkoxy, C₁-C₃ haloalkyl or C₁-C₃ haloalkoxy substituents;
   Ar² is selected from:
a) phenyl optionally substituted by 1 to 5 halo, C₁-C₆ alkyl, —(CH₂)ₙ—, —(C₃-C₆ cycloalkyl), C₁-C₆ alkoxy, —(CH₂)ₙ—(C₃-C₆ cycloalkoxy), C₁-C₆ haloalkyl, C₁-C₆ haloalkoxy, —O—CH₂—CH₂—O—(C₁-C₆ alkyl), or —O—CH₂—CH₂—O—(C₁-C₆ haloalkyl) substituents; wherein the phenyl is further optionally substituted by a substituent of the formulae —R⁹, —O—R⁹, —O—(CH₂)ₚ—R⁹, or —(CH₂)ₚ—O—R⁹;
b) oxazole, isoxazole, thiazole, isothiazole, oxadiazole, or thiadiazole substituted by a substituent of the formulae —(CH₂)ₙ—R⁹, —(CH₂)ₘ—O—R⁹, or —(CH₂)ₚ—O—(CH₂)ₚ—R⁹;
c) a heterocycle of the formula:

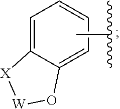

wherein X is CH₂ or O, and W is (CH₂)ₘ or CF₂; or
d) naphthyl, quinolinyl or isoquinolinyl optionally substituted by 1 to 3 halo, C₁-C₃ alkyl, C₁-C₃ alkoxy, C₁-C₃ haloalkyl or C₁-C₃ haloalkoxy substituents;
   R¹ is hydrogen, F, or CH₃;
   R² is hydrogen or CH₃;
   R³ is hydrogen, CH₃, —O—CH₃, OH, CN, or F;
   R⁴ is hydrogen, F, or CH₃;
   R⁵ is hydrogen, C₁-C₆ alkyl, —(CH₂)ₙ—(C₃-C₆ cycloalkyl), or C₁-C₆ haloalkyl;
   R^{6a} is C₁-C₃ alkyl;
   R^{6b} is hydrogen, C₁-C₆ alkyl, or C₁-C₃ haloalkyl;
   R⁷ is C₁-C₃ alkyl, —(CH₂)ₙ—(C₃-C₆ cycloalkyl), R⁹, or —CH₂—O—R⁹;
   R⁸ is phenyl optionally substituted by from 1 to 3 halo, C₁-C₃ alkyl, C₁-C₃ alkoxy, C₁-C₃ haloalkyl or C₁-C₃ haloalkoxy substituents;
   R⁹ is selected from phenyl, naphthyl, or heteroaryl; wherein R⁹ is optionally substituted by from 1 to 3 halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, or C₁-C₃ haloalkoxy substituents;
   m is 1, 2 or 3; n is 0, 1, 2, 3 or 4; and p is 1 or 2;
   or a pharmaceutically acceptable salt thereof.

Further provided are compounds within the groups of compounds described above wherein Ar² is selected from:
a) phenyl optionally substituted by from 1 to 3 substituents selected from F, Cl, Br, methyl, ethyl, CF₃, OCH₃, or OCF₃; wherein the phenyl may also be further substituted by a substituent of the formulae —R⁹, —O—R⁹ or —O—CH₂—R⁹;
b) thiazole or oxadiazole substituted by a substituent of the formulae —R⁹; or
   R¹, R², and R⁴ are hydrogen;
   R³ is hydrogen or F;
   R⁵, R^{6a}, and R^{6b} are methyl;
   R⁹ is phenyl, pyridine, or pyrimidine; wherein the R⁹ ring is optionally substituted by from 1 to 3 substituents selected from F, Cl, Br, CF₃, or OCF₃; or a pharmaceutically acceptable salt thereof.

Within each of the groups of compounds, and salts thereof, described herein are subgroups in which the variables R¹, R², R³, and R⁴ are each hydrogen. It is understood that the optional substituents on the Ar¹ and Ar² groups described herein are selected independently and each ring so described may contain the number of listed substituents that are the same or different from each other.

Also provided within each of the groups of compounds described herein is a subset of compounds, including pharmaceutically acceptable salts thereof, wherein R⁹, when present, is phenyl, pyridine or pyrimidine, each optionally by from 1 to 3 substituents selected from halo, C₁-C₃ alkyl, —(CH₂)ₙ—, —(C₃-C₆ cycloalkyl), C₁-C₃ alkoxy, —(CH₂)ₙ—(C₃-C₆ cycloalkoxy), C₁-C₃ haloalkyl or C₁-C₃ haloalkoxy; and n is 0, 1, 2, 3 or 4. Within each of these groups is a further subset wherein R⁹ is optionally substituted by 1 to 3 substituents selected from F, Cl, Br, CF₃, or OCF₃; or a pharmaceutically acceptable salt thereof.

Further provided within each of the groups of compounds described herein are compounds wherein:

Ar¹ is selected from:

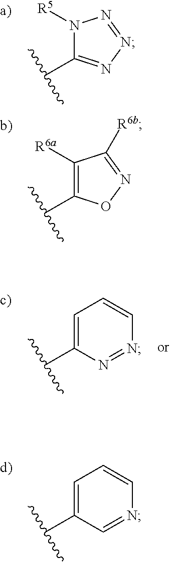

Ar² is selected from formulae, wherein R, R′, R″ and Z in each case are as defined under each formula:

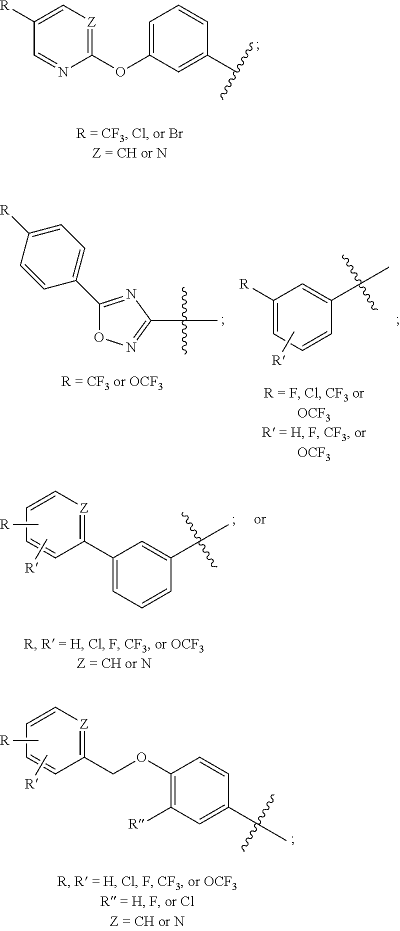

R¹, R², and R⁴ are H; R³ is H or F; and R⁵, R^{6a}, and R^{6b} are methyl; or a pharmaceutically acceptable salt thereof.

Also provided within each of the groups of compounds described herein are compounds wherein, when Ar² is oxadiazole, the oxadiazole is 1,2,4-oxadiazole; or a pharmaceutically acceptable salt thereof. Also provided within each of the groups of compounds described herein are compounds wherein, when Ar² is thiazole, the thiazole is 1,3-thiazole; or a pharmaceutically acceptable salt thereof. In each case the Ar² oxadiazole and thiazole groups may be optionally substituted as described herein.

In each of the groups described herein it is understood that, when a list of optional substituents is provided, each of the substituents is independently selected from the group of substituents.

The skilled person will realise that there are several possible quaternary carbon stereocentres (e.g. see “*” below) in the formula I depending on the substituents, and including possible stereocentres in the substituents, which therefore offer a variety of possible stereoisomers. It is to be understood that the formula I embraces all such stereoisomers, including specific enantiomers, racemic mixtures, diastereomeric mixtures etc.

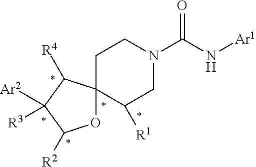

Preferable groups of compounds of formula I and their pharmaceutically acceptable salts are those wherein independently:

R¹ has the value of R¹ of any of the specific compounds mentioned below;
R² has the value of R² of any of the specific compounds mentioned below;
R³ has the value of R³ of any of the specific compounds mentioned below;
R⁴ has the value of R⁴ of any of the specific compounds mentioned below;
Ar¹ has the value of Ar¹ of any of the specific compounds mentioned below; and
Ar² has the value of Ar² of any of the specific compounds mentioned below.

The most preferable compounds of formula I and their pharmaceutically acceptable salts are the compounds specifically mentioned below and their pharmaceutically acceptable salts.

Also provided are pharmaceutical compositions comprising a therapeutically effective amount of a compound herein, or a pharmaceutically acceptable salt thereof, and a pharmaceutically acceptable carrier. Further provided herein are methods of treating FAAH-mediated diseases or conditions including acute pain, chronic pain, neuropathic pain, nociceptive pain, inflammatory pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease in a subject by administering to a subject in need thereof a therapeutically effective amount of one or more of the compounds herein, or a pharmaceutically acceptable salt thereof. Provided herein is also the use of a compound described herein, or a pharmaceutically acceptable salt thereof, in the manufacture of a medicament for the treatment of a FAAH-mediated disease or condition. Individual methods using a compound described herein, or a pharmaceutically acceptable salt thereof, in the manufacture of a medicament for the treatment of each of the individual diseases or conditions described herein are also provided.

This disclosure uses the definitions provided below. Some chemical formulae may include a dash (“-”) to indicate a bond between atoms or indicate a point of attachment. “Substituted” groups are those in which one or more hydrogen atoms have been replaced with one or more non-hydrogen atoms or groups, the “substituents”. “Alkyl” refers to straight chain or branched chain saturated hydrocarbon groups, generally having a specified number of carbon atoms (i.e., C₁-C₆alkyl). “Alkoxy” refers to alkyl-O— groups wherein the alkyl portions may be straight chain or branched, such as methoxy, ethoxy, n-propoxy, and i-propoxy groups. “Halo,” or “halogen” may be used interchangeably, and are fluoro, chloro, bromo, and iodo. The terms “haloalkyl”, “haloalkoxy” or “—O-haloalkyl” refer, respectively, to alkyl or alkoxy groups substituted by one or more halogens. Examples include —CF₃, —CH₂—CF₃, —CF₂—CF₃, —O—CF₃, and —OCH₂—CF₃. “Cycloalkyl” refers to saturated monocyclic and bicyclic hydrocarbon rings, generally having a specified number of carbon atoms that comprise the ring (i.e. C₃-C₆ cycloalkyl), optionally including one or more substituents. Examples of monocyclic cycloalkyl groups include cyclopropyl, cyclobutyl, cyclopentyl, cyclohexyl, and the like. “Cycloalkoxy” or “−O-cycloalkyl” refer to cycloalkyl groups attached through an oxygen atom, such as cyclopropoxy, cyclobutoxy, cyclopentoxy, and cyclohexoxy groups. The abbreviations R.T., RT, r.t. or rt refer to “room temperature”.

“Heteroaryl” and “heteroarylene” refer to monovalent or divalent aromatic groups, respectively, containing from 1 to 4 ring heteroatoms selected from O, S or N. Examples of monocyclic heteroaryl groups include pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, and the like. Heteroaryl and heteroarylene groups also include bicyclic groups, including fused ring systems wherein at least one ring is aromatic. Examples of bicyclic heteroaryl groups include benzofuranyl, benzothiopheneyl, indolyl, benzoxazolyl, benzodioxazolyl, benzimidazolyl, indazolyl, benzotriazolyl, benzothiofuranyl, benzothiazolyl, benzotriazolyl, benzotetrazolyl, benzoisoxazolyl, benzoisothiazolyl, benzoimidazolinyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, purinyl, indolizinyl, imidazo[1,2-a]pyridinyl, imidazo[1,5-a]pyridinyl, pyrazolo[1,5-a]pyridinyl, pyrrolo[1,2-b]pyridinyl, and imidazo[1,2-c]pyridinyl. Other examples include quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, isobenzofuranyl, isochromanyl, pteridinyl, oxazolo[5,4-c]pyridinyl, oxazolo[4,5-c]pyridinyl, oxazolo[5,4-b]pyridinyl, oxazolo[4,5-b]pyridinyl, isoxazolopyridinyl, thiazolylpyridinyl, oxazolopyrimidinyl, and the like.

“Subject” refers to a mammal, including humans, as well as companion animals, such as dogs and cats, and commercial or farm mammals, such as hogs, cattle, horses, goats, sheep, rabbits, etc. “Treating” refers to reversing, alleviating, inhibiting the progress of a disorder or condition to which such term applies, or to reversing, alleviating, inhibiting the progress of, or preventing one or more symptoms of such disorder or condition. “Therapeutically effective amount” refers to the quantity of a compound that may be used for treating a subject, which amount may depend on the subject's weight and age and the route of administration, among other things. “Excipient” or “adjuvant” refers to any substance in a pharmaceutical formulation that is not an active pharmaceutical ingredient (API). “Pharmaceutical composition” refers to a combination of one or more drug substances and one or more excipients. “Drug product,” “pharmaceutical dosage form,” “dosage form,” “final dosage form” and the like, refer to a pharmaceutical composition that is administered to a subject in need of treatment and generally may be in the form of tablets, capsules, liquid solutions, suspensions, patches, films, and the like.

Pharmaceutically acceptable carriers are understood to be agents, other than the active pharmacological ingredients, used in the preparation, maintenance or delivery of pharmaceutical formulations. Non-limiting examples of classes of pharmaceutically acceptable carriers include fillers, binders, disintegrants, bulking agents, lubricants, colorants, solubilizing agents, adjuvants, excipients, coating agents, glidants, diluents, emulsifiers, solvents, surfactants, emollients, adhesives, anti-adherents, wetting agents, sweeteners, flavoring agents, antioxidants, alkalizing agents, acidifiers, buffers, adsorbents, stabilizing agents, suspending agents, preservatives, plasticizers, nutrients, bioadhesives, extended and controlled release agents, stiffening agents, humectants, penetration enhancers, chelating agents, and the like.

The compounds herein and the pharmaceutically acceptable salts thereof, which includes those of Formula I, may be used to treat acute pain, chronic pain, neuropathic pain, nociceptive pain, inflammatory pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.

Physiological pain is a protective mechanism designed to warn of danger from potentially injurious stimuli from the external environment and may be classified as acute or chronic. Acute pain begins suddenly, is short-lived (usually 12 weeks or less), is usually associated with a specific cause, such as a specific injury, and is often sharp and severe. Acute pain does not generally result in persistent psychological response. Chronic pain is long-term pain, typically lasting for more than 3 months and leading to psychological and emotional problems. Examples of chronic pain are neuropathic pain (e.g. painful diabetic neuropathy, postherpetic neuralgia), carpal tunnel syndrome and back, headache, cancer, arthritic and chronic post-surgical pain.

Clinical pain is present when discomfort and abnormal sensitivity feature among the patient's symptoms, including 1) spontaneous pain which may be dull, burning, or stabbing; 2) exaggerated pain responses to noxious stimuli (hyperalgesia); and 3) pain produced by normally innocuous stimuli (allodynia). Although patients suffering from various forms of acute and chronic pain may have similar symptoms, the underlying mechanisms may be different and require different treatment strategies. Pain can also be divided into different subtypes according to differing pathophysiology, including nociceptive, inflammatory and neuropathic pain. Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is defined as pain initiated or caused by a primary lesion or dysfunction in the nervous system. Nerve damage can be caused by trauma and disease and the term ‘neuropathic pain’ encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life. The symptoms of neuropathic pain include spontaneous pain, which can be continuous, and paroxysmal or abnormal evoked pain, such as hyperalgesia (increased sensitivity to a noxious stimulus) and allodynia (sensitivity to a normally innocuous stimulus).

Another type of inflammatory pain is visceral pain which includes pain associated with inflammatory bowel disease (IBD). Visceral pain is pain associated with the viscera, which encompass the organs of the abdominal cavity, including the sex organs, spleen and part of the digestive system. Visceral pain can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Visceral pain includes that associated with dysmenorrhea, cystitis, pancreatitis and pelvic pain.

Some types of pain have multiple etiologies and thus can be classified in more than one area, e.g. back pain and cancer pain have both nociceptive and neuropathic components. Other types of pain include pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome and temporomandibular myofascial pain.

As described above, the compounds herein, and the pharmaceutically acceptable salts thereof, may be used to treat CNS disorders, including schizophrenia and other psychotic disorders, mood disorders, anxiety disorders, sleep disorders, and cognitive disorders, such as delirium, dementia, and amnestic disorders. The standards for diagnosis of these disorders may be found in the American Psychiatric Association's Diagnostic and Statistical Manual of Mental Disorders (4th ed., 2000), commonly referred to as the DSM Manual.

For the purposes of this disclosure, schizophrenia and other psychotic disorders include schizophreniform disorder, schizoaffective disorder, delusional disorder, brief psychotic disorder, shared psychotic disorder, psychotic disorder due to general medical condition, and substance-induced psychotic disorder, as well as medication-induced movement disorders, such as neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor. Mood disorders include depressive disorders, such as major depressive disorder, dysthymic disorder, premenstrual dysphoric disorder, minor depressive disorder, recurrent brief depressive disorder, postpsychotic depressive disorder of schizophrenia, and major depressive episode with schizophrenia; bipolar disorders, such as bipolar I disorder, bipolar II disorder, cyclothymia, and bipolar disorder with schizophrenia; mood disorders due to general medical condition; and substance-induced mood disorders. Anxiety disorders include panic attack, agoraphobia, panic disorder without agoraphobia, agoraphobia without history of panic disorder, specific phobia, social phobia (social anxiety disorder), obsessive-compulsive disorder, posttraumatic stress disorder, acute stress disorder, generalized anxiety disorder, anxiety disorder due to general medical condition, substance-induced anxiety disorder, and mixed anxiety-depressive disorder. Sleep disorders include primary sleep disorders, such as dyssomnias (primary insomnia, primary hypersomnia, narcolepsy, breathing-related sleep disorder, circadian rhythm sleep disorder, sleep deprivation, restless legs syndrome, and periodic limb movements) and parasomnias (nightmare disorder, sleep terror disorder, sleepwalking disorder, rapid eye movement sleep behavior disorder, and sleep paralysis); sleep disorders related to another mental disorder, including insomnia related to schizophrenia, depressive disorders, or anxiety disorders, or hypersomnia associated with bipolar disorders; sleep disorders due to a general medical condition; and substance-induced sleep disorders, Delirium, dementia, and amnestic and other cognitive disorders, includes delirium due to a general medical condition, substance-induced delirium, and delirium due to multiple etiologies; dementia of the Alzheimer's type, vascular dementia, dementia due to general medical conditions, dementia due to human immunodeficiency virus disease, dementia due to head trauma, dementia due to Parkinson's disease, dementia due to Huntington's disease, dementia due to Pick's disease, dementia due to Creutzfeldt-Jakob disease, dementia due to other general medical conditions, substance-induced persisting dementia, dementia due to multiple etiologies; amnestic disorders due to a general medical condition, and substance-induced persisting amnestic disorder.

Substance-induced disorders refer to those resulting from the using, abusing, dependence on, or withdrawal from, one or more drugs or toxins, including alcohol, amphetamines or similarly acting sympathomimetics, caffeine, cannabis, cocaine, hallucinogens, inhalants, nicotine, opioids, phencyclidine or similarly acting arylcyclohexylamines, and sedatives, hypnotics, or anxiolytics, among others.

Urinary incontinence includes the involuntary or accidental loss of urine due to the inability to restrain or control urinary voiding. Urinary incontinence includes mixed urinary incontinence, nocturnal enuresis, overflow incontinence, stress incontinence, transient urinary incontinence, and urge incontinence.

The compounds described and specifically named herein may form pharmaceutically acceptable complexes, salts, solvates and hydrates. The salts include acid addition salts (including di-acids) and base salts.

Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, lactate, malate, maleate, malonate, mesylate, methylsulfate, naphthylate, 2-napsylate, nicotinate, nitrate, orotate, oxalate, almitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine.

Pharmaceutically acceptable salts may be prepared using various methods. For example, one may react a compound with an appropriate acid or base to give the desired salt. One may also react a precursor of the compound with an acid or base to remove an acid- or base-labile protecting group or to open a lactone or lactam group of the precursor. Additionally, one may convert a salt of the compound to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO). A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion. When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm. The compounds herein, and the pharmaceutically acceptable salts thereof, may also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together.

“Prodrugs” refer to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts.

“Metabolites” refer to compounds formed in vivo upon administration of pharmacologically active compounds. Examples include hydroxymethyl, hydroxy, secondary amino, primary amino, phenol, and carboxylic acid derivatives of compounds herein, and the pharmaceutically acceptable salts thereof having methyl, alkoxy, tertiary amino, secondary amino, phenyl, and amide groups, respectively. Geometrical (cis/trans) isomers may be separated by conventional techniques such as chromatography and fractional crystallization. “Tautomers” refer to structural isomers that are interconvertible via a low energy barrier. Tautomeric isomerism (tautomerism) may take the form of proton tautomerism in which the compound contains, for example, an imino, keto, or oxime group, or valence tautomerism in which the compound contains an aromatic moiety.

The compounds herein, and pharmaceutically acceptable salts thereof, can be administered as crystalline or amorphous forms, prodrugs, metabolites, hydrates, solvates, complexes, and tautomers thereof, as well as all isotopically-labelled compounds thereof. They may be administered alone or in combination with one another or with one or more other pharmacologically active compounds. Generally, one or more these compounds are administered as a pharmaceutical composition (a formulation) in association with one or more pharmaceutically acceptable excipients.

Also provided herein are pharmaceutical compositions comprising a therapeutically effective amount of a compound described herein, or a pharmaceutically acceptable salt thereof, and one or more pharmaceutically acceptable carriers and/or excipients. The compounds herein, and the pharmaceutically acceptable salts thereof, may be administered orally. Oral administration may involve swallowing in which case the compound enters the bloodstream via the gastrointestinal tract. Alternatively or additionally, oral administration may involve mucosal administration (e.g., buccal, sublingual, supralingual administration) such that the compound enters the bloodstream through the oral mucosa. Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, for example, from gelatin or hydroxypropyl methylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

The compounds herein, and the pharmaceutically acceptable salts thereof, may also be used in fast-dissolving, fast-disintegrating dosage forms such as those described in Liang and Chen, Expert Opinion in Therapeutic Patents, 11(6):981-986 (2001).

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate. Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet. Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet. Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant.

Consumable oral films for human or veterinary use are pliable water-soluble or water-swellable thin film dosage forms which may be rapidly dissolving or mucoadhesive. In addition to the active pharmaceutical agent, a typical film includes one or more film-forming polymers, binders, solvents, humectants, plasticizers, stabilizers or emulsifiers, viscosity-modifying agents, solvents and other ingredients. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film. Film dosage forms are typically prepared by evaporative drying of thin aqueous films coated onto a peelable backing support or paper, which may carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. Compounds herein, and the pharmaceutically acceptable salts thereof, may also be administered directly into the blood stream, muscle, or an internal organ of the subject. Suitable parenteral administrations include intravenous, intraarterial, intraperitoneal, intrathecal, intraventricular, intraurethral, intrasternal, intracranial, intramuscular, intrasynovial, and subcutaneous administration via needle injectors, microneedle injectors, needle-free injectors, and infusion devices.

The compounds herein, and the pharmaceutically acceptable salts thereof, may also be administered topically, intradermally, or transdermally to the skin or mucosa. Typical formulations for this purpose include gels, hydrogels, lotions, solutions, creams, liposomes, ointments, dusting powders, dressings, foams, films, skin patches, wafers, implants, sponges, fibers, bandages and microemulsions using carriers and methods known in the art.

The compounds herein, and the pharmaceutically acceptable salts thereof, may also be administered intranasally or by inhalation, typically in the form of a dry powder, an aerosol spray, or nasal drops. The active compounds may also be administered rectally or vaginally, e.g., in the form of a suppository, pessary, or enema.

In the case of dry powder inhalers and aerosols, the dosage unit is determined by means of a valve that delivers a metered amount. Units are typically arranged to administer a metered dose or “puff” containing from about 10 μg to about 1000 μg of API. The overall daily dose will typically range from about 100 μg to about 10 mg which may be administered in a single dose or as divided doses throughout the day.

As noted above, the compounds herein, and the pharmaceutically acceptable salts thereof, and their pharmaceutically active complexes, solvates and hydrates, may be combined with one another or with one or more other active pharmaceutically active compounds to treat various diseases, conditions and disorders. In such cases, the active compounds may be combined in a single dosage form as described above or may be provided in the form of a kit which is suitable for coadministration of the compositions.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these therapeutically effective dosages are based on an average human subject having a mass of about 60 kg to about 70 kg, the physician will be able to determine the appropriate dose for a patient (e.g., an infant) whose mass falls outside of this weight range.

The claimed and disclosed compounds may be combined with one or more other pharmacologically active compounds for the treatment of one or more related disorders, the pharmacologically active compounds can be selected from: 1) an opioid analgesic, e.g. morphine, fentanyl, codeine, etc.; 2) a nonsteroidal antiinflammatory drug (NSAID), e.g. acetaminophen, aspirin, diclofenac, etodolac, ibuprofen, naproxen, etc.; 3) a barbiturate sedative, e.g. pentobarbital; 4) a benzodiazepine having a sedative action, e.g. diazepam, lorazepam, etc.; 5) an H₁ antagonist having a sedative action, e.g. diphenhydramine; 6) a sedative such as glutethimide, meprobamate, methaqualone or dichloralphenazone; 7) a skeletal muscle relaxant, e.g. baclofen, carisoprodol, chlorzoxazone, cyclobenzaprine, methocarbamol or orphrenadine; 8) an NMDA receptor antagonist; 9) an alpha-adrenergic; 10) a tricyclic antidepressant, e.g. desipramine, imipramine, amitriptyline or nortriptyline; 11) an anticonvulsant, e.g. carbamazepine, lamotrigine, topiratmate or valproate; 12) a tachykinin (NK) antagonist, particularly an NK-3, NK-2 or NK-1 antagonist; 13) a muscarinic antagonist, e.g oxybutynin, tolterodine, etc.; 14) a COX-2 selective inhibitor, e.g. celecoxib, valdecoxib, etc.; 15) a coal-tar analgesic, in particular paracetamol; 16) a neuroleptic such as haloperidol, clozapine, olanzapine, risperidone, ziprasidone, or Miraxion®; 17) a vanilloid receptor (VR1; also known as transient receptor potential channel, TRPV1) agonist (e.g. resinferatoxin) or antagonist (e.g. capsazepine); 18) a beta-adrenergic such as propranolol; 19) a local anaesthetic such as mexiletine; 20) a corticosteroid such as dexamethasone; 21) a 5-HT receptor agonist or antagonist, particularly a 5-HT_{1B/1D} agonist such as eletriptan, sumatriptan, naratriptan, zolmitriptan or rizatriptan; 22) a 5-HT_{2A} receptor antagonist such as R(+)-alpha-(2,3-dimethoxy-phenyl)-1-[2-(4-fluorophenylethyl)]-4-piperidinemethanol (MDL-100907); 23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine, or a nicotine partial agonist such as varenicline; 24) Tramadol®; 25) a PDEV inhibitor; 26) an alpha-2-delta ligand such as gabapentin, pregabalin, 3-methylgabapentin, etc.; 27) a cannabinoid receptor (CB1, CB2) ligand, either agonist or antagonist such as rimonabant; 28) metabotropic glutamate subtype 1 receptor (nnGluR1) antagonist; 29) a serotonin reuptake inhibitor such as sertraline, sertraline metabolite demethylsertraline, fluoxetine, etc.; 30) a noradrenaline (norepinephrine) reuptake inhibitor, such as buproprion, buproprion metabolite hydroxybuproprion, especially a selective noradrenaline reuptake inhibitor such as reboxetine, in particular (S,S)-reboxetine; 31) a dual serotonin-noradrenaline reuptake inhibitor, such as venlafaxine, O-desmethylvenlafaxine, clomipramine, desmethylclomipramine, duloxetine, milnacipran and imipramine; 32) an inducible nitric oxide synthase (iNOS) inhibitor; 33) an acetylcholinesterase inhibitor such as donepezil; 34) a prostaglandin E₂ subtype 4 (EP4) antagonist; 35) a leukotriene B4 antagonist; 36) a 5-lipoxygenase inhibitor, such as zileuton; 37) a sodium channel blocker, such as lidocaine; 38) a 5-HT3 antagonist, such as ondansetron; or 39) anti-nerve growth factor (NGF) antibodies. It is understood that the pharmaceutical agents just mentioned may be administered in the manner and at the dosages known in the art.

The compounds described herein may be present as stereoisomers, such as enantiomers, diastereomers, and geometric isomers (cis/trans olefins). For example, the compounds generally comprise one or more asymmetric carbon atoms and can be present in the form of one or more stereoisomers (e.g., individual enantiomers and mixtures thereof).

The compounds described herein (including the precursor intermediates) can have one or more chiral centers and one or more alkenyl moieties. Where the synthesis yields a compound as a mixture of isomers (e.g., enantiomers, diastereomers, and/or geometric isomers), the desired isomer (or the desired enantiomerically-, diastereomerically-, or geometrically-enriched mixture) can be obtained using conventional chiral resolution methods including chromatography (such as HPLC) or supercritical fluid chromatography (SFC) on an asymmetric resin, such as Chiralcel OJ-H, Chiralpak AD-H, Chiralpak IA and Chiralpak AS-H brand chiral stationary phases available from Daicel Chemical Industries, Ltd, Japan, with a mobile phase typically comprising an alcohol (e.g., from about 10% to about 50% by volume) and carbon dioxide. Concentration of the eluate affords the isomerically enriched mixture, which may also be further derivatized.

The compounds herein, and the pharmaceutically acceptable salts thereof, may be generally prepared using the techniques described below. Starting materials and reagents may be obtained from commercial sources or may be prepared using literature methods unless otherwise specified. In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. A discussion of protecting group strategies can be seen in T. W. Greene and P. G. Wuts, Greene's Protective Groups in Organic Chemistry (4^{th} Ed., 2007) and P. Kocienski, Protective Groups (2000).

Generally, the chemical reactions described herein may be carried out using substantially stoichiometric amounts of reactants, though certain reactions may benefit from using an excess of one or more of the reactants. Also, many of the reactions disclosed may be carried out at about room temperature and ambient pressure, but depending on reaction kinetics, yields, and the like, some reactions may be run at elevated pressures or employ higher (e.g., reflux conditions) or lower (e.g., −70° C. to 0° C.) temperatures. Any reference to a stoichiometric range, a temperature range, a pH range, etc., whether or not expressly using the word “range,” also includes the indicated endpoints.

Many of the chemical reactions may also employ one or more compatible solvents, which may influence the reaction rate and yield. Depending on the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane); aromatic hydrocarbons (e.g., benzene, toluene, xylenes); halogenated hydrocarbons (e.g., methylene chloride (DCM), chloroform, carbon tetrachloride); aliphatic alcohols (e.g., methanol (MeOH), ethanol (EtOH), propan-1-ol, propan-2-ol (IPA), butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane (DME), 1,2-diethoxy-ethane, 1-methoxy-2-(2-methoxy-ethoxy)-ethane, 1-ethoxy-2-(2-ethoxy-ethoxy)-ethane, tetrahydrofuran (THF), 1,4-dioxane); ketones (e.g., acetone, methyl ethyl ketone (MEK)); esters (methyl acetate, ethyl acetate (EA or EtOAc); nitrogen-containing solvents (e.g., formamide, N,N-dimethyl formamide (DMF), acetonitrile, N-methyl-pyrrolidone (NMP), pyridine, quinoline, nitrobenzene); sulfur-containing solvents (e.g., carbon disulfide, dimethyl sulfoxide (DMSO), tetrahydro-thiophene-1,1,-dioxide); and phosphorus-containing solvents (e.g., hexamethylphosphoric triamide).

The compounds herein may be prepared as described below. In the reaction schemes and discussion herein, Ar¹, Ar², R¹, R², R³, and R⁴ are defined as above. Ar¹ and Ar² may also be substituted as defined above.

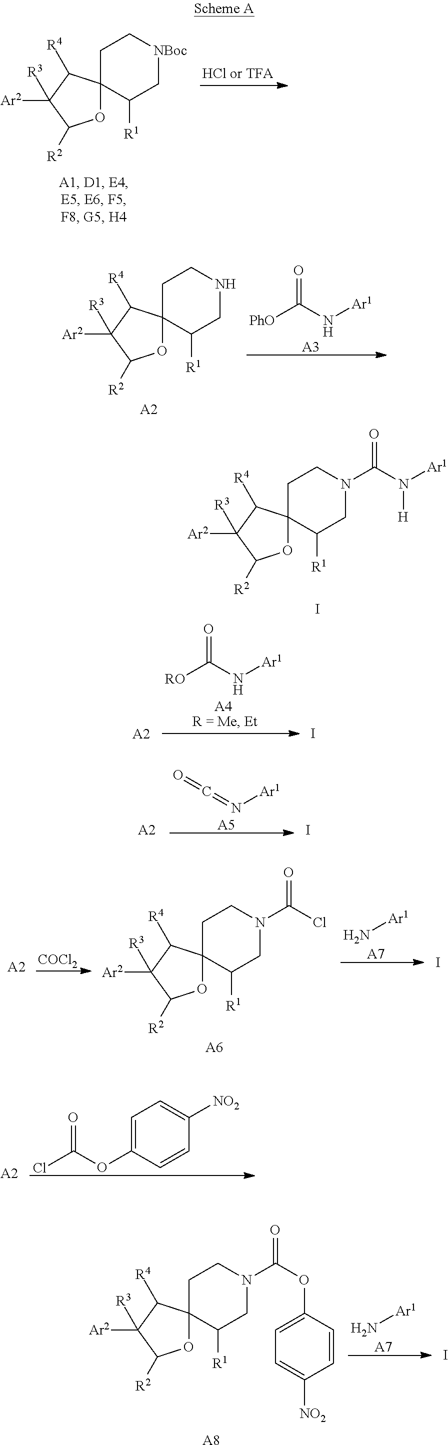

Compounds of Formula I can be prepared according to Scheme A. Compounds of formula A1, D1, E4, E5, E6, F5, F8, G5 and H4 can be deprotected using conventional methods (for example, using HCl/dioxane in dichloromethane, acetyl chloride in ethanol, or trifluoroacetic acid (TFA) in dichloromethane) to provide the corresponding compounds of formula A2 which can be isolated as the free base or as the corresponding salt (hydrochloride or trifluoroacetate). The reaction of a compound of formula A2 with a phenyl carbamate of formula A3 provides compounds of the Formula I. The reaction can be conducted in a polar aproptic solvent such as DMSO or acetonitrile. The temperature of the reaction may vary from about ambient temperature to about 60° C. The reaction can also be conducted using a trifluoroacetate or hydrochloride salt of the compound of formula A2 in the presence of a base such as triethylamine (TEA) or diisopropylethyl amine (DIEA). Alternatively, the reaction of a compound of formula A2 with a carbamate of formula A4 (R=Me or Et) under microwave irradiation may provide compounds of the Formula I. The reaction may be conducted in a solvent such as acetonitrile. The reaction may also be conducted using a trifluoroacetate or hydrochloride salt of the compound of formula A2 in the presence of a base such as TEA or DIEA. Furthermore, compounds of the Formula I may be prepared by reacting compounds of formula A2 with an isocyanate of formula A5. The reaction may be conducted in a solvent such as dichloromethane at ambient temperature. The reaction may also be conducted using a trifluoroacetate or hydrochloride salt of the compound of formula A2 in the presence of a base such as TEA or DIEA. Alternatively, compounds of formula A2 may be reacted with phosgene in the presence of a base such as TEA or DIEA and a solvent such as dichloromethane at about 0° C. to generate compounds of formula A6 which may be isolated as a crude material and reacted with aryl amines of formula A7 in the presence of a base such as TEA or DIEA and a catalyst such as 4-(dimethylamino)-pyridine (DMAP) in a suitable solvent such as acetonitrile, dichloromethane, and dichloroethane. The reaction temperature may vary from about ambient temperature to about 70° C. Alternatively, compounds of formula A2 may be reacted with 4-nitrophenyl chloroformate in the presence of a base such as aqueous sodium bicarbonate and a solvent such as dioxane at room temperature to generate compounds of formula A8 which may be isolated as a crude material, optionally purified, and reacted with aryl amines of formula A7 in the presence of a base such as sodium hydride in a suitable solvent such as DMF or DMA. The reaction temperature may vary from about ambient temperature to about 70° C.

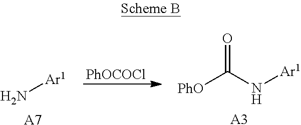

Scheme B illustrates a method for making phenyl carbamates of formula A3. Treatment of an aryl amine of formula A7 with phenyl chloroformate in a solvent such as THF, DCM, 1,4-dioxane, acetonitrile, DMF, or DMSO gives phenyl carbamates of formula A3 in a manner similar to that described in Synthesis, 1997, 1189-1194. The reaction may be performed in the presence of a base such as TEA, DIEA, 1,8-bis(dimethylamino)naphthalene (Proton Sponge®), and the like. The temperature of the reaction may vary from about 0° C. to reflux temperature of the solvent being used.

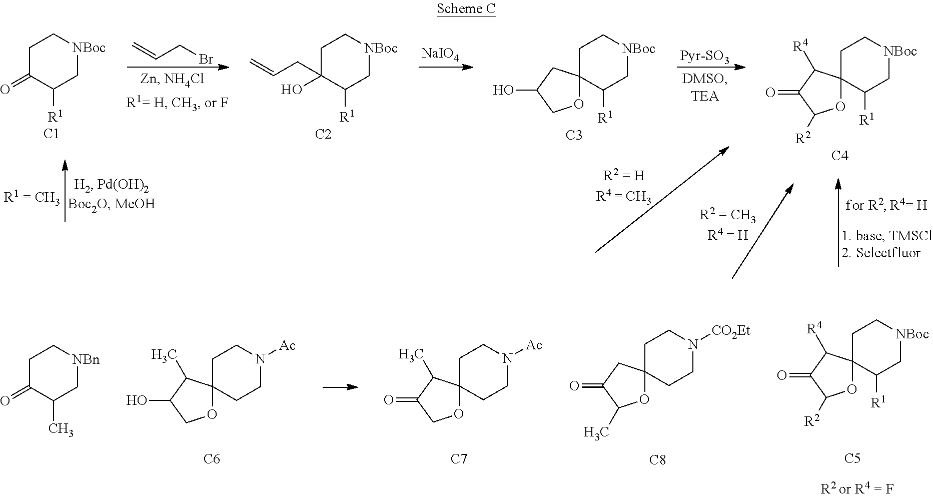

Ketone intermediates of formulae C4 and C5 can be prepared according to Scheme C. A compound of formula C1 (e.g., tert-butyl 4-oxopiperidine-1-carboxylate (CAS#79099-07-3), tert-butyl 3-fluoro-4-oxopiperidine-1-carboxylate (CAS#211108-50-8; van Niel et al. J. Med. Chem., 1999, 42, 2087-2104), or tert-butyl 3-methyl-4-oxopiperidine-1-carboxylate (CAS#181269-69-2) which can be prepared from 1-benzyl-3-methyl-piperidin-4-one (CAS#34737-89-8) as described by Luly et al. US 2005/0070549, Mar. 31, 2005) can be reacted with allyl bromide, Zn dust, and aqueous ammonium chloride in THF to give compounds of formula C2 (for R¹═H, CAS#203662-51-5). Compounds of formula C2 can be treated with sodium periodate in a mixed solvent system such as tert-butanol/water to give compounds of formula C3 (for R¹═H, CAS#240401-09-6). Compounds of formula C3 can be oxidized by conventional methods such as with pyridinium sulfur trioxide (Pyr-SO₃) and TEA in DMSO to give compounds of formula C4. Compounds of formula C4 may be further elaborated by lithiation with a strong base such as lithium diisopropylamide (LDA) or lithium hexamethyldisilazide (LHMDS or LiHMDS), trapped as the silyl enolate with trimethylsilylchloride (TMSCl), and reaction with a fluorinating agent such as Selectfluor® (CAS#140681-54-5) in a solvent such as THF to provide compounds of formula C5 (R² or R⁴═F).

Ketones of formula C4 wherein R² is hydrogen and R⁴ is methyl may be prepared from compound C6 (CAS# 123319-13-1; Tsukamoto et al., EP 0311313, publication date Dec. 4, 1989). Compound C6 may be oxidized by conventional methods as described above to give ketone C7. The acetyl protecting group of ketone C7 may be hydrolized by conventional methods such as with sodium methoxide in methanol or with refluxing aqueous HBr to give the parent amine which may be then converted to the Boc protected compound of formula C4 using conventional methods such as treatment with di-tert-butyl dicarbonate in DCM with triethylamine.

Ketones of formula C4 wherein R² is methyl and R⁴ is hydrogen may be prepared from compound C8 (CAS# 123319-30-2; Tsukamoto et al., EP 0311313, publication date Dec. 4, 1989). The ethyl carboxylate protecting group of ketone C8 may be removed by conventional methods such as with aqueous HBr in acetic acid or with aqueous HCl, usually at elevated temperatures, to give the parent amine which may then be converted to the Boc protected compound of formula C4 as described above.

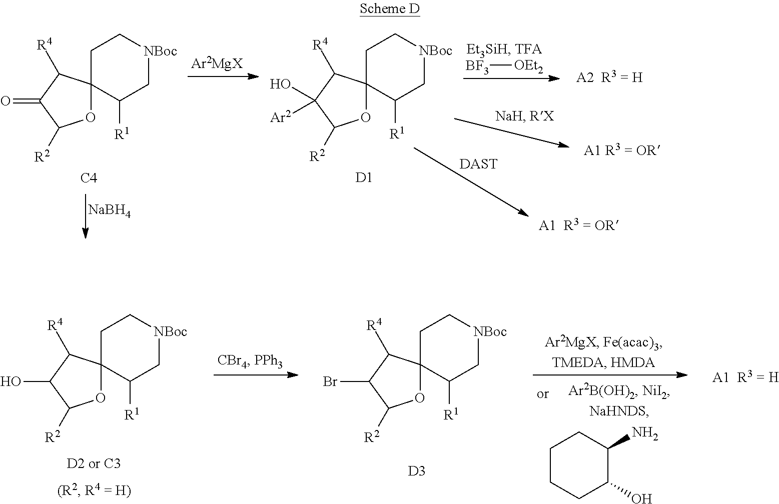

Compounds of formula A1 and A2 can be prepared according to Scheme D. Aryl Grignard reagents (Ar²MgX; X═Cl, Br, or I) can be purchased commercially or prepared from an aryl halide with reagents such as magnesium (for a review see Lai, Y. H. Synthesis 1981, 585-604) or isopropylmagnesium chloride (for a review see P. Knochel et al. Angew. Chem. Int. Ed. 2003, 42, 4302-4320; for the use of lithium chloride as an additive, see Krasovskiy and Knochel, Angew. Chem. Int. Ed. 2004, 43, 3333-3336). Addition of an aryl Grignard (Ar²MgX) to ketone compounds of formula C4 in a solvent such as THF at 0° C. to about room temperature gives alcohol compounds of formula D1. Alcohols of formula D1 can be treated with triethylsilane, trifluoroacetic acid, and boron trifluoride-diethyl etherate in a solvent such as dichloromethane at about −15° C. to about room temperature to give the reduced compounds of formula A2 (R³═H). Furthermore, compounds of formula D1 can also be alkylated with a base such as sodium hydride and an alkyl halide R′X (X═Br or I) in a solvent such as DMF or DMA to provide the corresponding compounds of formula A1 (R³═OR′). Additionally, compounds of formula D1 can also be treated with diethylaminosulfur trifluoride (DAST) in a solvent such as dichloromethane at −78° C. to about 0° C. to provide the corresponding compounds of formula A1 (R³═F). Compounds of formula C4 can also be reacted with a reducing agent such as sodium borohydride in methanol to give alcohols of formula D2. Compounds of formula D2 or C3 can be converted to bromides of formula D3 with triphenylphosphine and carbon tetrabromide in a solvent such as THF. Compounds of formula D3 can be coupled with aryl Grignard reagents (Ar²MgX; X═Cl, Br, I) in the presence of catalytic amounts of Fe(acac)₃, tetramethylethylenediamine (TMEDA) and hexamethylenetetramine (HMTA) in THF in a manner similar to that described by Cahiez et al., Angew. Chem. Int. Ed. 2007, 46 4364-4366, to give compounds of formula A1 (R³═H). Alternatively, compounds of formula D3 can be coupled with aryl boronic acids (Ar²B(OH)₂) in the presence of sodium hexamethyldisilazide (NaHMDS) and catalytic amounts of nickel iodide and trans-2-aminocyclohexanol in anhydrous isopropanol in a manner similar to that described by Gonzalez-Bobes and Fu, J. Am. Chem. Soc. 2006, 128, 5360-5361, to give compounds of formula A1 (R³═H).

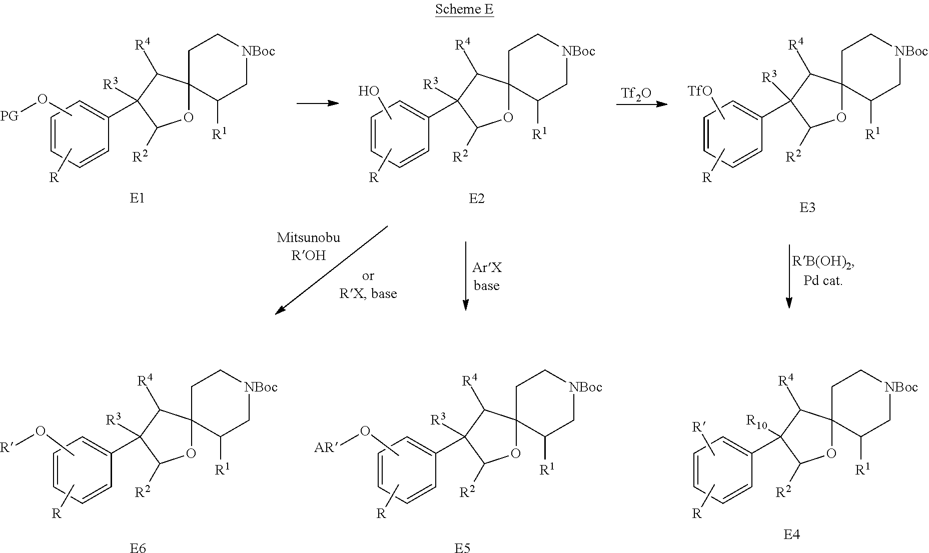

Compounds of formulae E4-E6 can be prepared according to Scheme E. Compounds of formula E1, where PG is benzyl (Bn), tert-butyldimethysilyl (TBS), triisopropylsilyl (TIPS) or tert-butyldiphenylsilyl (TBDPS), can be prepared as described in Scheme D for compounds of formula A1 and A2 (wherein compounds of formula A2 can converted to compounds of formula A1 by conventional means such as with di-tert-butyl dicarbonate in DCM with triethylamine). Compounds of formula E1 wherein PG is benzyl can be deprotected by conventional methods such as treatment with catalytic palladium on carbon under an atmosphere of hydrogen at about 10 to about 50 psi can give compounds of formula E2. Compounds of formula E1 wherein PG is TBS, TIPS, or TBDPS can be deprotected using conventional methods such as treatment with tetrabutylammonium fluoride in tetrahydrofuran to yield compounds of formula E2. Compounds of formula E2 can be treated with triflic anhydride in a solvent such as dichloromethane and the presence of a base such as pyridine to give compounds of formula E3. Triflates of formula E3 can be reacted with an aryl or alkyl boronic acid of formula (R′B(OH)₂) under palladium-catalyzed Suzuki cross-coupling conditions (for a review, see Chem. Rev. 1995, 95, 2457), to give the corresponding compounds of formula E4. For example, the coupling can be conducted using a catalytic amount of tetrakis(triphenylphosphine)-palladium(0) or (1,1′-bis-(diphenylphosphino)-ferrocene)palladium dichloride (Pd(dppf)Cl₂) in the presence of a base such as aqueous sodium carbonate, cesium carbonate, sodium hydroxide, or sodium ethoxide, in a solvent such as THF, dioxane, ethylene glycol dimethylether, DMF, ethanol or toluene. The temperature of the reaction may vary from about ambient temperature to about the reflux temperature of the solvent used. Further, compounds of formula E5 can be prepared by a nucleophilic aromatic substitution of a phenol of formula E2 with an electron deficient aryl halide (Ar′X; X═Cl or F) to form the biaryl ether of formula E5. This reaction is preferably run in the presence of a base such as potassium carbonate, sodium carbonate, cesium carbonate, NaHMDS, triethylamine or diisopropylethylamine. The solvent used may be DMF, DMA, NMP, DMSO, acetonitrile, tetrahydrofuran, dioxane or a combination of two or more of these solvents. Further, phenol compounds of formula E2 can be alkylated with an an alkyl halide (R′X; X═Cl, Br or I) using a base such as cesium carbonate, potassium carbonate, or sodium hydride in a solvent such as DMF, DMA, NMP, DMSO, dioxane, or acetonitrile, to yield compounds of formula E6. The temperature of the reaction may vary from about ambient temperature to about the reflux temperature of the solvent used and may be heated under conventional or microwave conditions. Sodium iodide or potassium iodide may be added to facilitate the alkylation. Alternatively, the phenol of compounds E2 can be reacted with alkyl alcohols (R′OH) under Mitsunobu reaction conditions (Organic Reactions 1992, 279, 22-27; Org. Prep. Proc. Int 1996, 28, 127-164; Eur. J. Org. Chem. 2004, 2763-2772) such as polystyrene-triphenylphosphine (PS-PPh₃) and di-tert-butyl azodicarboxylate (DBAD) to give compounds of formula E6.

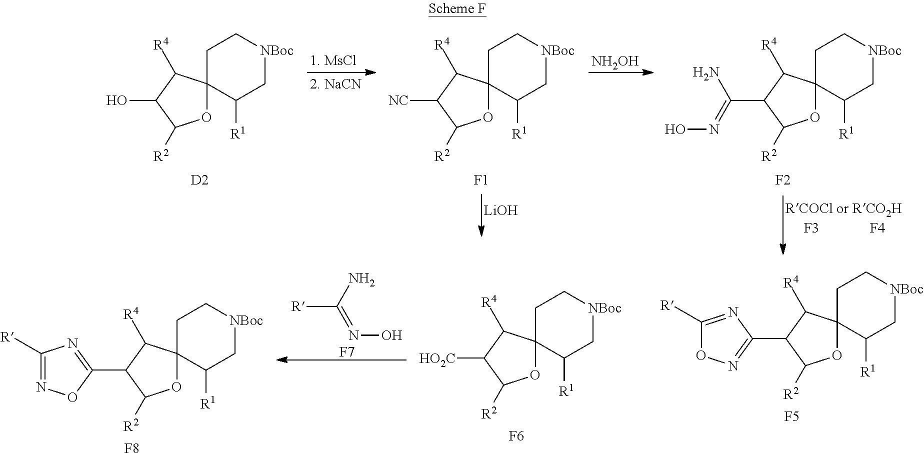

Compounds of formulae F5 and F8 can be prepared according to Scheme F. Alcohols of formula D2 can be treated with methanesulfonyl chloride in a solvent such as dichloromethane in the presence of a base such as triethylamine or DIEA. The meslyate intermediate can then be reacted with sodium cyanide in a suitable solvent such as DMF or DMSO at a temperature ranging from room temperature to about 90° C. to give nitrile compounds of formula F1. Nitriles of formula F1 can be treated with excess hydroxylamine hydrochloride and TEA in a solvent such as ethanol. The reaction is run at about 80° C. to reflux temperature of the solvent used to give hydroxyamidines of formula F2. Hydroxyamidines of formula F2 can be treated with acid chlorides of formula F3 in a solvent such as THF and the presence of a base such as DIEA or TEA. The reaction can be run at reflux of the solvent used and may be heated by conventional or microwave conditions to give oxadiazoles of formula F5. Alternatively, hydroxyamidines of formula F2 may be reacted with carboxylic acids of formula F4 in the presence of a coupling agent such as carbonyldiimidazole (CDI), O-(Benzotriazol-1-yl)-N,N,N′,N′-tetramethyluronium hexafluorophosphate (HBTU), and the like, in a solvent such as DMF in the presence of a base such as TEA or DIEA. The reaction may be run at RT followed by heating to about 110° C. to give oxadiazole compounds of formula F5. Nitriles of formula F1 can also be hydrolyzed by treatment with lithium hydroxide in a solvent such as ethanol/water at about reflux temperature to give carboxylic acids of formula F6. Carboxylic acids of formula F6 may then be converted to their acid chloride with thionyl chloride or oxalyl chloride and reacted with hydroxyamidines of formula F7 as described above to give oxadiazoles of formula F8. Alternatively, reactions of carboxylic acids of formula F6 with coupling agents such as CDI or HBTU and hydroxyamidines of formula F7 as described above to give oxadiazoles of formula F8.

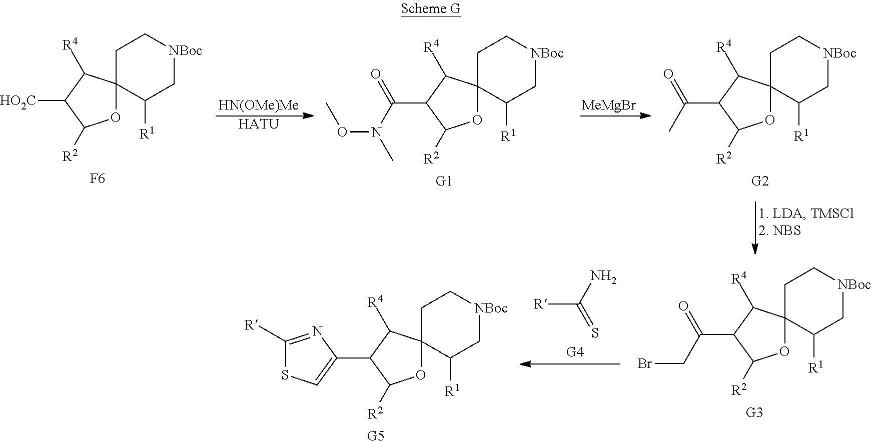

Thiazole compounds of formula G5 can be prepared according to Scheme G. Compounds of formula F6 can be treated with N,O-dimethylhydroxylamine hydrochloride in the presence a coupling agent such as O-(7-azabenzotriazol-1-yl)-N,N,N′,N′-tetramethyluronium hexafluorophosphate (HATU), and a base such as DIEA or TEA in a solvent such as dichloromethane to give the Weinreb amide of formula G1. The compound of formula G1 can be treated with methyl magnesium bromide in a solvent such as THF at about 0° C. to room temperature to give methyl ketone compounds of formula G2. Compounds of formula G2 can be treated with LDA in a solvent such as THF at about −78° C. followed by treatment with trimethylsilyl chloride (TMSCl). After isolation, the silyl enolate intermediate can be treated with sodium bicarbonate in THF followed by N-bromosuccinimide (NBS) at 0° C. to give α-bromoketone compounds of formula G3. Compounds of formula G3 can be reacted with thioamides of formula G4 in a solvent such as ethanol at a temperature ranging from about 80° C. to reflux temperature of the solvent used to give thiazole compounds of formula G5.

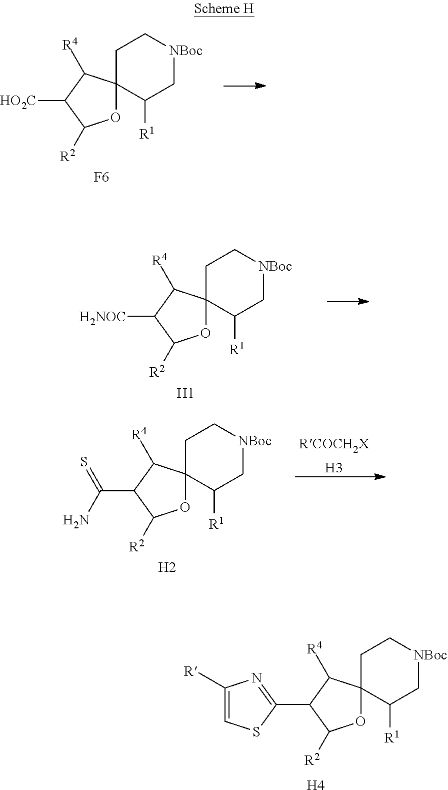

Thiazole compounds of formula H4 can be prepared according to Scheme H. Carboxylic acid compounds of formula F6 can be treated with ammonia in methanol in the presence a coupling agent such as HATU, and a base such as DIEA or TEA in a solvent such as dichloromethane to give the carboxamide of formula H1. Compounds of formula H1 can be treated with Lawesson's reagent in a solvent such as toluene. The reaction may be heated from about 65° C. to reflux temperature of the solvent used to provide thioamides of formula H2. Thioamides of formula H2 may be treated with α-haloketones of formula H3 (X═Cl or Br) in a solvent such as ethanol as described for Scheme G to give thiazole compounds of formula H4.

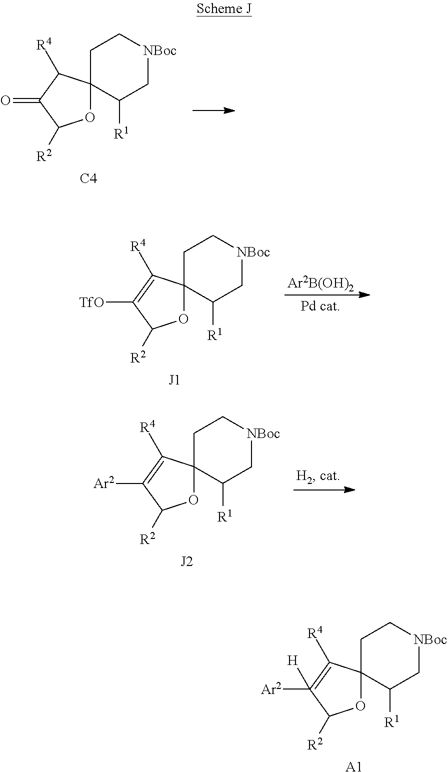

Scheme J illustrates another method for the preparation of compounds of formula A1. A ketone of formula C4 can be converted to a vinyl triflate of formula J1 by conventional methods such as treatment with a base such as LHMDS followed by a triflating agent such as 2-[N,N-bis(trifluoromethanesulphonyl)amino]-5-chloropyridine. Compounds of formula J1 can be reacted with an aryl boronic acid of formula (Ar²B(OH)₂) under palladium-catalyzed Suzuki cross-coupling conditions (as described in Scheme E; for a review, see Chem. Rev. 1995, 95, 2457) to give the corresponding compounds of formula J2. Olefin compounds of formula J2 can be reduced using conventional methods such as treatment with catalytic palladium on carbon under an atmosphere of hydrogen at atmospheric pressure to about 50 psi to give compounds of formula A1. Furthermore, the double bond of compounds of formula J2 may be reduced asymmetrically using catalytic asymmetric hydrogenation methods (for a review, see “Noyori Catalytic Asymmetric Hydrogenation.”, Lall, Manjinder S. Editors: Li, Jie Jack; Corey, E. J. Name Reactions for Functional Group Transformations (2007), p. 46-66. Publisher: John Wiley & Sons, Inc., Hoboken, N.J., and references therein) to give enantiomerically-enriched compounds of formula A1. For example, a compound of formula J2 wherein R¹, R², and R⁴ are hydrogen and Ar² is 3-benzyloxyphenyl can be hydrogenated at 200 psig and 70° C. in trifluoroethanol in the presence of a catalytic amount of (S)-1-[(R)-2-di-(4-fluorophenyl)phosphino)ferrocenyl]ethyldi-tert-butylphosphinerhodium(I)cyclooctadiene trifluoromethanesulfonate, [Rh(COD)((S)-TCFP)]⁻BF₄⁺, or [Rh(COD)((R)-TCFP)]⁻BF₄⁺ (see Hoge et al., J. Am. Chem. Soc. 2004, 126, 5966-5967) to give enantiomerically-enriched tert-butyl 3-[3-(benzyloxy)phenyl]-1-oxa-8-azaspiro[4.5]decane-8-carboxylate in 96-100% enantiomeric excess (ee).

### EXAMPLES

The following examples are intended to illustrate particular aspects of the compounds and methods described herein and are not intended to limit the scope of the claims. ¹H Nuclear magnetic resonance (NMR) spectra were obtained for the compounds in the following examples. Characteristic chemical shifts (δ) are given in parts-per-million (ppm) downfield from tetramethylsilane using conventional abbreviations for designation of major peaks, including s (singlet), d (doublet), t (triplet), q (quartet); m (multiplet), and br (broad). The following abbreviations are used for common solvents: CDCl₃ (deuterochloroform), DMSO-d₆ (deutero dimethylsulfoxide), and methanol-d₆ (deuteromethanol). Liquid chromatography-mass spectrometry (LCMS) were recorded using electrospray (ES) or atmospheric pressure chemical ionization (APCI) techniques.

### tert-butyl 4-allyl-4-hydroxypiperidine-1-carboxylate

tert-butyl 4-oxopiperidine-1-carboxylate (1000 g, 5.02 mol) was dissolved in allylbromide (1080 mL, 12.4 mol, 2.5 eq), THF (1000 mL) and saturated ammonium chloride solution (5000 mL). The reaction was cooled to 10° C. and zinc dust (650 g, 10 mol, 2 eq) was added portion wise. After addition the reaction mixture was stirred overnight. TLC was taken (heptane/EtOAc 7:1) and showed full conversion. The reaction mixture was diluted with water (5 L) and acidified with 10% H₂SO₄ to pH˜6. The reaction mixture was extracted with MTBE (3×). The organic layers were combined and extracted with saturated solution of NaHCO₃, brine and evaporated to give tert-butyl 4-allyl-4-hydroxypiperidine-1-carboxylate (1153 g, 95%).

### tert-butyl (3RS)-3-hydroxy-1-oxa-8-azaspiro[4.5]decane-8-carboxylate

tert-butyl 4-allyl-4-hydroxypiperidine-1-carboxylate (1153 g, 4.8 mol) was dissolved in tert-butanol (10 L) and water (4 L). To the solution sodium periodate (1124 g, 5.3 mol, 1.1 eq) was added and the mixture was stirred at 50° C. for 30 minutes. At 50° C. a solution of Na₂S₂O₅ (1007 g, 5.3 mol, 1.1 eq) in water (4.2 L) was added dropwise over 4 hours to the solution. After addition the reaction mixture was stirred for 7 hours at 50° C. and another 48 hours at RT. The mixture was transferred to an extraction vessel and the organic layer was separated from the aqueous layer. The aqueous layer was extracted (3×) with ethyl acetate. The organic layers were combined and washed with a saturated solution of Na₂S₂O₃ (3×) to give a colorless solution. The solution was washed with brine and evaporated to give crude product (987 g, 80%). The crude product was purified by flash chromatography (10-80% EtOAc/Heptane) to give the title compound (287 g, 35%). m/z 158 (MH⁺ minus Boc).

### tert-butyl (3RS)-3-bromo-1-oxa-8-azaspiro[4.5]decane-8-carboxylate

tert-butyl (3RS)-3-hydroxy-1-oxa-8-azaspiro[4.5]decane-8-carboxylate (60 g, 0.233 mol, 1 eq) was dissolved in dichloromethane and CBr₄ (92 g) was added. The mixture was stirred for 10 minutes and cooled to −5 C. Triphenylphosphine (73 g, 0.280 mol, 1.2 eq) was dissolved in DCM (250 mL) and added to the reaction mixture dropwise. The temperature was maintained between 0° C. and −5° C. during addition. After addition, the reaction mixture was allowed to warm up to room temperature and was stirred over night. TLC was taken (heptane/EtOAc 4:1) and showed full conversion. The dichloromethane was removed and MTBE was added. A white precipitate of triphenylphosphine was formed and filtered off. The crude product was purified by flash chromatography (4:1 heptane:EtOAc) to afford the title compound (31 g, 42%). Note: since CBr₄ and CHBr₃ can be present the column was first rinsed with heptane to remove CBr₄ and CHBr₃ from the crude product. m/z 264 (MH⁺ minus t-Bu).

### tert-butyl 3-oxo-1-oxa-8-azaspiro[4.5]decane-8-carboxylate

To a solution of tert-butyl (3RS)-3-hydroxy-1-oxa-8-azaspiro[4.5]decane-8-carboxylate (30 g, 120 mmol) in DMSO (300 mL) and triethylamine (58.5 mL, 420 mmol) at 0° C. was added pyridine sulfur trioxide (65 g, 410 mmol, CAS# 26412-87-3) in 10 g batches. The reaction was stirred at room temperature for 2 hr. The reaction was followed by TLC (10% IPA/Heptane) visualizing with iodine stain. The solution was poured into ice-cold water (1 L) and extracted with EA (3×300 mL). The combined organic layers were washed with 0.5 N HCl (3×400 mL), sodium sulfite (1×400 mL), and brine (1×400 mL). The organic layer was dried over magnesium sulfate, filtered and evaporated to give ˜33 g of an orange oil. The oil was purified by flash chromatography (2-20% IPA/Heptane) to give the title compound as a white solid (28.8 g, 97%). ¹H NMR (400 MHz, CDCl₃) δ ppm 1.44-1.52 (m, 9H), 1.61-1.73 (m, 2H), 1.75-1.87 (m, 2H), 2.38 (s, 2H), 3.40 (ddd, J=13.48, 10.24, 3.24 Hz, 2H), 3.67 (dt, J=13.57, 4.48 Hz, 2H), 4.04 (s, 2H).

### (3-{[5-(trifluoromethyl)pyridin-2-yl]oxy}phenyl)boronic acid

A mixture of 3-bromophenol (95 g, 0.552 moles), 2-chloro-5-(trifluoromethyl)pyridine (100 g, 1 eq) and K₂CO₃ (153 g, 2 eq) in DMF (1.2 L) was heated at 110° C. for 16 h under nitrogen atmosphere, cooled to room temperature and poured over crushed ice. The mixture was stirred for 1 h, filtered and dried to give a solid as brown granules (174 g, 99%). To a solution of this solid (150 g, 0.473 moles) and triisopropylborate (132 mL, 1.2 eq) in a THF:toluene mixture (1200 mL: 300 mL) was added n-BuLi (227 mL, 1.2 eq, 2.5 M in hexane) dropwise at −70° C. After addition, the mixture was stirred at the same temp for 2 h, slowly warmed to −20° C. and then quenched by dropwise addition of 1N HCl (250 mL). The mixture was stirred at 0° C. for 1 h, extracted with ethyl acetate, washed with water, brine, dried over Na₂SO₄ and concentrated to give a pink solid. The solid was purified by flash chromatography using 20% ethyl acetate in hexane to remove impurities followed by 40% ethyl acetate in hexane to give the title compound as light yellow powder (52 g, 39%). m/z 284 (MH⁺).

### tert-butyl (3RS)-3-(3-{[5-(trifluoromethyl)pyridin-2-yl]oxy}phenyl)-1-oxa-8-azaspiro[4.5]decane-8-carboxylate

In a vial, nickel iodide (58 mg, 0.19 mmol), trans 2-aminocyclohexanol (28 mg, 0.187 mmol), (3-{[5-(trifluoromethyl)pyridin-2-yl]oxy}phenyl)boronic acid (530 mg, 1.87 mmol), NaHMDS (362 mg, 1.87 mmol), and tert-butyl (3RS)-3-bromo-1-oxa-8-azaspiro[4.5]decane-8-carboxylate (300 mg, 0.937 mmol) were treated with anhydrous 2-propanol (2 mL, Aldrich) and immediately evacuated/filled with argon. The reaction mixture was capped and warmed to 60° C. for 6 hrs. The mixture was cooled and stood at room temperature overnight. The mixture was filtered through a plug of silica gel with EA and then evaporated to give an oil. The oil was purified by flash chromatography (0-40% EA then 0-10% IPA in Heptane) to give the title compound as an oil (307 mg, 69%). m/z 423 (MH⁺ minus t-Bu).

### (3RS)-3-(3-{[5-(trifluoromethyl)pyridin-2-yl]oxy}phenyl)-1-oxa-8-azaspiro[4.5]decane hydrochloride

A solution of tert-butyl (3RS)-3-(3-{[5-(trifluoromethyl)pyridin-2-yl]oxy}phenyl)-1-oxa-8-azaspiro[4.5]decane-8-carboxylate (307 mg, 0.64 mmol) in DCM (2 mL) was treated with 4 N HCl/dioxane (2 mL) and stirred for 1 hr. Solvent was evaporated overnight to give title compound as a light yellow solid (368 mg, quant.). m/z 379 (MH⁺).

### Example 1

### (3RS)—N-pyridazin-3-yl-3-(3-{[5-(trifluoromethyl)pyridin-2-yl]oxy}phenyl)-1-oxa-8-azaspiro[4.5]decane-8-carboxamide

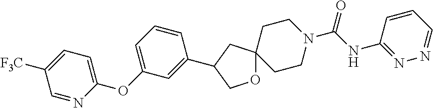

A mixture of (3RS)-3-(3-{[5-(trifluoromethyl)pyridin-2-yl]oxy}phenyl)-1-oxa-8-azaspiro[4.5]decane hydrochloride (283 mg, 0.682 mmol), phenyl pyridazin-3-ylcarbamate (147 mg, 0.682 mmol) in acetonitrile (5 mL) was treated with DIEA (0.475 mL, 2.73 mmol) and stirred in a vial at room temperature over 3 days. The solvent was evaporated and the residue was purified by reverse phase chromatography (10-95% acetonitrile/water/0.05% TFA). The desired fractions were evaporated. The residue was dissolved in acetonitrile and passed through a StratoSpheres™ PL-HCO₃ MP SPE tube (Polymer Laboratories, Amherst, Mass.) to neutralize any TFA. The eluent was evaporated to give an oil. The oil was dissolved in acetonitrile (˜5 mL) and diluted with an equal volume of water. This solution was lyophilized to give the title compound as a tan solid (230 mg, 68%). m/z 500 (MH⁺). ¹H NMR (400 MHz, DMSO-d₆) δ ppm 1.55-1.73 (m, 4H), 1.80 (dd, J=12.29, 10.58 Hz, 1H), 2.30 (dd, J=12.29, 7.85 Hz, 1H), 3.38-3.49 (m, 2H), 3.51-3.61 (m, 1H), 3.62-3.73 (m, 3H), 4.15 (t, J=7.68 Hz, 1H), 7.06 (dd, J=8.02, 2.22 Hz, 1H), 7.16 (s, 1H), 7.21 (s, 1H), 7.23 (s, 1 H), 7.39 (t, J=7.85 Hz, 1H), 7.55 (dd, J=9.05, 4.61 Hz, 1H), 7.98 (d, J=9.22 Hz, 1H), 8.23 (dd, J=8.70, 2.56 Hz, 1H), 8.57 (d, J=1.71 Hz, 1H), 8.82 (d, J=4.44 Hz, 1H), 9.83 (s, 1H).

### tert-butyl (3S)-3-(3-{[5-(trifluoromethyl)pyridin-2-yl]oxy}phenyl)-1-oxa-8-azaspiro[4.5]decane-8-carboxylate and tert-butyl (3R)-3-(3-{[5-(trifluoromethyl)pyridin-2-yl]oxy}phenyl)-1-oxa-8-azaspiro[4.5]decane-8-carboxylate

The racemic tert-butyl (3RS)-3-(3-{[5-(trifluoromethyl)pyridin-2-yl]oxy}phenyl)-1-oxa-8-azaspiro[4.5]decane-8-carboxylate (1.47 g) was separated by chiral SFC on a 30×250 mm ChiralPak OD-H column (20% MeOH/CO₂ @ 70 mL/min; 2 mL injections of a 100 mg/mL MeOH solution). The first eluting peak gave tert-butyl (3S)-3-(3-{[5-(trifluoromethyl)pyridin-2-yl]oxy}phenyl)-1-oxa-8-azaspiro[4.5]decane-8-carboxylate (559 mg, 75%; t_{R}=3.18 min (4.6×150 mm Chiralpak OD-H, 20% MeOH/CO₂ at 3 mL/min)) and the second eluting peak gave tert-butyl (3R)-3-(3-{[5-(trifluoromethyl)pyridin-2-yl]oxy}phenyl)-1-oxa-8-azaspiro[4.5]decane-8-carboxylate (670 mg, 89%; t_{R}=4.58 min (4.6×150 mm Chiralpak OD-H, 20% MeOH/CO₂ at 3 mL/min)). m/z 379 (MH⁺ minus Boc).

### (3S)-3-(3-{[5-(trifluoromethyl)pyridin-2-yl]oxy}phenyl)-1-oxa-8-azaspiro[4.5]decane hydrochloride

The title compound was prepared from tert-butyl (35)-3-(3-{[5-(trifluoromethyl)pyridin-2-yl]oxy}phenyl)-1-oxa-8-azaspiro[4.5]decane-8-carboxylate (559 mg) as described for (3RS)-3-(3-{[5-(trifluoromethyl)pyridin-2-yl]oxy}phenyl)-1-oxa-8-azaspiro[4.5]decane hydrochloride to give the title compound as a solid (485 mg, 94%). m/z 379 (MH⁺).

### Example 2

### (3S)—N-pyridazin-3-yl-3-(3-{[5-(trifluoromethyl)pyridin-2-yl]oxy}phenyl)-1-oxa-8-azaspiro[4.5]decane-8-carboxamide

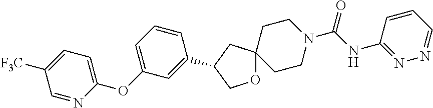

The title compound was prepared from (3S)-3-(3-{[5-(trifluoromethyl)pyridin-2-yl]oxy}phenyl)-1-oxa-8-azaspiro[4.5]decane hydrochloride (350 mg) as described for Example 1 to give the title compound as a solid (383 mg, 90%). m/z 500 (MH⁺). ¹H NMR (400 MHz, DMSO-d₆) δ ppm 1.54-1.72 (m, 4H), 1.80 (dd, J=12.46, 10.41 Hz, 1H), 2.30 (dd, J=12.29, 7.85 Hz, 1H), 3.37-3.48 (m, 2H), 3.50-3.61 (m, 1H), 3.61-3.72 (m, 3H), 4.14 (t, J=7.68 Hz, 1H), 7.05 (dd, J=8.02, 2.22 Hz, 1H), 7.16 (s, 1H), 7.22 (d, J=8.53 Hz, 2H), 7.39 (t, J=7.85 Hz, 1H), 7.55 (dd, J=9.05, 4.61 Hz, 1H), 7.98 (dd, J=9.05, 1.19 Hz, 1H), 8.23 (dd, J=8.70, 2.56 Hz, 1H), 8.53-8.59 (m, 1H), 8.82 (dd, J=4.61, 1.19 Hz, 1H), 9.84 (s, 1H).

### (3R)-3-(3-{[5-(trifluoromethyl)pyridin-2-yl]oxy}phenyl)-1-oxa-8-azaspiro[4.5]decane hydrochloride

The title compound was prepared from tert-butyl (3R)-3-(3-{[5-(trifluoromethyl)pyridin-2-yl]oxy}phenyl)-1-oxa-8-azaspiro[4.5]decane-8-carboxylate (670 mg) as described for (3RS)-3-(3-{[5-(trifluoromethyl)pyridin-2-yl]oxy}phenyl)-1-oxa-8-azaspiro[4.5]decane hydrochloride to give the title compound as a solid (608 mg, quant.). m/z 379 (MH⁺).

### Example 3

### (3R)—N-pyridazin-3-yl-3-(3-{[5-(trifluoromethyl)pyridin-2-yl]oxy}phenyl)-1-oxa-8-azaspiro[4.5]decane-8-carboxamide

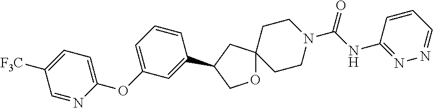

The title compound was prepared from (3R)-3-(3-{[5-(trifluoromethyl)pyridin-2-yl]oxy}phenyl)-1-oxa-8-azaspiro[4.5]decane hydrochloride (350 mg) as described for Example 1 to give the title compound as a solid (279 mg, 66%). m/z 500 (MH⁺). ¹H NMR (400 MHz, DMSO-d₆) δ ppm 1.54-1.73 (m, 4H), 1.79 (dd, J=12.29, 10.58 Hz, 1H), 2.30 (dd, J=12.29, 7.85 Hz, 1H), 3.37-3.50 (m, 2H), 3.51-3.61 (m, 1H), 3.61-3.74 (m, 3H), 4.14 (t, J=7.68 Hz, 1H), 7.02-7.09 (m, 1H), 7.16 (s, 1H), 7.22 (d, J=8.53 Hz, 2H), 7.39 (t, J=7.85 Hz, 1H), 7.55 (dd, J=9.05, 4.61 Hz, 1H), 7.98 (dd, J=8.88, 1.37 Hz, 1H), 8.23 (dd, J=8.70, 2.56 Hz, 1 H), 8.57 (d, J=1.71 Hz, 1H), 8.82 (dd, J=4.44, 1.37 Hz, 1H), 9.84 (s, 1H).

### tert-butyl (3RS)-3-[3-(benzyloxy)phenyl]-1-oxa-8-azaspiro[4.5]decane-8-carboxylate

A solution of the 3-benzyloxyphenylmagnesium bromide (85 mL, 1.0 M in THF; Aldrich) was added dropwise via a syringe pump over about 4 hr to a stirred solution of tert-butyl (3RS)-3-bromo-1-oxa-8-azaspiro[4.5]decane-8-carboxylate (15 g, 47 mmol), Fe(acac)₃ (836 mg, 2.34 mmol), TMEDA (0.711 mL, 4.68 mmol), and HMTA (332 mg, 2.34 mmol) in THF (300 mL) at 0 C. Upon completion of the reaction as judged by LC/MS it was quenched with brine (300 mL) and extracted with ethyl acetate (200 mL). The resulting mixture was filtered and the layers were separated. The organic was dried over magnesium sulfate, filtered, and concentrated to give an oil. The oil was treated with methanol (˜300 mL) and a solid precipitated upon standing. The mixture was filtered and the filter cake was washed with methanol (2×50 mL). The filtrate was evaporated to give the crude oil. The oil was purified by flash chromatography (5-40% EA/Heptane) to give the title compound as an oil (14.11 g, 71%). m/z 368 (MH⁺ minus t-Bu).

### tert-butyl (3RS)-3-(3-hydroxyphenyl)-1-oxa-8-azaspiro[4.5]decane-8-carboxylate, tert-butyl (3R)-3-(3-hydroxyphenyl)-1-oxa-8-azaspiro[4.5]decane-8-carboxylate and tert-butyl (3S)-3-(3-hydroxyphenyl)-1-oxa-8-azaspiro[4.5]decane-8-carboxylate

To a solution of tert-butyl (3RS)-3-[3-(benzyloxy)phenyl]-1-oxa-8-azaspiro[4.5]decane-8-carboxylate (14.11 g, 33.31 mmol) in 50% EA/methanol (100 mL) was added 10% Pd/C (200 mg, CAS#7440-05-3) catalyst under nitrogen, and the mixture was stirred under 40 psi of hydrogen overnight. The mixture was filtered through a plug of celite and the resulting filtrate evaporated to give racemic tert-butyl (3RS)-3-(3-hydroxyphenyl)-1-oxa-8-azaspiro[4.5]decane-8-carboxylate as a white solid (10.74 g, 96.7%). The racemate was separated by chiral SFC on a 50×250 mm ChiralPak AD-H column (20% 50:50 MeOH:EtOH/CO₂ @ 200 mL/min; 4 mL injections of a 50 mg/mL EtOH solution). The first eluting peak gave tert-butyl (3R)-3-(3-hydroxyphenyl)-1-oxa-8-azaspiro[4.5]decane-8-carboxylate (5.3 g, 99%; t_{R}=1.25 min (4.6×100 mm Chiralpak AD-H, 20% EtOH/CO₂ at 3 mL/min)) and the second eluting peak gave tert-butyl (3S)-3-(3-hydroxyphenyl)-1-oxa-8-azaspiro[4.5]decane-8-carboxylate (5.2 g, 97%; t_{R}=1.64 min (4.6×100 mm Chiralpak AD-H, 20% EtOH/CO₂ at 3 mL/min)). m/z 278 (MH⁺ minus t-Bu).

### Phenyl pyridazin-3-ylcarbamate

To a solution of 3-amino-6-chloropyridazine (19.2 g, 148 mmol; CAS# 5469-69-2) in EtOH (500 mL) was added 10% Pd catalyst on 1940 carbon (unreduced, 55% water). Triethylamine (50 mL) was added and the mixture was hydrogenated under 500 psi/mole for 1.9 h. The reaction was filtered and the ethanol was washed with aqueous NH₄Cl. The organic layer was concentrated to give pyridazin-3-amine as a white solid (11 g, 78% yield). MS (APCI 10V) AP+1 96.2. To a suspension of pyridazin-3-amine (5 g, 50 mmol) in THF (50 mL) and CH₃CN (70 mL) was added pyridine (5.10 mL, 63.1 mmol) followed by phenyl chloroformate (6.95 mL, 55.2 mmol) slowly. The reaction was stirred overnight. The reaction was filtered to remove the precipitate. The filtrate was concentrated and then taken up in CH₂Cl₂ which was then washed with water. The organic layer was dried using SPE phase separators and concentrated. The residue was purified by silica gel column chromatography (0-5% MeOH/CH₂Cl₂). An undesired side product eluted first followed by the title compound which was concentrated to give a white solid (7.5 g, 70% yield). MS (APCI 10V) AP+1 216.12; ¹H NMR (400 MHz, DMSO-d₆) δ ppm 7.20-7.24 (m, 2H) 7.25-7.28 (m, 1H) 7.39-7.44 (m, 2H) 7.64-7.69 (m, 1H) 8.05 (dd, 1H) 8.94 (dd, 1H) 11.34 (s, 1H).

### Phenyl pyridin-3-ylcarbamate

To a stirred solution of 3-aminopyridine (51.7 g, 0.549 moles) in THF (900 mL) at −10° C. was added pyridine (52.1 g, 0.659 moles) in a stream over a 10 min period, followed by the dropwise addition of phenyl chloroformate (90 g, 0.575 moles) over a 20 min period. The reaction temperature increased to 5° C. A precipitate formed during the addition. The resulting suspension was stirred at temperatures reaching ambient temperature over the next 3 h. The reaction mixture was partitioned between water (2 L) and EtOAc (1.5 L). The aqueous portion was extracted with EtOAc (1 L). The combined organic portions were dried (MgSO₄) and concentrated in vacuo to a damp solid residue. This was suspended in EtOAc:ether (1:1, 600 mL). The resulting suspension was stirred at −10° C. for 2 h and filtered. The solid was rinsed with EtOAc:ether (1:1, 100 mL) and pressed dry under suction. Further drying in vacuo at 35° C. for 7 h provided 104 g (88%) of product. Analysis Calcd for C₁₂H₁₀N₂O₂: C, 67.28; H, 4.71; N, 13.08. Found: C, 67.15; H, 4.76; N, 12.87.

### Examples 4-6

Step 1. Separate vials of 5-bromo-2-chloropyridine (0.5 mmol) or 5-chloro-2-chloropyridine (0.5 mmol) in dioxane (2 mL) were treated with tert-butyl (3RS)-3-(3-hydroxyphenyl)-1-oxa-8-azaspiro[4.5]decane-8-carboxylate (0.25 mmol), DMA (0.25 mL), and NaHMDS (0.6 N in toluene; 0.5 mL, 0.300 mmol). The mixtures were heated under microwave irradiation at 185° C. for 1 h in a Biotage Initiator 60. Upon completion of the reaction, the solvents were evaporated in vacuo. The residues were reconstituted in dichloroethane (2 mL) and washed with water (2×1 mL). The organic layers were passed through Celite. The filtrates were concentrated. Step 2. The resulting residues were dissolved in 20% trifluoroacetic acid/dichloromethane and shaken at room temp for 2 h. The volatiles were removed in vacuo to provide the crude amines as a TFA salts. Step 3. The residues were dissolved in DMSO (1 mL). 0.5 mL of these solutions (0.125 mmol) were combined with a 0.5 M solution of phenyl pyridazin-3-ylcarbamate or phenyl pyridin-3-ylcarbamate in DMSO (0.25 mL, 0.125 mmol) and N-methyl morpholine (0.100 mL). The reactions were shaken at 60° C. for 2 h. The reaction mixtures were purified by reverse phase HPLC (acetonitrile/water/0.05% TFA) to give racemic Examples 4-6. The purified compounds were analyzed by LCMS (Phenomenex Gemini C18 4.6×50 mm 5 μm; 10 mM ammonium bicarbonate pH 8.2/MeCN).

| | | | m/z |
|---|---|---|---|
| Ex. | Name | t_{R} (min) | (MH⁺) |
| 4 | (3RS)-3-{3-[(5-chloropyridin-2-yl)oxy]phenyl}- | 1.64 | 466.35 |
| | N-pyridazin-3-yl-1-oxa-8-azaspiro[4.5]decane- | | |
| | 8-carboxamide | | |
| 5 | (3RS)-3-{3-[(5-bromopyridin-2-yl)oxy]phenyl}- | 1.69 | 510.35 |
| | N-pyridazin-3-yl-1-oxa-8-azaspiro[4.5]decane- | | |
| | 8-carboxamide | | |
| 6 | (3RS)-3-{3-[(5-bromopyridin-2-yl)oxy]phenyl}- | 1.72 | 509.35 |
| | N-pyridin-3-yl-1-oxa-8-azaspiro[4.5]decane- | | |
| | 8-carboxamide | | |

### tert-butyl (3R)-3-{3-[(5-bromopyridin-2-yl)oxy]phenyl}-1-oxa-8-azaspiro[4.5]decane-8-carboxylate

To a mixture of tert-butyl (3R)-3-(3-hydroxyphenyl)-1-oxa-8-azaspiro[4.5]decane-8-carboxylate (2.52 g, 7.56 mmol) and cesium carbonate (5.0 g, 15.3 mmol) in DMF (25 mL) was added 5-bromo-2-fluoropyridine (1.09 mL, 10.6 mmol, CAS# 766-11-0) and stirred at RT overnight. More 5-bromo-2-fluoropyridine (0.6 mL, 5.83 mmol) was added and the reaction was stirred overnight at RT. The reaction was treated with water (50 mL) and ethyl acetate (150 mL) and the layers were separated. Washed the organic with satd ammonium chloride (3×50 mL), dried over magnesium sulfate, filtered, and concentrated to give ˜5 g of the crude oil. The oil was diluted with DCM and purified by flash chromatography (5-30% EA/Heptane) to give the title compound as a gum (3.73 g, 97%). m/z 433 (MH⁺ minus tBu). ¹H NMR (400 MHz, CDCl₃) δ ppm 1.47 (s, 9H), 1.53-1.63 (m, 1H), 1.65-1.77 (m, 3H), 1.83 (dd, J=12.29, 10.24 Hz, 1H), 2.27 (dd, J=12.63, 8.19 Hz, 1H), 3.31-3.42 (m, 2H), 3.48-3.58 (m, 1H), 3.64 (br. s., 2H), 3.81 (t, J=8.88 Hz, 1H), 4.23 (t, J=8.02 Hz, 1H), 6.85 (d, J=8.53 Hz, 1H), 6.96-7.03 (m, 2H), 7.10 (d, J=7.85 Hz, 1H), 7.35 (t, J=7.85 Hz, 1H), 7.78 (dd, J=8.70, 2.56 Hz, 1H), 8.22 (d, J=2.73 Hz, 1H).

### (3R)-3-{3-[(5-bromopyridin-2-yl)oxy]phenyl}-1-oxa-8-azaspiro[4.5]decane hydrochloride

To tert-butyl (3R)-3-{3-[(5-bromopyridin-2-yl)oxy]phenyl}-1-oxa-8-azaspiro[4.5]decane-8-carboxylate (3.73 g, 7.62 mmol) in DCM (50 mL) was added 4 N HCl/dioxane (20 mL, 80 mmol) at 0° C. The solution was stirred for 1.5 hr at 0° C. and then warmed to RT and stirred another 1.5 hr. The mixture was evaporated to give the title compound as a foam (3.84 g, quantitative). m/z 389 (MH⁺). ¹H NMR (400 MHz, DMSO-d₆) δ ppm 1.76-1.91 (m, 5H), 2.30 (dd, J=12.29, 7.85 Hz, 1H), 2.96-3.14 (m, 4H), 3.51-3.55 (m, 1H), 3.61-3.69 (m, 1 H), 4.13 (t, J=7.68 Hz, 1H), 6.99 (dd, J=7.85, 2.39 Hz, 1H), 7.02 (d, J=8.88 Hz, 1H), 7.09 (s, 1H), 7.16 (d, J=7.51 Hz, 1H), 7.35 (t, J=7.85 Hz, 1H), 8.05 (dd, J=8.70, 2.56 Hz, 1H), 8.26 (d, J=2.73 Hz, 1H), 8.86 (br. s., 2H).

### (3S)-3-{3-[(5-bromopyridin-2-yl)oxy]phenyl}-1-oxa-8-azaspiro[4.5]decane hydrochloride

To a mixture of tert-butyl (3S)-3-(3-hydroxyphenyl)-1-oxa-8-azaspiro[4.5]decane-8-carboxylate (700 mg, 2.10 mmol) and cesium carbonate (1.37 g, 4.20 mmol) in DMF (7 mL) was added 5-bromo-2-chloropyridine (566 mg, 2.94 mmol, CAS# 53939-30-3) and stirred at 90° C. overnight. More 5-bromo-2-chloropyridine (100 mg, 0.52 mmol) and cesium carbonate (300 mg, 0.921 mmol) were added and the reaction was stirred overnight at 90° C. The reaction was treated with water and ethyl acetate and the layers were separated. The organic was washed with satd ammonium chloride, dried over magnesium sulfate, filtered, and concentrated to give the crude product. The crude product was dissolved with DCM (10 mL) and treated with 4 N HCl/dioxane (2 mL, 8 mmol). The mixture was stirred for 4 hr at RT and then evaporated to give the title compound as an oil (894 mg, quantitative). m/z 389 (MH⁺).

### Example 7

### (3R)-3-{3-[(5-bromopyridin-2-yl)oxy]phenyl}-N-pyridin-3-yl-1-oxa-8-azaspiro[4.5]decane-8-carboxamide

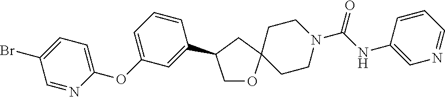

A mixture of (3R)-3-{3-[(5-bromopyridin-2-yl)oxy]phenyl}-1-oxa-8-azaspiro[4.5]decane hydrochloride (447 mg, 1.05 mmol) and phenyl pyridin-3-ylcarbamate (225 mg, 1.05 mmol) in acetonitrile (5 mL) was treated with DIEA (0.732 mL, 4.20 mmol) and stirred at RT overnight. The solution was concentrated and the residue was purified by reverse phase chromatography (5-95% acetonitrile/water/0.05% TFA). The desired fractions were concentrated and the residue was partitioned between ethyl acetate and satd sodium bicarbonate solution. The organic phase was dried over sodium sulfate, filtered, and concentrated. The residue was purified by flash chromatography (2-40% IPA/EA) to give an oil. The oil was dried on the high vacuum to give a foam. The foam was further dried overnight on the high vacuum at 30° C. to give the title compound as a foam (419 mg, 76%). m/z 509 (MH⁺). ¹H NMR (400 MHz, CDCl₃) δ ppm 1.63-1.75 (m, 1H), 1.75-1.92 (m, 4H), 2.30 (dd, J=12.63, 8.19 Hz, 1H), 3.40-3.62 (m, 3H), 3.76-3.88 (m, 3H), 4.21-4.30 (m, 1H), 6.85 (d, J=8.53 Hz, 1H), 6.95-7.04 (m, 3H), 7.10 (d, J=7.85 Hz, 1H), 7.30 (dd, J=8.19, 4.78 Hz, 1H), 7.36 (t, J=7.85 Hz, 1 H), 7.79 (dd, J=8.70, 2.56 Hz, 1H), 8.17 (d, J=8.53 Hz, 1H), 8.20-8.27 (m, 1H), 8.60 (d, J=2.39 Hz, 1H).

### Example 8

### (3S)-3-{3-[5-bromopyridin-2-yl)oxy]phenyl}-N-pyridin-3-yl-1-oxa-8-azaspiro[4.5]decane-8-carboxamide

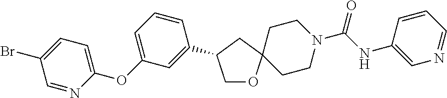

A mixture of (3S)-3-{3-[(5-bromopyridin-2-yl)oxy]phenyl}-1-oxa-8-azaspiro[4.5]decane hydrochloride (447 mg, 1.05 mmol) and phenyl pyridin-3-ylcarbamate (225 mg, 1.05 mmol) in acetonitrile (5 mL) was treated with DIEA (0.732 mL, 4.20 mmol) and stirred at RT overnight. The solution was concentrated and the residue was purified by reverse phase chromatography (5-95% acetonitrile/water/0.05% TFA). The desired fractions were concentrated and the residue was partitioned between ethyl acetate and satd sodium bicarbonate solution. The organic phase was dried over sodium sulfate, filtered, and concentrated. The oil was dissolved in ethanol and evaporated to give a foam under high vacuum. The foam was further dried overnight on the high vacuum at 30° C. to give the title compound as a foam (419 mg, 76%). m/z 509 (MH⁺). ¹H NMR (400 MHz, CDCl₃) δ ppm 1.64-1.74 (m, 1H), 1.78-1.92 (m, 4H), 2.30 (dd, J=12.63, 8.19 Hz, 1H), 3.41-3.62 (m, 3H), 3.76-3.86 (m, 3H), 4.25 (t, J=8.02 Hz, 1H), 6.85 (d, J=8.53 Hz, 1H), 6.95-7.03 (m, 3H), 7.10 (d, J=7.85 Hz, 1H), 7.30 (dd, J=8.36, 4.95 Hz, 1H), 7.36 (t, J=7.85 Hz, 1H), 7.79 (dd, J=8.70, 2.56 Hz, 1H), 8.16 (d, J=8.19 Hz, 1H), 8.19-8.28 (m, 2H), 8.59 (d, J=2.39 Hz, 1H).

### Example 9

### (3R)-3-{3-[(5-bromopyridin-2-yl)oxy]phenyl}-N-pyridazin-3-yl-1-oxa-8-azaspiro[4.5]decane-8-carboxamide

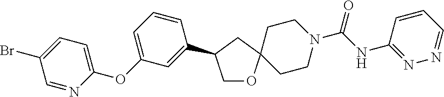

A mixture of (3R)-3-{3-[(5-bromopyridin-2-yl)oxy]phenyl}-1-oxa-8-azaspiro[4.5]decane hydrochloride (3.14 g, 7.37 mmol) phenyl pyridazin-3-ylcarbamate (1.90 g, 8.84 mmol) in acetonitrile (50 mL) was treated with DIEA (5.13 mL, 29.5 mmol) and stirred at RT overnight. The solution was concentrated. The residue was dissolved in EA (150 mL) and treated with water (50 mL) and brine (50 mL). The layers were separated and the organic was washed with brine (50 mL). The organic was dried over magnesium sulfate, filtered, and evaporated to give an oil. The oil was purified by flash chromatography (50-90% EA/Heptane with 5% IPA in the EA solvent) to give 3.8 g of a foam. The foam was dissolved mostly in EtOH (50 mL) and transferred to a pear shaped flask. This was seeded with a seed crystal and allowed to stand until crystals started to form. The remaining material was then transferred to this flask with EtOH (50 mL) and this was then allowed to stand for 1 hour. The mixture was then placed in the freezer for about 1.5 hr. The solid was collected and washed with a small amount of cold ethanol. This solid was dried on the high vac over the weekend. After drying, the ppt. gave the title compound as a white solid (2.96 g, 79%). m/z 510 (MH⁺). ¹H NMR (400 MHz, DMSO-d₆) δ ppm 1.55-1.73 (m, 4H), 1.79 (dd, J=12.46, 10.41 Hz, 1H), 2.29 (dd, J=12.29, 7.85 Hz, 1H), 3.38-3.49 (m, 2H), 3.50-3.60 (m, 1H), 3.62-3.73 (m, 3H), 4.14 (t, J=7.85 Hz, 1H), 6.99 (dd, J=7.85, 1.71 Hz, 1H), 7.03 (d, J=8.88 Hz, 1H), 7.10 (s, 1H), 7.17 (d, J=7.85 Hz, 1H), 7.36 (t, J=7.85 Hz, 1H), 7.55 (dd, J=9.05, 4.61 Hz, 1H), 7.98 (dd, J=8.88, 1.37 Hz, 1H), 8.05 (dd, J=8.88, 2.73 Hz, 1H), 8.28 (d, J=2.39 Hz, 1 H), 8.82 (dd, J=4.44, 1.37 Hz, 1H), 9.84 (s, 1H). Assignment of absolute stereochemistry was confirmed by x-ray crystallography of crystals of the title compound obtained from ethanol.

### Example 10

### (3S)-3-{3-[(5-bromopyridin-2-yl)oxy]phenyl}-N-pyridazin-3-yl-1-oxa-8-azaspiro[4.5]decane-8-carboxamide

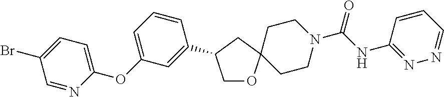

A mixture of (3S)-3-{3-[(5-bromopyridin-2-yl)oxy]phenyl}-1-oxa-8-azaspiro[4.5]decane hydrochloride (447 g, 1.05 mmol) and phenyl pyridazin-3-ylcarbamate (271 mg, 1.26 mmol) in acetonitrile (5 mL) was treated with DIEA (0.732 mL, 4.20 mmol) and stirred at RT for 3 hrs. The solution was concentrated. The residue was purified by reverse phase chromatography (5-95% acetonitrile/water/0.05% TFA), concentrated the desired fractions, and partitioned between ethyl acetate and satd sodium bicarbonate solution. The organic phase was dried over sodium sulfate, filtered, and concentrated. The residue was dissolved in ethanol and evaporated to give a foam under high vacuum. The foam was dried overnight on the high vacuum at 30° C. to give the title compound as a foam (456 mg, 84%). m/z 510 (MH⁺). ¹H NMR (400 MHz, CDCl₃) δ ppm 1.63-1.74 (m, 1H), 1.74-1.93 (m, 5H), 2.30 (dd, J=12.63, 8.19 Hz, 1H), 3.40-3.62 (m, 3H), 3.79-3.96 (m, 3 H), 4.26 (t, J=8.19 Hz, 1H), 6.86 (d, J=8.88 Hz, 1H), 6.97-7.04 (m, 2H), 7.11 (d, J=7.85 Hz, 1H), 7.36 (t, J=7.68 Hz, 1H), 7.46 (dd, J=9.22, 4.78 Hz, 1H), 7.72-7.83 (m, 1H), 8.22 (d, J=2.39 Hz, 1H), 8.39 (br. s., 1 H), 8.78 (br. s., 1H).

### tert-butyl (3R)-3-{3-[(5-chloropyridin-2-yl)oxy]phenyl}-1-oxa-8-azaspiro[4.5]decane-8-carboxylate

To a mixture of tert-butyl (3R)-3-(3-hydroxyphenyl)-1-oxa-8-azaspiro[4.5]decane-8-carboxylate (2.82 g, 8.47 mmol) and cesium carbonate (5.52 g, 16.9 mmol) in DMF (5 mL) was added 5-chloro-2-fluoropyridine (1.5 mL, 15 mmol) and stirred at RT over several nights. Water (50 mL) and ethyl acetate (150 mL) were added and the layers were separated. The organic was washed with saturated ammonium chloride (3×50 mL), dried over magnesium sulfate, filtered, and concentrated to give ˜5 g of the crude oil. The oil was purified by flash chromatography (5-30% EA/Heptane) to give the title compound as a gum (3.80 g, 96.4%). m/z 389 (MH⁺ minus t-Bu). ¹H NMR (400 MHz, CDCl₃) δ ppm 1.47 (s, 9H), 1.53-1.59 (m, 1H), 1.65-1.77 (m, 3H), 1.82 (dd, J=12.63, 10.24 Hz, 1H), 2.27 (dd, J=12.63, 8.19 Hz, 1H), 3.30-3.41 (m, 2H), 3.48-3.58 (m, 1H), 3.62 (br. s., 2H), 3.80 (t, J=8.88 Hz, 1H), 4.23 (t, J=8.02 Hz, 1H), 6.89 (d, J=8.88 Hz, 1H), 6.96-7.02 (m, 2H), 7.10 (d, J=7.85 Hz, 1H), 7.35 (t, J=7.68 Hz, 1H), 7.66 (dd, J=8.53, 2.73 Hz, 1H), 8.13 (d, J=2.73 Hz, 1H).

### (3R)-3-{3-[(5-chloropyridin-2-yl)oxy]phenyl}-1-oxa-8-azaspiro[4.5]decane hydrochloride

To tert-butyl (3R)-3-{3-[(5-chloropyridin-2-yl)oxy]phenyl}-1-oxa-8-azaspiro[4.5]decane-8-carboxylate (3.77 g, 8.47 mmol) in DCM (50 mL) was added 4 N HCl/dioxane (20 mL, 80 mmol) at 0 C. The solution was stirred for 1.5 hr at 0° C. and then warmed to RT and stirred another 1.5 hr. The mixture was evaporated to give the title compound as a foam (3.92 g, quant.). m/z 345 (MH⁺). ¹H NMR (400 MHz, DMSO-d₆) δ ppm 1.76-1.91 (m, 5H), 2.30 (dd, J=12.29, 7.85 Hz, 1H), 2.97-3.15 (m, 4H), 3.51-3.59 (m, 1H), 3.62-3.68 (m, 1H), 4.14 (t, J=7.68 Hz, 1H), 6.99 (dd, J=8.19, 2.39 Hz, 1H), 7.05-7.11 (m, 2H), 7.16 (d, J=8.19 Hz, 1H), 7.36 (t, J=7.85 Hz, 1H), 7.96 (dd, J=8.53, 2.73 Hz, 1H), 8.20 (d, J=2.73 Hz, 1H), 8.94 (br. s., 2H).

### Example 11

### (3R)-3-{3-[(5-chloropyridin-2-yl)oxy]phenyl}-N-pyridazin-3-yl-1-oxa-8-azaspiro[4.5]decane-8-carboxamide

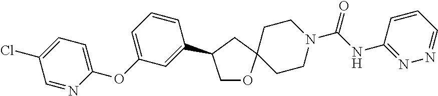

A mixture of (3R)-3-{3-[(5-chloropyridin-2-yl)oxy]phenyl}-1-oxa-8-azaspiro[4.5]decane hydrochloride (3.23 g, 8.47 mmol) and phenyl pyridazin-3-ylcarbamate (2.19 g, 10.2 mmol) in acetonitrile (60 mL) was treated with DIEA (6.0 mL, 34 mmol) and stirred at RT overnight. The reaction was concentrated. The residue was dissolved in EA (150 mL) and treated with water (25 mL) and brine (25 mL). The layers were separated and the organic was washed with brine (50 mL). The organic was dried over magnesium sulfate, filtered and evaporated to give an oil. The oil was dissolved in a small amount of DCM and purified by flash chromatography (60-100% EA/Heptane with 5% IPA premixed in the EA solvent). The resulting foam was dissolved mostly in EtOH (50 mL) and transferred to a pear shaped flask. After a short period of time (˜5 min) crystals started to form. The remaining material was transferred to this flask with EtOH (50 mL) and this was then allowed to stand for 1 hr. The mixture was then placed in the freezer for 2 hr. The solid was collected and washed with a small amount of cold ethanol. This solid was dried on the high vac overnight to give the title compound as a white solid (2.64 g, 67%). m/z 466 (MH⁺). ¹H NMR (400 MHz, DMSO-d₆) δ ppm 1.54-1.73 (m, 4H), 1.79 (dd, J=12.29, 10.58 Hz, 1H), 2.29 (dd, J=12.63, 7.85 Hz, 1H), 3.37-3.49 (m, 2H), 3.49-3.61 (m, 1H), 3.61-3.73 (m, 3H), 4.14 (t, J=7.68 Hz, 1H), 6.99 (dd, J=8.02, 2.22 Hz, 1H), 7.08 (d, J=8.88 Hz, 2H), 7.17 (d, J=7.85 Hz, 1H), 7.36 (t, J=7.85 Hz, 1H), 7.55 (dd, J=9.05, 4.61 Hz, 1H), 7.90-8.02 (m, 2H), 8.21 (d, J=2.73 Hz, 1H), 8.82 (d, J=4.78 Hz, 1H), 9.84 (s, 1H).

### Example 12

### (3S)-3-{3-[(5-chloropyridin-2-yl)oxy]phenyl}-N-pyridazin-3-yl-1-oxa-8-azaspiro[4.5]decane-8-carboxamide

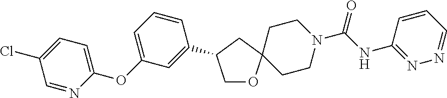

To a mixture of tert-butyl (3S)-3-(3-hydroxyphenyl)-1-oxa-8-azaspiro[4.5]decane-8-carboxylate (340 mg, 1.02 mmol) and cesium carbonate (710 mg, 2.18 mmol) in DMF (3.5 mL) was added 2,5-dichloropyridine (151 mg, 1.02 mmol) and stirred at 90° C. over two nights. Added water (20 mL) and extracted with ethyl acetate (2×10 mL). The organic was dried over magnesium sulfate, filtered, and concentrated to give an oil. The oil was dissolved in methylene chloride (5 mL) and treated with 4 N HCl/dioxane (2 mL). After stirring for 3 h at room temp, the reaction mixture was evaporated overnight. To the crude residue and phenyl pyridazin-3-ylcarbamate (263 mg, 1.22 mmol) in acetonitrile (5 mL) was added DIEA (0.711 mL, 4.08 mmol). The resulting solution was stirred 3 h and then concentrated under nitrogen. The residue was dissolved in MeOH and purified by reverse phase HPLC (5-95% acetonitrile/water/0.05% TFA). The pure fractions were concentrated and partitioned between ethyl acetate and satd bicarbonate solution. The organic phase was dried over sodium sulfate, filtered, and concentrated to give an oil. The oil was dissolved in EA and purified by flash chromatography (2-40% IPA/EA) to give the title compound as an oil (330 mg, 63%). m/z 466 (MH⁺). ¹H NMR (400 MHz, CDCl₃) δ ppm 1.62-1.93 (m, 6H), 2.29 (dd, J=12.6, 7.9 Hz, 1H), 3.42-3.61 (m, 3H), 3.79-3.89 (m, 3H), 4.26 (t, J=8.0 Hz, 1H), 6.90 (d, J=8.5 Hz, 1H), 6.97-7.04 (m, 2H), 7.10 (d, J=7.9 Hz, 1H), 7.36 (t, J=7.7 Hz, 1H), 7.41 (dd, J=9.0, 4.6 Hz, 1H), 7.66 (dd, J=8.5, 2.7 Hz, 1H), 8.13 (d, J=2.7 Hz, 1H), 8.30 (d, J=8.9 Hz, 1H), 8.83 (d, J=4.1 Hz, 1H).

### Phenyl (3,4-dimethylisoxazol-5-yl)carbamate

Method A. 5-amino-3,4-dimethylisoxazole (Aldrich, 5.0 g, 40 mmol; CAS# 19947-75-2) was dissolved in acetonitrile (75 mL) and cooled to 0° C. Phenyl chloroformate (5.91 mL, 46.8 mmol) dissolved in acetonitrile (50 mL) was then added slowly followed immediately by 1,8-bis(dimethylamino)naphthalene (Proton Sponge®, Aldrich; 9.56 g, 44.6 mmol) in acetonitrile (25 mL). The reaction was warmed to RT and stirred for 48 hours. The reaction was quenched with water (100 mL) and extracted with ethyl acetate (2×250 mL). The organics were dried with magnesium sulfate and concentrated to give a crude yellow oil. The crude product was purified by flash chromatography (ethyl acetate/heptane) to give the title compound as a white solid (9.02 g, 38.84 mmol, 90%). ¹H NMR (400 MHz, DMSO-d₆) δ ppm 10.70 (br. s., 1H), 7.40-7.47 (m, 2H), 7.26-7.30 (m, 1H), 7.21-7.25 (m, 2H), 2.16 (s, 3H), 1.86 (s, 3H). m/z 233 (MH⁺).

Method B. A three necked 5 L RB flask equipped with nitrogen bubbler and thermo pocket, was purged well with nitrogen for 20 min at RT. Phenyl chloroformate (120.1 mL, 0.93 mol) in acetonitrile (1 L) was added to the stirred solution of 5-amino-3,4-dimethylisoxazole (AKSCIENTIFIC; 100 g, 0.89 mol) in acetonitrile (1.5 L) at <10° C. over 38 min under nitrogen followed by addition of 1,8-bis(dimethylamino) naphthalene (Proton Sponge®, Aldrich; 189.9 g, 0.886 mol) portionwise over 27 min. After stirring at <10° C. for 10 min, resulting reaction mixture was stirred at RT for 112 h under nitrogen atmosphere. After completion of the reaction (monitored by TLC, 30% EtOAc/hexane), the solid was filtered off and washed with EtOAc (2×375 mL). The filtrate was diluted with water (1.25 L) and EtOAc (2.5 L) and shook well. The layers were separated and the aqueous layer was back extracted with EtOAc (1.25 L). The organic layers were dried over sodium sulfate and concentrated under reduced pressure at 28° C. to afford residue as a greenish oil. The residue was dissolved in EtOAc (2.5 L), washed with water (3×600 mL), dried over sodium sulfate, and concentrated under reduced pressure at 28° C. to afford compound the title compound (207 g) as greenish yellow solid, which was dissolved in EtOAc (1 L), stirred with charcoal (20.7 g) for 30 min at RT and filtered through celite, washing the celite with EtOAc. Upon concentration of filtrate under reduced pressure at 28° C. the title compound was obtained as white solid which was dissolved in EtOAc (1080 mL) and heptane (1080 mL) and stirred for 10 min at RT. Crystallization initiated upon stirring. To this was added heptane (2220 mL) over a period of 30 min. The suspension was stirred for 30 min at RT. The solid was filtered and washed with heptane (2×150 mL) to afford the first crop of the title compound as a white crystalline solid (105 g). The mother liquor was concentrated under reduced pressure at 28° C. to afford 100 g crude product, which was recrystallized from EtOAc/heptane using the above crystallization method to obtain another 48 g of title compound as second crop. The total yield was 153 g (74%). ¹H NMR (acetone-d₆, 400 MHz) δ ppm 9.5 (1H, bs), 7.44-7.40 (2H, m), 7.26 (1H, d, J=7.04 Hz), 7.22 (1H, d, J=8.64 Hz), 2.18 (3H, s), 1.92 (3H, s).

### Examples 13-28

Step 1. A 0.4 M stock solution of tert-butyl (3RS)-3-bromo-1-oxa-8-azaspiro[4.5]decane-8-carboxylate in anhydrous isopropanol (0.5 mL, 0.200 mmol) and a 0.024 M stock solution of trans 2-aminocyclohexanol in isopropanol (0.5 mL, 0.012 mmol, 0.06 equiv) were added to vials containing sodium hexamethyldisilazide (0.400 mmol, 2 equiv), nickel iodide (0.012 mmol, 0.06 equiv), and the appropriate aryl boronic acid (0.400 mmol, 2 equiv). The vials were flushed with nitrogen, capped, and shook at 70° C. overnight. The reaction mixtures were concentrated under vacuum to give the crude tert-butyl carbamate derivatives. Step 2. The residues were dissolved in dichloromethane (1.2 mL) and treated with 4 N HCl in dioxane (0.8 mL). After shaking for 2 h, the reaction mixtures were concentrated under vacuum to give the crude amine hydrochloride salt derivatives. Step 3. The crude amine hydrochloride salt residues were dissolved in acetonitrile (2.0 mL) and split into two separate vials (1.0 mL, 0.1 mmol each). To the solutions was added diisopropylethylamine (0.17 mL, 1.0 mmol, 10 equiv) followed by a solution of phenyl (3,4-dimethylisoxazol-5-yl)carbamate (0.120 mmol in acetonitrile, 1.2 equiv). After shaking overnight at RT, the reactions were concentrated under vacuum, diluted in DMSO (1.5 mL), filtered through celite, and purified by reverse phase HPLC (acetonitrile/water/0.05% trifluoroacetic acid) to give racemic Examples 13-28. The purified compounds were analyzed by LCMS (Phenomenex Gemini C18 4.6×50 mm 5 μm; 0.04% Formic Acid, 0.01% TFA/MeCN).

| Ex. | Name | t_{R} (min) | m/z (MH⁺) |
|---|---|---|---|
| 13 | (3RS)-N-(3,4-dimethylisoxazol-5-yl)-3-[3-(trifluoromethoxy)phenyl]-1- | 1.98 | 440.25 |
| | oxa-8-azaspiro[4.5]decane-8-carboxamide | | |
| 14 | (3RS)-3-(3-chlorophenyl)-N-(3,4-dimethylisoxazol-5-yl)-1-oxa-8- | 1.84 | 390.45 |
| | azaspiro[4.5]decane-8-carboxamide | | |
| 15 | (3RS)-3-(3,4-dichlorophenyl)-N-(3,4-dimethylisoxazol-5-yl)-1-oxa-8- | 1.85 | 424.15 |
| | azaspiro[4.5]decane-8-carboxamide | | |
| 16 | (3RS)-3-(3-chloro-5-fluorophenyl)-N-(3,4-dimethylisoxazol-5-yl)-1-oxa-8- | 1.77 | 408.35 |
| | azaspiro[4.5]decane-8-carboxamide | | |
| 17 | (3RS)-N-(3,4-dimethylisoxazol-5-yl)-3-{4-[(4-fluorobenzyl)oxy]phenyl}-1- | 1.97 | 480.25 |
| | oxa-8-azaspiro[4.5]decane-8-carboxamide | | |
| 18 | (3RS)-3-{3-chloro-4-[(2-chlorobenzyl)oxy]phenyl}-N-(3,4- | 2.21 | 530.15 |
| | dimethylisoxazol-5-yl)-1-oxa-8-azaspiro[4.5]decane-8-carboxamide | | |
| 19 | (3RS)-3-{3-chloro-4-[(4-fluorobenzyl)oxy]phenyl}-N-(3,4- | 2.07 | 514.25 |
| | dimethylisoxazol-5-yl)-1-oxa-8-azaspiro[4.5]decane-8-carboxamide | | |
| 20 | (3RS)-3-[4-(benzyloxy)-3-chlorophenyl]-N-(3,4-dimethylisoxazol-5-yl)-1- | 2.07 | 496.55 |
| | oxa-8-azaspiro[4.5]decane-8-carboxamide | | |
| 21 | (3RS)-3-{3-chloro-4-[(3-chlorobenzyl)oxy]phenyl}-N-(3,4- | 2.21 | 530.35 |
| | dimethylisoxazol-5-yl)-1-oxa-8-azaspiro[4.5]decane-8-carboxamide | | |
| 22 | (3RS)-N-(3,4-dimethylisoxazol-5-yl)-3-{4-[(3-fluorobenzyl)oxy]phenyl}-1- | 1.99 | 480.15 |
| | oxa-8-azaspiro[4.5]decane-8-carboxamide | | |
| 23 | (3RS)-3-{4-[(4-chlorobenzyl)oxy]phenyl}-N-(3,4-dimethylisoxazol-5-yl)-1- | 2.11 | 496.45 |
| | oxa-8-azaspiro[4.5]decane-8-carboxamide | | |
| 24 | (3RS)-3-(1,3-benzodioxol-5-yl)-N-(3,4-dimethylisoxazol-5-yl)-1-oxa-8- | 1.61 | 400.25 |
| | azaspiro[4.5]decane-8-carboxamide | | |
| 25 | (3RS)-N-(3,4-dimethylisoxazol-5-yl)-3-(2-naphthyl)-1-oxa-8- | 1.79 | 406.45 |
| | azaspiro[4.5]decane-8-carboxamide | | |
| 26 | (3RS)-3-(4-chloro-3-fluorophenyl)-N-(3,4-dimethylisoxazol-5-yl)-1-oxa-8- | 1.78 | 408.35 |
| | azaspiro[4.5]decane-8-carboxamide | | |
| 27 | (3RS)-3-(4-chloro-2-fluorophenyl)-N-(3,4-dimethylisoxazol-5-yl)-1-oxa-8- | 1.81 | 408.35 |
| | azaspiro[4.5]decane-8-carboxamide | | |
| 28 | (3RS)-3-(3-chloro-4-fluorophenyl)-N-(3,4-dimethylisoxazol-5-yl)-1-oxa-8- | 1.73 | 408.35 |
| | azaspiro[4.5]decane-8-carboxamide | | |

### tert-butyl (3RS)-3-[4-(benzyloxy)phenyl]-1-oxa-8-azaspiro[4.5]decane-8-carboxylate

To a solution of tert-butyl 3-oxo-1-oxa-8-azaspiro[4.5]decane-8-carboxylate (10 g, 39.2 mmol) in MeTHF (150 mL) at 0° C. was added a solution of 4-benzyloxyphenylmagnesium bromide (78.3 mL, 78.3 mmol, 1 M in THF; Aldrich) dropwise via an addition funnel. After 30 min at 0° C., the reaction was warmed to RT and stirred for 2 hours. The reaction was quenched with saturated ammonium chloride (200 mL) and the layers were separated. The aqueous was extracted with ethyl acetate (100 mL). The combined organic layers were dried over magnesium sulfate, filtered, and concentrated to give an oil (˜28 g). A solution of the oil and triethylsilane (31.3 mL, 196 mmol) in methylene chloride (200 mL) was treated with borontrifluoride diethyl etherate (9.68 mL, 78.4 mmol) and trifluoroacetic acid (14.6 mL, 196 mmol) at 0° C. After stirring overnight at RT, the reaction mixture was concentrated. The residue was dissolved in ethyl acetate (200 mL) and the organic was treated with 2.5 N NaOH (100 mL) and water (100 mL). The layers were separated and the aqueous layer was extracted with ethyl acetate (2×100 mL). The combined organic layers were dried over magnesium sulfate, filtered, and concentrated to give an oil. The oil was treated with anhydrous diethyl ether (200 mL) and the resulting mixture was treated with 2 N HCl/diethyl ether (25 mL) to give a ppt. The mixture was decanted and the residue washed with diethyl ether (2×30 mL) to give a brown gum. To the gum in THF (50 mL) was added Boc anhydride (5.99 g, 27.4 mmol), DMAP (cat.), and DIEA (10.2 mL, 58.8 mmol) and the mixture was stirred overnight. The mixture was treated with water (200 mL) and 1N HCl (100 mL) and extracted with ethyl acetate (2×100 mL). The organic was dried over magnesium sulfate, filtered and evaporated to give an oil. The oil was purified by flash chromatography (5-40% EA/heptane) to give the title compound as an oil (6.48 g, 39%). m/z 368 (MH⁺ minus tBu).

### tert-butyl (3RS)-3-(4-hydroxyphenyl)-1-oxa-8-azaspiro[4.5]decane-8-carboxylate

Method A. To a solution of tert-butyl (3RS)-3-[4-(benzyloxy)phenyl]-1-oxa-8-azaspiro[4.5]decane-8-carboxylate (6.48 g, 15.3 mmol) in 50% EA/methanol (50 mL) was added 10% Pd/C (200 mg, CAS#7440-05-3) catalyst under nitrogen, and the mixture was stirred under 40 psi of hydrogen over several nights. Very little progression of the reaction so the mixture was filtered through a plug of celite and the resulting filtrate was treated with 10% Pd/C (1.3 g, CAS#7440-05-3) catalyst under nitrogen. The mixture was stirred under 40 psi of hydrogen overnight. The mixture was filtered through a plug of celite and the resulting filtrate evaporated to give the title compound as a foam (5.07 g, 99%). m/z 356 (MNa⁺). ¹H NMR (400 MHz, DMSO-d₆) δ ppm 1.39 (s, 9H), 1.44-1.53 (m, 1H), 1.53-1.64 (m, 3H), 1.63-1.72 (m, 1H), 2.18 (dd, J=12.29, 7.85 Hz, 1H), 3.19-3.31 (m, 2H), 3.35-3.49 (m, 3H), 3.49-3.57 (m, 1H), 3.98-4.07 (m, 1H), 6.68 (d, J=8.53 Hz, 2H), 7.06 (d, J=8.19 Hz, 2H), 9.22 (s, 1H).

Method B. The tert-butyl (3RS)-3-{[(trifluoromethyl)sulfonyl]oxy}-1-oxa-8-azaspiro[4.5]dec-3-ene-8-carboxylate (6.0 g, 15 mmol) and 4-hydroxyphenylboronic acid (4.1 g, 30 mmol) were dissolved in DME (100 mL). 2M aqueous Na₂CO₃ (31 mL, 62 mmol) was added, and the resulting mixture was deoxygenated with a stream of Argon. After 10 min, Pd(PPh₃)₄ (512 mg, 0.443 mmol) was added and the mixture was brought to reflux for 30 min. The mixture was partitioned between ethyl acetate and saturated ammonium chloride and dried over sodium sulfate. After filtration, the concentrated crude product was purified by silica gel chromatography with 20% EtOAc in heptane to provide the olefin tert-butyl (3RS)-3-(4-hydroxyphenyl)-1-oxa-8-azaspiro[4.5]dec-3-ene-8-carboxylate (3.93 g) as a white solid. m/z 354 (MNa⁺). The olefin (3.93 g, 11.9 mmol) was dissolved in methanol (100 mL), treated with 10% Pd/C (400 mg), and slurried under an atmosphere of hydrogen (40 psi) for 1 h. The reaction mixture was purged with nitrogen and filtered through a pad of celite. The filtrate was concentrated to give the title compound as a white foam (3.8 g, 76% yield over two steps).

### Examples 29-32

A 0.125 M stock solution of tert-butyl (3RS)-3-(4-hydroxyphenyl)-1-oxa-8-azaspiro[4.5]decane-8-carboxylate in dichloromethane (1.0 mL, 0.125 mmol) was added to each vial containing the appropriate alcohol (0.150 mmol). A 0.1 M PS-PPh₃ suspension in dichloromethane (2 mL) and a 0.2 M DBAD solution in dichloromethane (1 mL) were added to each vial. The vials were capped and shaken at RT for 24 hours. The reaction mixtures were filtered and concentrated. The resultant residues were treated with 25% trifluoroacetic acid/dichloromethane (1.5 mL) and shaken for 2 hours at RT. The reactions were concentrated and the resultant residues were treated with a 0.0625 M solution of phenyl (3,4-dimethylisoxazol-5-yl)carbamate in acetonitrile (2 mL) followed by triethylamine (0.250 mL). After shaking overnight at room temperature, the vials were concentrated. The residues were dissolved in DMSO (1.5 mL) and purified by reverse phase HPLC (acetonitrile/water/0.01% trifluoroacetic acid/0.04% formic acid) to give racemic Examples 29-32. The purified compounds were analyzed by LCMS (Phenomenex Gemini C18 4.6×50 mm 5 μm; 0.04% Formic Acid, 0.01% TFA/MeCN).

| | | t_{R} | |
|---|---|---|---|
| Ex. | Name | (min) | m/z (MH⁺) |
| 29 | (3RS)-N-(3,4-dimethylisoxazol-5-yl)-3-(4-{[6- | 1.95 | 531.55 |
| | (trifluoromethyl)pyridin-2-yl]methoxy}phenyl)- | | |
| | 1-oxa-8-azaspiro[4.5]decane-8-carboxamide | | |
| 30 | (3RS)-3-{4-[(3,4-difluorobenzyl)oxy]phenyl}- | 2.04 | 498.55 |
| | N-(3,4-dimethylisoxazol-5-yl)-1-oxa-8- | | |
| | azaspiro[4.5]decane-8-carboxamide | | |
| 31 | (3RS)-3-{4-[(2,5-difluorobenzyl)oxy]phenyl}- | 2.03 | 498.55 |
| | N-(3,4-dimethylisoxazol-5-yl)-1-oxa-8- | | |
| | azaspiro[4.5]decane-8-carboxamide | | |
| 32 | (3RS)-N-(3,4-dimethylisoxazol-5-yl)-3-(4-{[5- | 1.92 | 531.55 |
| | (trifluoromethyl)pyridin-2-yl]methoxy}phenyl)- | | |
| | 1-oxa-8-azaspiro[4.5]decane-8-carboxamide | | |

### (3RS)-3-[3-(trifluoromethoxy)phenyl]-1-oxa-8-azaspiro[4.5]decane hydrochloride

3-(trifluoromethoxy)bromobenzene (16.0 g, 9.91 mmol; CAS # 2252-44-0) was added to a −5° C. soln of isopropylmagnesium chloride lithium chloride complex in THF (50.4 mL, 65.6 mmol; 1.3M soln from Aldrich). The solution was stirred on an ice-salt bath which was allowed to warm to room temperature overnight. The resulting solution was added to tert-butyl 3-oxo-1-oxa-8-azaspiro[4.5]decane-8-carboxylate (8.5 g, 33 mmol) in 2-MeTHF (150 mL) at 0° C. via an addition funnel and the reaction was stirred at RT overnight. More Grignard reagent (prepared from 11.2 mL isopropyl magnesium chloride and 2.3 mL 3-(trifluoromethoxy) bromobenzene) was added to the reaction at 0° C. and the reaction was stirred for 30 min. The reaction was quenched with ammonium chloride (200 mL) and the layers were separated. The aqueous was extracted with ethyl acetate (2×100 mL). The organic layers were dried over magnesium sulfate, filtered, and concentrated to give a crude oil. A solution of the oil and triethylsilane (26.4 mL, 165 mmol) in methylene chloride (150 mL) was treated with borontrifluoride diethyl etherate (8.14 mL, 66.0 mmol) and trifluoroacetic acid (12.3 mL, 165 mmol) at 0° C. After standing overnight in the freezer, the reaction mixture was concentrated. The residue was dissolved in ethyl acetate and water. The aqueous was made basic with 2.5 N NaOH and extracted with ethyl acetate. The combined organic layers were dried over magnesium sulfate, filtered, and concentrated to give an oil. The oil was dissolved in MeOH (60 mL) and treated with 10% Pd/C (0.67 g), acetic acid (5 mL), and stirred under hydrogen (20 psi) over several days to remove alkene by-product. Added another portion of 10% Pd/C (0.50 g) and stirred under hydrogen (20 psi) overnight. Another portion of 10% Pd/C (0.20 g) was added to the mixture and stirred under hydrogen (20 psi) overnight. The mixture was filtered through a pad of celite and the filtrate evaporated to give an oil. The oil was treated with water (100 mL) and 2.5 N NaOH (80 mL) and extracted with ethyl acetate (2×100 mL). The organic layer was dried over magnesium sulfate, filtered and evaporated to give an orange oil. The oil was treated with anhydrous diethyl ether (50 mL) and the resulting solution was treated with HCl (15 mL, 2N in diethyl ether) to give a ppt. The precipitate was collected by vacuum filtration and washed with diethyl ether (2×20 mL) to give the title compound as an off white solid (4.62 g, 41%). m/z 302 (MH⁺).

### tert-butyl (3RS)-3-[3-(trifluoromethoxy)phenyl]-1-oxa-8-azaspiro[4.5]decane-8-carboxylate

To (3RS)-3-[3-(trifluoromethoxy)phenyl]-1-oxa-8-azaspiro[4.5]decane hydrochloride (4.04 g, 12.0 mmol) in THF (23 mL) was added Boc anhydride (2.92 g, 13.2 mmol), DMAP (148 mg, 1.2 mmol), and DIEA (4.17 mL, 23.9 mmol) and the mixture was stirred for 2 hrs. The mixture was treated with EA (75 mL), water (50 mL), 1N HCl (20 mL) and extracted with ethyl acetate (2×30 mL). The organic was dried over magnesium sulfate, filtered and evaporated to give an oil. The oil was purified by flash chromatography (0-10% IPA/DCM) to give the title compound as an oil (4.75 g, 99%). m/z 346 (MH⁺ minus tBu).

### tert-butyl 3-[3-(trifluoromethoxy)phenyl]-1-oxa-8-azaspiro[4.5]decane-8-carboxylate Enantiomers 1 and 2

The racemic tert-butyl (3RS)-3-[3-(trifluoromethoxy)phenyl]-1-oxa-8-azaspiro[4.5]decane-8-carboxylate (4.75 g, 11.83 mmol) was separated by chiral SFC on a 30×250 mm ChiralPak AD-H column (15% MeOH/CO₂ @ 70 mL/min; 1 mL injections of a 47.5 mg/mL MeOH solution). The first eluting peak gave Enantiomer 1 of the title compound (2.1 g, 88%; t_{R}=0.66 min (4.6×100 mm Chiralpak AD-H, 20% MeOH/CO₂ at 3 mL/min)) and the second eluting peak gave Enantiomer 2 of the title compound (1.9 g, 80%; t_{R}=0.86 min (4.6×100 mm Chiralpak AD-H, 20% MeOH/CO₂ at 3 ml/min)). m/z 346 (MH⁺ minus tBu).

### 3-[3-(trifluoromethoxy)phenyl]-1-oxa-8-azaspiro[4.5]decane hydrochloride Enantiomer 1

To tert-butyl 3-[3-(trifluoromethoxy)phenyl]-1-oxa-8-azaspiro[4.5]decane-8-carboxylate Enantiomer 1 (2.1 g, 5.20 mmol) was added DCM (13 mL) followed by 4N HCl/dioxane (9 mL). The solution was stirred for 3 hr at RT and then evaporated to give the title compound as a solid (1.70 g, 96%). m/z 302 (MH⁺). ¹H NMR (400 MHz, DMSO-d₆) δ ppm 1.75-1.94 (m, 5H), 2.33 (dd, J=12.46, 7.68 Hz, 1H), 3.09 (br. s., 4H), 3.53-3.71 (m, 2H), 4.16 (t, J=7.51 Hz, 1H), 7.23 (d, J=8.19 Hz, 1H), 7.30 (s, 1H), 7.35 (d, J=7.85 Hz, 1H), 7.46 (t, J=7.85 Hz, 1H), 8.83 (br. s., 2H).

### Example 33

### N-(3,4-dimethylisoxazol-5-yl)-3-[3-(trifluoromethoxy)phenyl]-1-oxa-8-azaspiro[4.5]decane-8-carboxamide Enantiomer 1

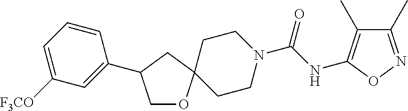

A mixture of 3-[3-(trifluoromethoxy)phenyl]-1-oxa-8-azaspiro[4.5]decane hydrochloride Enantiomer 1 (300 mg, 0.888 mmol), and phenyl (3,4-dimethylisoxazol-5-yl)carbamate (206 mg, 0.888 mmol) in acetonitrile (5 mL) was treated with DIEA (0.619 mL, 3.55 mmol) and stirred at room temperature overnight. The solvent was evaporated and the residue was purified by reverse phase chromatography (5-95% acetonitrile/water/0.05% TFA). The resulting residue was dissolved in EA and washed with 0.25 N NaOH (25 mL). The aqueous layer was then extracted with EA (10 mL) and the combined organics were dried over magnesium sulfate, filtered and evaporated to give an oil. The oil was purified by flash chromatography (0-80% EA/Heptane) to give the title compound as a solid (279 mg, 72%). m/z 440 (MH⁺). ¹H NMR (400 MHz, CDCl₃) δ ppm 1.64-1.74 (m, 1 H), 1.78-1.88 (m, 4H), 1.89 (s, 3H), 2.20 (s, 3H), 2.31 (dd, J=12.63, 8.19 Hz, 1H), 3.37-3.50 (m, 2H), 3.50-3.62 (m, 1H), 3.69-3.78 (m, 2H), 3.82 (t, J=8.88 Hz, 1H), 4.26 (t, J=8.19 Hz, 1H), 6.58 (s, 1H), 7.07-7.14 (m, 2H), 7.18 (d, J=7.85 Hz, 1H), 7.35 (t, J=7.68 Hz, 1H).

### 3-[3-(trifluoromethoxy)phenyl]-1-oxa-8-azaspiro[4.5]decane hydrochloride Enantiomer 2

The title compound was prepared from tert-butyl 3-[3-(trifluoromethoxy)phenyl]-1-oxa-8-azaspiro[4.5]decane-8-carboxylate Enantiomer 2 (1.9 g, 4.70 mmol) as described for 3-[3-(trifluoromethoxy)phenyl]-1-oxa-8-azaspiro[4.5]decane hydrochloride Enantiomer 1 to give the title compound as a solid (1.64 g, quant.). m/z 302 (MH⁺). ¹H NMR (400 MHz, DMSO-d₆) δ ppm 1.75-1.95 (m, 5H), 2.32 (dd, J=12.46, 7.68 Hz, 1H), 2.96-3.19 (m, 4H), 3.53-3.71 (m, 2H), 4.15 (t, J=7.34 Hz, 1H), 7.22 (d, J=8.19 Hz, 1H), 7.30 (s, 1H), 7.35 (d, J=7.85 Hz, 1H), 7.45 (t, J=8.02 Hz, 1H), 8.93 (br. s., 2H).

### Example 34

### N-(3,4-dimethylisoxazol-5-yl)-3-[3-(trifluoromethoxy)phenyl]-1-oxa-8-azaspiro[4.5]decane-8-carboxamide Enantiomer 2

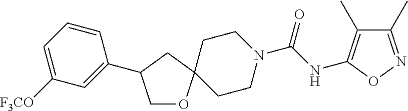

The title compound was prepared from 3-[3-(trifluoromethoxy)phenyl]-1-oxa-8-azaspiro[4.5]decane hydrochloride Enantiomer 2 (300 mg, 0.888 mmol,) as described for Example 33 to give the title compound as a solid (282 mg, 72%). m/z 440 (MH⁺). ¹H NMR (400 MHz, CDCl₃) δ ppm 1.64-1.73 (m, 1H), 1.79-1.88 (m, 4H), 1.89 (s, 3H), 2.20 (s, 3H), 2.31 (dd, J=12.63, 8.19 Hz, 1H), 3.38-3.50 (m, 2H), 3.51-3.61 (m, 1 H), 3.69-3.78 (m, 2H), 3.81 (t, J=8.88 Hz, 1H), 4.22-4.29 (m, 1H), 6.58 (s, 1H), 7.07-7.13 (m, 2H), 7.18 (d, J=7.51 Hz, 1H), 7.35 (t, J=7.68 Hz, 1H).

### (3RS)-3-(3-chlorophenyl)-1-oxa-8-azaspiro[4.5]decane hydrochloride

To a solution of tert-butyl 3-oxo-1-oxa-8-azaspiro[4.5]decane-8-carboxylate (8.65 g, 33.9 mmol) in 2-MeTHF (150 mL) at 0° C. was added 3-chlorophenylmagnesium bromide (0.5 M in THF, 97%, 136 mL, 67.8 mmol; Aldrich) dropwise. After 2 hours the reaction was treated with more 3-chlorophenylmagnesium bromide (20 mL, 9.7 mmol) at 0° C. After stirring overnight at RT, the reaction was quenched with saturated ammonium chloride (200 mL) and extracted with ethyl acetate (2×100 mL). The organic layers were dried over magnesium sulfate, filtered, and concentrated to give the crude alcohol as an oil. A solution of the crude alcohol and triethylsilane (27.1 mL, 170 mmol, 5 equiv) in methylene chloride (150 mL) was treated with borontrifluoride diethyl etherate (8.37 mL, 67.8 mmol, 2 equiv) and trifluoroacetic acid (12.6 mL, 170 mmol, 5 equiv) at 0° C. The reaction was stirred for 1 hr at 0° C. and then stored in the freezer overnight. The reaction was warmed to room temperature, treated with more trifluoroacetic acid (12 mL, 162 mmol) and then 3 hours later more triethylsilane (13 mL, 81.3 mmol), borontrifluoride diethyl etherate (4 mL, 32.4 mmol) and trifluoroacetic acid (30 mL, 404 mmol) were added. After stirring over the weekend at RT, the reaction mixture was concentrated. The residue was dissolved in ethyl acetate and the organic made basic with 2.5 N NaOH and water. The layers were separated and the aqueous layer was extracted with ethyl acetate. The combined organic layers were dried over magnesium sulfate, filtered, and concentrated to give 9 g of an oil. To the oil in MeOH (60 mL) was added acetic acid (5 mL) followed by 10% Pd/C (0.67 g) under nitrogen and the mixture was stirred under 20 psi hydrogen for several days. The reaction mixture was filtered and then made basic with water (100 mL) and 2.5N NaOH (70 mL). The mixture was extracted with ethyl acetate (2×100 mL), dried over magnesium sulfate, filtered and evaporated to give 6.9 g of an orange oil. To the oil in diethyl ether (50 mL) was added 2 N HCl/diethyl ether (15 mL) and the resulting ppt was collected by vacuum filtration to give the title compound as a brown solid (4.37 g, 45%). m/z 252 (MH⁺). ¹H NMR (400 MHz, DMSO-d₆) δ ppm 1.75-1.98 (m, 5H), 2.30 (dd, J=12.63, 7.85 Hz, 1H), 2.97-3.16 (m, 4H), 3.48-3.60 (m, 1 H), 3.65 (t, J=8.88 Hz, 1H), 4.14 (t, J=7.68 Hz, 1H), 7.24-7.40 (m, 4H), 8.89 (br. s., 2H).

### tert-butyl (3RS)-3-(3-chlorophenyl)-1-oxa-8-azaspiro[4.5]decane-8-carboxylate

To a solution of (3RS)-3-(3-chlorophenyl)-1-oxa-8-azaspiro[4.5]decane hydrochloride (3.67 g, 12.7 mmol) in THF (25 mL) was added Boc anhydride (3.1 g, 14.0 mmol), DMAP (157 mg, 0.1 mmol), and DIEA (4.44 mL, 25.5 mmol) and the mixture was stirred for 2 hours. The reaction was diluted with ethyl acetate (75 mL) and treated with water (50 mL) and 1N HCl (20 mL). The layers were separated and the aqueous was extracted with ethyl acetate (2×30 mL). The combined organic was dried over magnesium sulfate, filtered and evaporated to give an oil. The oil was purified by flash chromatography (0-10% IPA/DCM) to give the title compound as an oil (4.7 g, 100%). m/z 296 (MH⁺ minus tBu).

### tert-butyl 3-(3-chlorophenyl)-1-oxa-8-azaspiro[4.5]decane-8-carboxylate Enantiomers 1 and 2

The racemate tert-butyl (3RS)-3-(3-chlorophenyl)-1-oxa-8-azaspiro[4.5]decane-8-carboxylate (4.7 g) was separated by chiral SFC on a 30×250 mm ChiralPak AD-H column (20% MeOH/CO₂ @ 70 mL/min; 2 mL injections of a 45 mg/mL MeOH solution). The first eluting peak gave Enantiomer 1 of the title compound (1.6 g, 68%; t_{R}=1.03 min (4.6×150 mm ChiralPak AD-H, 20% MeOH/CO₂ at 3 mL/min)) and the second eluting peak gave Enantiomer 2 of the title compound as a solid (1.2 g, 51%; t_{R}=2.24 min (4.6×150 mm ChiralPak AD-H, 20% MeOH/CO₂ at 3 mL/min)). Enantiomer 1 was then further purified by chiral SFC on a 30×250 mm ChiralCel OD-H column (20% MeOH/CO₂ @ 70 mL/min; 2 mL injections of a 16 mg/mL 50% DCM/MeOH solution) to give Enantiomer 1 of the title compound as a solid (1.2 g, 51%). m/z 296 (MH⁺ minus tBu).

### 3-(3-chlorophenyl)-1-oxa-8-azaspiro[4.5]decane hydrochloride Enantiomer 1

A solution of tert-butyl 3-(3-chlorophenyl)-1-oxa-8-azaspiro[4.5]decane-8-carboxylate Enantiomer 1 (1.2 g, 3.4 mmol) in DCM (13 mL) was treated with 4 N HCl/dioxane (4 mL). The solution was stirred for 3 hr at RT and then evaporated to give the title compound as a solid (1.08 g, quant.). m/z 252 (MH⁺). ¹H NMR (400 MHz, DMSO-d₆) δ ppm 1.71-1.96 (m, 5H), 2.30 (dd, J=12.63, 7.85 Hz, 1H), 2.96-3.19 (m, 4H), 3.49-3.61 (m, 1H), 3.62-3.71 (m, 1H), 4.14 (t, J=7.68 Hz, 1H), 7.22-7.43 (m, 4H), 8.87 (br. s., 2H).

### Example 35

### 3-(3-chlorophenol)-N-(3,4-dimethylisoxazol-5-yl)-1-oxa-8-azaspiro[4.5]decane-8-carboxamide Enantiomer 1

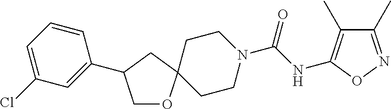

A mixture of 3-(3-chlorophenyl)-1-oxa-8-azaspiro[4.5]decane hydrochloride Enantiomer 1 (300 mg, 0.529 mmol) and phenyl (3,4-dimethylisoxazol-5-yl)carbamate (242 mg, 1.04 mmol) in acetonitrile (5 mL) was treated with DIEA (0.871 mL, 5.0 mmol) and stirred for 3 hrs. The reaction was treated with TFA (0.25 mL) and purified by reverse phase chromatography (5-95% acetonitrile/water/0.05% TFA) to give a solid. The solid was purified by flash chromatography (0-20% IPA/EA) to give the title compound as a solid (292 mg, 72%). m/z 390 (MH⁺). ¹H NMR (400 MHz, CDCl₃) δ ppm 1.63-1.72 (m, 1H), 1.74-1.88 (m, 4H), 1.89 (s, 3 H), 2.20 (s, 3H), 2.28 (dd, J=12.63, 8.19 Hz, 1H), 3.37-3.57 (m, 3H), 3.69-3.76 (m, 2H), 3.80 (t, J=8.88 Hz, 1H), 4.23 (t, J=8.19 Hz, 1H), 6.63 (s, 1H), 7.13 (d, J=7.17 Hz, 1H), 7.20-7.29 (m, 3H).

### 3-(3-chlorophenyl)-1-oxa-8-azaspiro[4.5]decane hydrochloride Enantiomer 2

The title compound was prepared from tert-butyl 3-(3-chlorophenyl)-1-oxa-8-azaspiro[4.5]decane-8-carboxylate Enantiomer 2 (1.2 g, 3.4 mmol) as described for 3-(3-chlorophenyl)-1-oxa-8-azaspiro[4.5]decane hydrochloride Enantiomer 1 to give the title compound as a solid (1.14 g, quant.). m/z 252 (MH⁺). ¹H NMR (400 MHz, DMSO-d₆) δ ppm 1.74-1.95 (m, 5H), 2.30 (dd, J=12.46, 7.68 Hz, 1H), 2.98-3.18 (m, 4H), 3.50-3.61 (m, 1H), 3.60-3.72 (m, 1H), 4.14 (t, J=7.68 Hz, 1H), 7.20-7.43 (m, 4H), 8.88 (br. s., 2H).

### Example 36

### 3-(3-chlorophenyl)-N-(3,4-dimethylisoxazol-5-yl)-1-oxa-8-azaspiro[4.5]decane-8-carboxamide Enantiomer 2

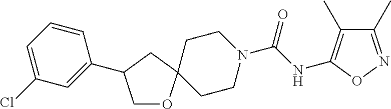

The title compound was prepared from 3-(3-chlorophenyl)-1-oxa-8-azaspiro[4.5]decane hydrochloride Enantiomer 2 (300 mg, 0.529 mmol) as describe for Example 35 to give the title compounds as a solid (282 mg, 70%). m/z 390 (MH⁺). ¹H NMR (400 MHz, CDCl₃) δ ppm 1.62-1.72 (m, 1H), 1.77-1.86 (m, 4H), 1.88 (s, 3H), 2.19 (s, 3H), 2.28 (dd, J=12.63, 8.19 Hz, 1H), 3.37-3.57 (m, 3H), 3.69-3.76 (m, 2H), 3.79 (t, J=8.88 Hz, 1H), 4.23 (t, J=8.19 Hz, 1H), 6.74 (s, 1H), 7.12 (d, J=7.17 Hz, 1H), 7.20-7.27 (m, 3H).

### tert-butyl (3RS)-3-(3,4-dichlorophenyl)-1-oxa-8-azaspiro[4.5]decane-8-carboxylate

To a solution of isopropylmagnesium chloride lithium chloride complex in THF (1.3M soln from Aldrich, 17.8 mL, 23.1 mmol) at −5° C. was added 3,4-dichlorobromobenzene (3.02 mL, 23.5 mmol). The resulting solution was allowed to warm to RT with stirring overnight. To tert-butyl 3-oxo-1-oxa-8-azaspiro[4.5]decane-8-carboxylate (3.00 g, 11.8 mmol) in 2-MeTHF (60 mL) at 0° C. was added a solution of 3,4-dichlorophenylmagnesium bromide (23.1 mmol; generated above). The reaction was allowed to warm to RT overnight. The reaction was quenched with saturated ammonium chloride (100 mL) and extracted with ethyl acetate(2×50 mL). The organic layers were dried over magnesium sulfate, filtered, and concentrated to give the crude alcohol as an oil (˜7 g). A solution of the crude alcohol and triethylsilane (9.43 mL, 59 mmol, 5 equiv) in methylene chloride (75 mL) was treated with borontrifluoride diethyl etherate (2.91 mL, 23.6 mmol, 2 equiv) and trifluoroacetic acid (4.38 mL, 59 mmol, 5 equiv) at 0° C. After stirring overnight at RT, the reaction mixture was concentrated. The residue was dissolved in ethyl acetate and the organic was treated with 2.5 N NaOH (40 mL) and water (50 mL). The layers were separated and the aqueous layer was extracted with ethyl acetate (3×50 mL). The combined organic layers were dried over magnesium sulfate, filtered, and concentrated to give an oil. To the oil in diethyl ether (50 mL) was added 2 N HCl/diethyl ether (7 mL) and the resulting ppt was collected by vacuum filtration to give a white solid (2.34 g). To the solid in THF (25 mL) was added Boc anhydride (1.80 g, 8.26 mmol), DMAP (cat.), and triethylamine (2.47 mL, 17.7 mmol) and the mixture was stirred overnight. The mixture was treated with water (50 mL) and 1N HCl (20 mL) and extracted with ethyl acetate (3×100 mL). The organic was dried over magnesium sulfate, filtered and evaporated to give an oil. The oil was purified by flash chromatography (5-60% EA/heptane) to give the title compound as a solid (2.45 g, 54%). m/z 330 (MH⁺ minus tBu). ¹H NMR (400 MHz, CDCl₃) δ ppm 1.44-1.50 (m, 9H), 1.52-1.63 (m, 1H), 1.65-1.81 (m, 4H), 2.26 (dd, J=12.63, 8.19 Hz, 1H), 3.28-3.41 (m, 2H), 3.41-3.53 (m, 1 H), 3.63 (br. s., 2H), 3.75 (t, J=8.70 Hz, 1H), 4.16-4.24 (m, 1H), 7.08 (dd, J=8.19, 2.05 Hz, 1H), 7.33 (d, J=2.05 Hz, 1H), 7.38 (d, J=8.53 Hz, 1H).

### tert-butyl 3-(3,4-dichlorophenyl)-1-oxa-8-azaspiro[4.5]decane-8-carboxylate Enantiomers 1 and 2

The racemic tert-butyl (3RS)-3-(3,4-dichlorophenyl)-1-oxa-8-azaspiro[4.5]decane-8-carboxylate (2.45 g) was separated by chiral SFC on a 30×250 mm ChiralPak AD-H column (20% MeOH/CO₂ @ 70 mL/min; 1 mL injections of a 49 mg/mL MeOH solution). The first eluting peak gave Enantiomer 1 of the title compound (0.8 g, 65%; t_{R}=1.11 min (4.6×150 mm ChiralPak AD-H, 20% MeOH/CO₂ at 3 mL/min)) and the second eluting peak gave Enantiomer 2 of the title compound (1.0 g, 81%; t_{R}=1.37 min (4.6×150 mm ChiralPak AD-H, 20% MeOH/CO₂ at 3 ml/min)). m/z 330 (MH⁺ minus tBu).

### 3-(3,4-dichlorophenyl)-1-oxa-8-azaspiro[4.5]decane hydrochloride Enantiomer 1

In a vial tert-butyl 3-(3,4-dichlorophenyl)-1-oxa-8-azaspiro[4.5]decane-8-carboxylate (0.8 g, 2 mmol,) in DCM (3 mL) was treated with 4 N HCl/dioxane (3 mL). The solution was stirred for 3 hr at RT and then evaporated to give the title compound as a solid (0.788 g, quant.). m/z 286 (MH⁺).

### Example 37

### 3-(3,4-dichlorophenyl)-N-(3,4-dimethylisoxazol-5-yl)-1-oxa-8-azaspiro[4.5]decane-8-carboxamide Enantiomer 1

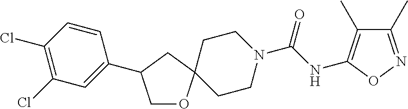

A mixture of 3-(3,4-dichlorophenyl)-1-oxa-8-azaspiro[4.5]decane hydrochloride Enantiomer 1 (250 mg, 0.775 mmol) and phenyl (3,4-dimethylisoxazol-5-yl)carbamate (180 mg, 0.775 mmol) in acetonitrile (5 mL) was treated with DIEA (0.80 mL, 5.0 mmol) and stirred over the weekend. The reaction was evaporated and purified by reverse phase chromatography (20-95% acetonitrile/water/0.05% TFA) to give a solid. The solid was dissolved in EA (100 mL) and washed with 0.25 N NaOH (2×50 mL), and brine (50 mL). The combined organics were dried over magnesium sulfate, filtered and evaporated to give the title compound as a solid (156 mg, 47%). m/z 424 (MH⁺). ¹H NMR (400 MHz, DMSO-d₆) δ ppm 1.54-1.72 (m, 4H), 1.74 (s, 3H), 1.79 (dd, J=12.46, 10.41 Hz, 1H), 2.12 (s, 3H), 2.30 (dd, J=12.63, 7.85 Hz, 1H), 3.35-3.46 (m, 2H), 3.50-3.62 (m, 3H), 3.66 (t, J=8.88 Hz, 1H), 4.13 (t, J=7.68 Hz, 1H), 7.33 (dd, J=8.53, 2.05 Hz, 1H), 7.55-7.62 (m, 2 H), 9.12 (s, 1H).

### 3-(3,4-dichlorophenyl)-1-oxa-8-azaspiro[4.5]decane hydrochloride Enantiomer 2

The title compound was prepared from tert-butyl 3-(3,4-dichlorophenyl)-1-oxa-8-azaspiro[4.5]decane-8-carboxylate (1.0 g, 2.6 mmol) in DCM (3 mL) as described for 3-(3,4-dichlorophenyl)-1-oxa-8-azaspiro[4.5]decane hydrochloride Enantiomer 1 to give the title compound as a solid (0.914 g, quant.). m/z 286 (MH⁺).

### Example 38

### 3-(3,4-dichlorophenyl)-N-(3,4-dimethylisoxazol-5-yl)-1-oxa-8-azaspiro[4.5]decane-8-carboxamide Enantiomer 2

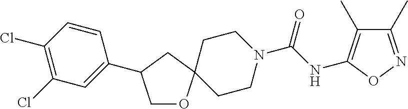

The title compound was prepared from 3-(3,4-dichlorophenyl)-1-oxa-8-azaspiro[4.5]decane hydrochloride Enantiomer 2 (250 mg, 0.775 mmol) as described for Example 37 to give the title compound as a solid (190 mg, 57%). m/z 424 (MH⁺). ¹H NMR (400 MHz, DMSO-d₆) δ ppm 1.54-1.72 (m, 4H), 1.74 (s, 3H), 1.79 (dd, J=12.46, 10.41 Hz, 1H), 2.12 (s, 3H), 2.30 (dd, J=12.63, 7.85 Hz, 1H), 3.35-3.46 (m, 2H), 3.50-3.62 (m, 3H), 3.66 (t, J=8.88 Hz, 1H), 4.13 (t, J=7.68 Hz, 1H), 7.33 (dd, J=8.53, 2.05 Hz, 1H), 7.55-7.62 (m, 2H), 9.12 (s, 1H).

### tert-butyl (3RS)-3-(3-chloro-5-fluorophenyl)-1-oxa-8-azaspiro[4.5]decane-8-carboxylate

To a solution of tert-butyl 3-oxo-1-oxa-8-azaspiro[4.5]decane-8-carboxylate (3.01 g, 11.8 mmol) in 2-MeTHF (100 mL) at 0° C. was added 3-chloro-5-fluorophenylmagnesium bromide (0.5 M in THF, 25 mL, 12.5 mmol; Aldrich) dropwise. After 30 min the reaction was warmed to RT for 2 hrs, and then stored in the freezer for 2 days. More 3-chloro-5-fluorophenylmagnesium bromide (27 mL, 13.5 mmol) was added at 0° C. After stirring overnight at RT, the reaction was quenched with saturated ammonium chloride and extracted with ethyl acetate. The organic layers were dried over magnesium sulfate, filtered, and concentrated to give crude tert-butyl (3RS)-3-(3-chloro-5-fluorophenyl)-3-hydroxy-1-oxa-8-azaspiro[4.5]decane-8-carboxylate as an oil. A solution of the crude alcohol and triethylsilane (9.42 mL, 59 mmol, 5 equiv) in methylene chloride (100 mL) was treated with borontrifluoride diethyl etherate (2.91 mL, 23.6 mmol, 2 equiv) and trifluoroacetic acid (4.38 mL, 59 mmol, 5 equiv) at 0° C. After stirring overnight at RT, the reaction mixture was concentrated. The residue was dissolved in ethyl acetate and the organic was treated with 2.5 N NaOH (40 mL) and water (50 mL). The layers were separated and the aqueous layer was extracted with ethyl acetate (3×50 mL). The combined organic layers were dried over magnesium sulfate, filtered, and concentrated to give 3.27 g of an oil. To the oil in 2-MeTHF (25 mL) was added Boc anhydride (1.80 g, 8.26 mmol), DMAP (cat.), and triethylamine (2.47 mL, 17.7 mmol) and the mixture was stirred overnight. The mixture was treated with water (50 mL) and 1N HCl (20 mL) and extracted with ethyl acetate (3×100 mL). The organic was dried over magnesium sulfate, filtered and evaporated to give an oil. The oil was purified by flash chromatography (0-30% EA/heptane) to give the title compound as a solid (0.42 g, 10%). m/z 314 (MH⁺ minus tBu).

### tert-butyl 3-(3-chloro-5-fluorophenyl)-1-oxa-8-azaspiro[4.5]decane-8-carboxylate Enantiomers 1 and 2

The racemic tert-butyl (3RS)-3-(3-chloro-5-fluorophenyl)-1-oxa-8-azaspiro[4.5]decane-8-carboxylate (0.42 g) was separated by chiral SFC on a 30×250 mm ChiralPak AD-H column (15% MeOH/CO₂ @ 70 mL/Min; 1 mL injections of a 21 mg/mL MeOH solution). The first eluting peak gave Enantiomer 1 of the title compound (146 mg, 70%; t_{R}=0.93 min (4.6×150 mm Chiralpak AD-H, 20% MeOH/CO₂ at 3 mL/min)) and the second eluting peak gave Enantiomer 2 of the title compound (144 mg, 69%; t_{R}=1.13 min (4.6×150 mm Chiralpak AD-H, 20% MeOH/CO₂ at 3 ml/min)). m/z 314 (MH⁺ minus tBu).

### 3-(3-chloro-5-fluorophenyl)-1-oxa-8-azaspiro[4.5]decane hydrochloride Enantiomer 1

The title compound was prepared from tert-butyl 3-(3-chloro-5-fluorophenyl)-1-oxa-8-azaspiro[4.5]decane-8-carboxylate Enantiomer 1 (146 mg) as described for (3RS)-3-(3-{[5-(trifluoromethyl)pyridin-2-yl]oxy}phenyl)-1-oxa-8-azaspiro[4.5]decane hydrochloride to give the title compound as a solid (121 mg, quant.). m/z 270 (MH⁺).

### Example 39

### 3-(3-chloro-5-fluorophenyl)-N-(3,4-dimethylisoxazol-5-yl)-1-oxa-8-azaspiro[4.5]decane-8-carboxamide Enantiomer 1

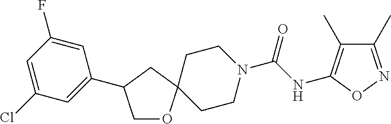

A mixture of 3-(3-chloro-5-fluorophenyl)-1-oxa-8-azaspiro[4.5]decane hydrochloride Enantiomer 1 (121 mg, 0.395 mmol) and phenyl (3,4-dimethylisoxazol-5-yl)carbamate (101 mg, 0.435 mmol) in acetonitrile (3 mL) was treated with DIEA (0.275 mL, 1.58 mmol) and stirred at room temperature overnight. The solvent was evaporated and the residue was purified by reverse phase chromatography (20-95% acetonitrile/water/0.05% TFA). The resulting residue was dissolved in methanol/acetonitrile and eluted through a a StratoSpheres™ PL-HCO₃ MP SPE tube (Polymer Laboratories, Amherst, Mass.) to neutralize any TFA. The eluent was evaporated and the residue was recrystallized from ethyl acetate to give the title compound as a solid (98 mg, 61%). m/z 408 (MH⁺). ¹H NMR (400 MHz, DMSO-d₆) δ ppm 1.52-1.72 (m, 4H), 1.73 (s, 3H), 1.80 (dd, J=12.46, 10.41 Hz, 1H), 2.12 (s, 3H), 2.29 (dd, J=12.63, 7.85 Hz, 1H), 3.34-3.45 (m, 2H), 3.50-3.62 (m, 3 H), 3.62-3.70 (m, 1H), 4.12 (t, J=7.68 Hz, 1H), 7.16-7.24 (m, 1H), 7.24-7.32 (m, 2H), 9.12 (s, 1H).

### 3-(3-chloro-5-fluorophenyl)-1-oxa-8-azaspiro[4.5]decane hydrochloride Enantiomer 2

The title compound was prepared from tert-butyl 3-(3-chloro-5-fluorophenyl)-1-oxa-8-azaspiro[4.5]decane-8-carboxylate Enantiomer 2 (144 mg) as described for (3RS)-3-(3-{[5-(trifluoromethyl)pyridin-2-yl]oxy}phenyl)-1-oxa-8-azaspiro[4.5]decane hydrochloride to give title compound as a solid (121 mg, quant.). m/z 270 (MH⁺).

### Example 40

### 3-(3-chloro-5-fluorophenyl)-N-(3,4-dimethylisoxazol-5-yl)-1-oxa-8-azaspiro[4.5]decane-8-carboxamide Enantiomer 2

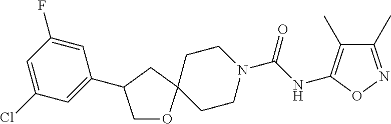

The title compound was prepared from 3-(3-chloro-5-fluorophenyl)-1-oxa-8-azaspiro[4.5]decane hydrochloride Enantiomer 2 (121 mg, 0.395 mmol) as described for Example 39 to give the title compound as a solid (96 mg, 60). m/z 408 (MH⁺). ¹H NMR (400 MHz, DMSO-d₆) δ ppm 1.51-1.72 (m, 4H), 1.73 (s, 3H), 1.80 (dd, J=12.46, 10.41 Hz, 1H), 2.12 (s, 3H), 2.29 (dd, J=12.46, 7.68 Hz, 1H), 3.34-3.44 (m, 2H), 3.50-3.61 (m, 3 H), 3.62-3.70 (m, 1H), 4.12 (t, J=7.51 Hz, 1H), 7.16-7.24 (m, 1H), 7.25-7.31 (m, 2H), 9.12 (s, 1H).

### tert-butyl (3R)-3-(4-hydroxyphenyl)-1-oxa-8-azaspiro[4.5]decane-8-carboxylate and tert-butyl (3S)-3-(4-hydroxyphenyl)-1-oxa-8-azaspiro[4.5]decane-8-carboxylate

The racemic tert-butyl (3RS)-3-(4-hydroxyphenyl)-1-oxa-8-azaspiro[4.5]decane-8-carboxylate (3.2 g, 9.60 mmol) was separated by chiral SFC on a 30×250 mm ChiralPak IA column (20% MeOH/CO₂ @ 70 mL/min; 3 mL injections of a 50 mg/mL MeOH solution). The first eluting peak gave tert-butyl (3R)-3-(4-hydroxyphenyl)-1-oxa-8-azaspiro[4.5]decane-8-carboxylate as a solid (1.09 g, 68%; t_{R}=1.26 min (4.6×100 mm Chiralpak IA, 20% MeOH/CO₂ at 3 mL/min)) and the second eluting peak gave tert-butyl (3S)-3-(4-hydroxyphenyl)-1-oxa-8-azaspiro[4.5]decane-8-carboxylate. The second eluting peak was then further purified by chiral SFC on a 30×250 mm ChiralCel OD-H column (20% EtOH/CO₂ @ 70 mL/min; 3 mL injections of a 50 mg/mL MeOH solution) to give tert-butyl (3S)-3-(4-hydroxyphenyl)-1-oxa-8-azaspiro[4.5]decane-8-carboxylate as a solid (0.89 g, 56%; t_{R}=2.27 min (4.6×100 mm Chiralpak IA, 20% MeOH/CO₂ at 3 ml/min)). m/z 356 (MNa⁺).

### (3R)-3-{4-[(4-fluorobenzyl)oxy]phenyl}-1-oxa-8-azaspiro[4.5]decane hydrochloride

To tert-butyl (3R)-3-(4-hydroxyphenyl)-1-oxa-8-azaspiro[4.5]decane-8-carboxylate (300 mg, 0.90 mmol) was added 4-fluorobenzyl bromide (340 mg, 1.80 mmol), sodium iodide (10 mg, 0.07 mmol), potassium carbonate (700 mg, 5.06 mmol) and acetone (5 mL). The mixture was heated at 80° C. over 2 nights. The mixture was treated with water (˜20 mL) and extracted with ethyl acetate (3×10 mL). The organics were evaporated to give the crude residue. The residues was purified by flash chromatography (10-50% EA/Heptane) to give a solid. The solid in DCM (2 mL) was treated with 4 N HCl/dioxane (2 mL). The solution was stirred for 3 hr at RT and then evaporated to give the title compound as a solid (309 mg, 91%). m/z 342 (MH⁺).

### Example 41

### (3R)—N-(3,4-dimethylisoxazol-5-yl)-3-{4-[(4-fluorobenzyl)oxy]phenyl}-1-oxa-8-azaspiro[4.5]decane-8-carboxamide

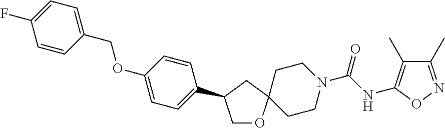

To (3R)-3-{4-[(4-fluorobenzyl)oxy]phenyl}-1-oxa-8-azaspiro[4.5]decane hydrochloride (200 mg, 0.529 mmol) was added phenyl (3,4-dimethylisoxazol-5-yl)carbamate (135 mg, 0.582 mmol), acetonitrile (4 mL), DIEA (0.369 mL, 2.12 mmol) and stirred overnight. The mixture was evaporated and the crude residue was purified by flash chromatography (50-100% EA/Heptane) to give a solid. The solid was recyrstallized from hot EA (˜4 mL) to give the title compound as a solid (208 mg, 82%). m/z 480 (MH⁺). ¹H NMR (400 MHz, DMSO-d₆) δ ppm 1.52-1.81 (m, 8H), 2.12 (s, 3H), 2.23 (d, J=8.88 Hz, 1H), 3.35-3.52 (m, 3H), 3.51-3.66 (m, 3H), 4.09 (t, J=7.85 Hz, 1H), 5.06 (s, 2H), 6.95 (d, J=8.53 Hz, 2H), 7.21 (d, J=3.41 Hz, 4H), 7.48 (t, J=5.97 Hz, 2 H), 9.11 (br. s., 1H).

### (3S)-3-{4-[(4-fluorobenzyl)oxy]phenyl}-1-oxa-8-azaspiro[4.5]decane hydrochloride

The title compound was prepared from tert-butyl (35)-3-(4-hydroxyphenyl)-1-oxa-8-azaspiro[4.5]decane-8-carboxylate (300 mg, 0.90 mmol) as described for (3R)-3-{4-[(4-fluorobenzyl)oxy]phenyl}-1-oxa-8-azaspiro[4.5]decane hydrochloride to give the title compound as a solid (272 mg, 80%). m/z 342 (MH⁺).

### Example 42

### (3S)—N-(3,4-dimethylisoxazol-5-yl)-3-{4-[(4-fluorobenzyl)oxy]phenyl}-1-oxa-8-azaspiro[4.5]decane-8-carboxamide

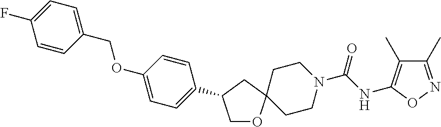

The title compound was prepared from (3R)-3-{4-[(4-fluorobenzyl)oxy]phenyl}-1-oxa-8-azaspiro[4.5]decane hydrochloride (200 mg, 0.529 mmol) as described for Example 41 to give the title compound as a solid (204 mg, 80%). m/z 480 (MH⁺). ¹H NMR (400 MHz, DMSO-d₆) δ ppm 1.51-1.78 (m, 8H), 2.11 (d, J=2.73 Hz, 3 H), 2.25 (dd, J=12.12, 8.36 Hz, 1H), 3.34-3.51 (m, 3H), 3.52-3.65 (m, 3H), 3.98-4.15 (m, 1H), 5.06 (s, 2 H), 6.94 (dd, J=8.70, 2.56 Hz, 2H), 7.14-7.29 (m, 4H), 7.42-7.54 (m, 2H), 9.11 (s, 1H).

### (4-bromo-2-chlorophenoxy)(triisopropyl)silane

Imidazole (1.84 g, 26.50 mmol) was added in one portion to 4-bromo-2-chlorophenol (5.0 g, 24.10 mmol) dissolved in dichloromethane (50 mL) at RT followed by triisopropylsilyl chloride (6.37 mL, 28.9 mmol) and the reaction stirred overnight. The reaction was then quenched with water and the aqueous phase extracted with dichloromethane (2×25 mL), the organics dried with magnesium sulfate and concentrated. The crude product was purified by flash chromatography (ethyl acetetae:heptanes) to give the title compound as a clear oil. (7.85 g, 21.65 mmol, 90%)

### tert-butyl (3RS)-3-{3-chloro-4-[(triisopropylsilyl)oxy]phenyl}-1-oxa-8-azaspiro[4.5]decane-8-carboxylate

To a solution of tert-butyl 3-oxo-1-oxa-8-azaspiro[4.5]decane-8-carboxylate (1.52 g, 5.94 mmol) in THF (25 mL) at 0° C. was added bromo{3-chloro-4-[(triisopropylsilyl)oxy]phenyl}magnesium (prepared from stirring (4-bronno-2-chlorophenoxy)(triisopropyl)silane (3.24 g, 8.90 mmol) and isopropyl magnesium chloride (9.0 mL, 11.70 mmol) in THF (10 mL) at r.t. for 14 hrs). After 1 h, the reaction was quenched with saturated ammonium chloride and extracted with ethyl acetate (2×25 mL). The organic layers were dried over magnesium sulfate, filtered, and concentrated to give the crude alcohol as a pale yellow oil. A solution of the crude alcohol and triethylsilane (4.0 mL, 25.0 mmol) in methylene chloride (50 mL) was treated with borontrifluoride diethyl etherate (1.46 mL, 11.90 mmol) and trifluoroacetic acid (2.2 mL, 30.0 mmol) at 0° C. After 1 h at 0° C., the reaction was quenched with saturated sodium bicarbonate and extracted with dichloromethane (2×25 mL). The organics were washed with brine, dried over magnesium sulfate, filtered, and concentrated to give a mixture of (3RS)-3-{3-chloro-4-[(triisopropylsilyl)oxy]phenyl}-1-oxa-8-azaspiro[4.5]decane and 3-{3-chloro-4-[(triisopropylsilyl)oxy]phenyl}-1-oxa-8-azaspiro[4.5]dec-3-ene. The crude oil was dissolved in methanol (25 mL) and Raney nickel (422 mg, 4.93 mmol) added and the solution stirred for 3 hours at 10 psi under a hydrogen atmosphere. The reaction was then filtered through celite and concentrated to give crude (3RS)-3-{3-chloro-4-[(triisopropylsilyl)oxy]phenyl}-1-oxa-8-azaspiro[4.5]decane. The crude product was dissolved in methanol (25 mL) to which was added di-tert butyl dicarbonate (1.0 g, 4.60 mmol) followed by triethylamine (0.49 mL, 3.54 mmol) and the reaction stirred at r.t. for 1 hour. The reaction was quenched with water and the aqueous phase extracted with ethyl acetate (2×15 mL), dried with magnesium sulfate and concentrated. The crude product was purified by flash chromatography (ethyl acetate:heptanes) to give the title compound as a clear oil (1.16 g, 2.21 mmol, 37%).

### tert-butyl 3-(3-chloro-4-hydroxyphenyl)-1-oxa-8-azaspiro[4.5]decane-8-carboxylate Enantiomers 1 and 2

tert-butyl (3RS)-3-{3-chloro-4-[(triisopropylsilyl)oxy]phenyl}-1-oxa-8-azaspiro[4.5]decane-8-carboxylate (2.3 g, 4.387 mmol) was dissolved in tetrahydrofuran (16 mL) and tetrabutylammonium fluoride (1.18 g, 4.39 mmol) added and the reaction stirred at r.t. for 1 hour. The reaction was then quenched with water and the aqueous phase extracted with ethyl acetate (2×25 mL), dried with magnesium sulfate and concentrated. The racemate was separated by chiral SFC on a 30×250 mm ChiralPak AD-H column (20% MeOH/CO₂ @ 70 mL/min; 2 mL injections of a 50 mg/mL MeOH solution). The first eluting peak gave Enantiomer 1 of the title compound as an oil (300 mg; t_{R}=1.51 min (4.6×100 mm Chiralpak AD-H, 20% MeOH/CO₂ at 3 mL/min)) and the second eluting peak gave Enantiomer 2 of the title compound as an oil (300 mg; t_{R}=3.98 min (4.6×100 mm Chiralpak AD-H, 20% MeOH/CO₂ at 3 ml/min)). m/z 268 (MH⁺ minus Boc).

### Example 43

### 3-{3-chloro-4-[(2-chlorobenzyl)oxy]phenyl}-N-(3,4-dimethylisoxazol-5-yl)-1-oxa-8-azaspiro[4.5]decane-8-carboxamide Enantiomer 1

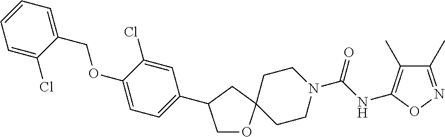

The title compound was prepared from tert-butyl 3-(3-chloro-4-hydroxyphenyl)-1-oxa-8-azaspiro[4.5]decane-8-carboxylate Enantiomer 1 (150 mg, 0.408 mmol), 2-chlorobenzyl bromide (168 mg, 0.816 mmol) sodium iodide (4.5 mg, 0.030 mmol), potassium carbonate (317 mg, 2.30 mmol), phenyl (3,4-dimethylisoxazol-5-yl)carbamate (94.8 mg, 0.408 mmol), triethylamine (0.05 mL, 0.408 mmol) as described for Example 45. The crude product was purified by flash chromatography (ethyl acetate (5% ethanol)/heptane) to give the title compound as a light brown solid. (20 mg, 0.068 mmol, 9%). ¹H NMR (400 MHz, DMSO-d₆) δ ppm 9.12 (1H, br. s.), 7.64 (1H, br. s.), 7.53 (1H, br. s.), 7.35-7.48 (3H, m), 7.21 (2H, br. s.), 5.24 (2H, s), 4.11 (1H, br. s.), 3.45-3.70 (5H, m), 3.36-3.45 (2H, m), 2.20-2.38 (1H, m), 2.12 (3H, d, J=2.7 Hz), 1.50-1.86 (7H, m). m/z 531.8 (MH⁺).

### Example 44

### 3-{3-chloro-4-[(2-chlorobenzyl)oxy]phenyl}-N-(3,4-dimethylisoxazol-5-yl)-1-oxa-8-azaspiro[4.5]decane-8-carboxamide Enantiomer 2

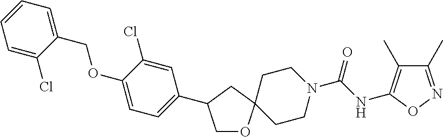

The title compound was prepared from tert-butyl 3-(3-chloro-4-hydroxyphenyl)-1-oxa-8-azaspiro[4.5]decane-8-carboxylate Enantiomer 2 (150 mg, 0.408 mmol), 2-chlorobenzyl bromide (168 mg, 0.816 mmol) sodium iodide (4.5 mg, 0.030 mmol), potassium carbonate (317 mg, 2.30 mmol), phenyl (3,4-dimethylisoxazol-5-yl)carbamate (94.8 mg, 0.408 mmol), triethylamine (0.05 mL, 0.408 mmol) as described for Example 45. The crude product was purified by flash chromatography (ethyl acetate (5% ethanol)/heptane) to give the title compound as a light brown solid. (100 mg, 0.188 mmol, 46%). ¹H NMR (400 MHz, DMSO-d₆) δ ppm 9.12 (1 H, br. s.), 7.64 (1H, br. s.), 7.53 (1H, br. s.), 7.35-7.48 (3H, m), 7.21 (2H, br. s.), 5.24 (2H, s), 4.11 (1H, br. s.), 3.45-3.70 (5H, m), 3.36-3.45 (2H, m), 2.20-2.38 (1H, m), 2.12 (3H, d, J=2.7 Hz), 1.50-1.86 (7 H, m). m/z 529.8 (MH⁺).

### Example 45

### 3-{3-chloro-4-[(3-chlorobenzyl)oxy]phenyl}-N-(3,4-dimethylisoxazol-5-yl)-1-oxa-8-azaspiro[4.5]decane-8-carboxamide Enantiomer 1

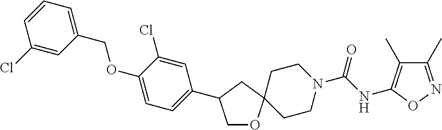

tert-butyl 3-(3-chloro-4-hydroxyphenyl)-1-oxa-8-azaspiro[4.5]decane-8-carboxylate Enantiomer 1 (150 mg, 0.408 mmol), 3-chlorobenzyl bromide (168 mg, 0.816 mmol) sodium iodide (4.5 mg, 0.030 mmol), potassium carbonate (317 mg, 2.30 mmol) were dissolved in acetone (1 mL) and the mixture heated in a sealed vial at 80° C. overnight. The reaction was filtered through a short plug of silica and concentrated. The crude product was dissolved in dichloromethane (1 mL) and trifluoromethanesulfonic acid (0.5 mL) added and the solution allowed to stir at r.t. for 1 hour. The reaction was concentrated and then redissoved in acetonitrile (1 mL). Phenyl (3,4-dimethylisoxazol-5-yl)carbamate (94.8 mg, 0.408 mmol) was then added followed by dropwise addition of triethylamine (0.05 mL, 0.408 mmol) and the reaction stirred for 30 minutes. The reaction was concentrated to give a light yellow oil. The crude product was purified by flash chromatography (ethyl acetate (5% ethanol):heptane) to give the title compound as a light brown solid. (50 mg, 0.094 mmol, 23%). ¹H NMR (400 MHz, DMSO-d₆) δ ppm 9.12 (1H, br. s.), 7.53 (1H, br. s.), 7.39-7.45 (3H, m), 7.20-7.27 (1H, m), 7.12-7.19 (1H, m), 5.22 (2H, br. s.), 4.10 (1H, t, J=7.7 Hz), 3.33-3.66 (7H, m), 2.22-2.32 (1H, m), 2.07-2.17 (3H, m), 1.51-1.82 (7H, m), 1.27 (1H, t, J=6.0 Hz). m/z 529.8 (MH⁺).

### Example 46

### 3-{3-chloro-4-[(3-chlorobenzyl)oxy]phenyl}-N-(3,4-dimethylisoxazol-5-yl)-1-oxa-8-azaspiro[4.5]decane-8-carboxamide Enantiomer 2

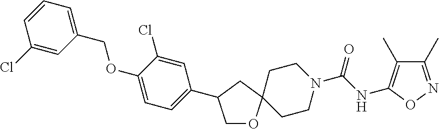

The title compound was prepared from tert-butyl 3-(3-chloro-4-hydroxyphenyl)-1-oxa-8-azaspiro[4.5]decane-8-carboxylate Enantiomer 2 (150 mg, 0.408 mmol), 3-chlorobenzyl bromide (168 mg, 0.816 mmol) sodium iodide (4.5 mg, 0.030 mmol), potassium carbonate (317 mg, 2.30 mmol), phenyl (3,4-dimethylisoxazol-5-yl)carbamate (94.8 mg, 0.408 mmol), triethylamine (0.05 mL, 0.408 mmol) as described for Example 45. The crude product was purified by flash chromatography (ethyl acetate (5% ethanol):heptane) to give the title compound as a light brown solid. (26 mg, 0.049 mmol, 12%). ¹H NMR (400 MHz, DMSO-d₆) δ ppm 9.12 (1H, br. s.), 7.53 (1H, br. s.), 7.39-7.45 (3H, m), 7.20-7.27 (1H, m), 7.12-7.19 (1H, m), 5.22 (2H, br. s.), 4.10 (1H, t, J=7.7 Hz), 3.33-3.66 (7H, m), 2.22-2.32 (1H, m), 2.07-2.17 (3H, m), 1.51-1.82 (7H, m), 1.27 (1H, t, J=6.0 Hz). m/z 529.8 (MH⁺).

### (3R)-3-{4-[(3-fluorobenzyl)oxy]phenyl}-1-oxa-8-azaspiro[4.5]decane hydrochloride

The title compound was prepared from tert-butyl (3R)-3-(4-hydroxyphenyl)-1-oxa-8-azaspiro[4.5]decane-8-carboxylate (300 mg, 0.90 mmol) and 3-fluorobenzyl bromide (0.203 mL, 1.66 mmol; CAS# 456-41-7) as described for (3R)-3-{4-[(4-fluorobenzyl)oxy]phenyl}-1-oxa-8-azaspiro[4.5]decane hydrochloride to give the title compound as a solid (316 mg, 84%). m/z 342 (MH⁺).

### Example 47

### (3R)—N-(3,4-dimethylisoxazol-5-yl)-3-{4-[(3-fluorobenzyl)oxy]phenyl}-1-oxa-8-azaspiro[4.5]decane-8-carboxamide

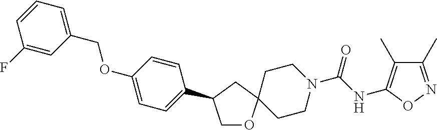

The title compound was prepared from (3R)-3-{4-[(3-fluorobenzyl)oxy]phenyl}-1-oxa-8-azaspiro[4.5]decane hydrochloride (200 mg, 0.529 mmol) as described for Example 41 without recrystallization from EA to give the title compound as a solid (210 mg, 83%). A sample (72 mg) was recrystallized from hot EA (˜4 mL) to give the title compound as a solid (53 mg, 74%). m/z 480 (MH⁺). ¹H NMR (400 MHz, DMSO-d₆) δ ppm 1.52-1.70 (m, 4H), 1.70-1.78 (m, 4H), 2.09-2.15 (m, 3H), 2.25 (dd, J=12.46, 7.68 Hz, 1H), 3.34-3.51 (m, 3 H), 3.51-3.63 (m, 3H), 4.08 (t, J=7.68 Hz, 1H), 5.11 (s, 2H), 6.95 (d, J=8.88 Hz, 2H), 7.11-7.18 (m, 1H), 7.18-7.30 (m, 4H), 7.38-7.48 (m, 1H), 9.11 (s, 1H).

### (3S)-3-{4-[(3-fluorobenzyl)oxy]phenyl}-1-oxa-8-azaspiro[4.5]decane hydrochloride

The title compound was prepared from tert-butyl (3S)-3-(4-hydroxyphenyl)-1-oxa-8-azaspiro[4.5]decane-8-carboxylate (300 mg, 0.90 mmol) and 3-fluorobenzyl bromide (0.203 mL, 1.66 mmol; CAS# 456-41-7) as described for (3R)-3-{4-[(4-fluorobenzyl)oxy]phenyl}-1-oxa-8-azaspiro[4.5]decane hydrochloride to give the title compound as a solid (282 mg, 75%). m/z 342 (MH⁺).

### Example 48

### (3S)—N-(3,4-dimethylisoxazol-5-yl)-3-{4-[(3-fluorobenzyl)oxy]phenyl}-1-oxa-8-azaspiro[4.5]decane-8-carboxamide

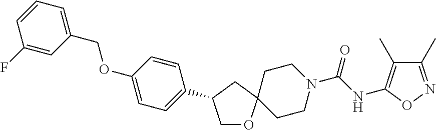

The title compound was prepared from (3S)-3-{4-[(3-fluorobenzyl)oxy]phenyl}-1-oxa-8-azaspiro[4.5]decane hydrochloride (200 mg, 0.529 mmol) as described for Example 41 without recrystallization from EA to give the title compound as a solid (188 mg, 74%). A sample (72 mg) was recrystallized from hot EA (˜4 mL) to give the title compound as a solid (52 mg, 72%). m/z 480 (MH⁺). ¹H NMR (400 MHz, DMSO-d₆) δ ppm 1.52-1.70 (m, 4H), 1.69-1.79 (m, 4H), 2.08-2.15 (m, 3H), 2.25 (dd, J=12.46, 8.02 Hz, 1H), 3.34-3.51 (m, 3 H), 3.51-3.64 (m, 3H), 4.09 (t, J=7.68 Hz, 1H), 5.11 (s, 2H), 6.95 (d, J=8.53 Hz, 2H), 7.15 (dt, J=8.70, 2.39 Hz, 1H), 7.19-7.31 (m, 4H), 7.38-7.48 (m, 1H), 9.11 (s, 1H).

### Example 49

### (3R)-3-{4-[(4-chlorobenzyl)oxy]phenyl}-N-(3,4-dimethylisoxazol-5-yl)-1-oxa-8-azaspiro[4.5]decane-8-carboxamide

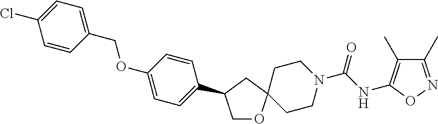

To tert-butyl (3R)-3-(4-hydroxyphenyl)-1-oxa-8-azaspiro[4.5]decane-8-carboxylate (300 mg, 0.90 mmol) was added 4-chlorobenzyl bromide (240 mg, 1.17 mmol), sodium iodide (10 mg, 0.07 mmol), potassium carbonate (498 mg, 3.60 mmol) and acetone (5 mL). The mixture was heated at 70° C. overnight. Another portion of 4-chlorobenzyl bromide (100 mg, 0.487 mmol) was added and the mixture was heated at 70° C. overnight. The mixture was treated with water (˜20 mL) and extracted with ethyl acetate (3×10 mL). The organics were evaporated to give the crude residue. The residue in DCM (2.5 mL) was treated with 4 N HCl/dioxane (2.5 mL). The solution was stirred for 3 hr at RT and then evaporated. The resulting residue was then treated with phenyl (3,4-dimethylisoxazol-5-yl)carbamate (230 mg, 0.990 mmol), acetonitrile (5 mL), DIEA (0.627 mL, 3.60 mmol) and stirred overnight. The resulting precipitate was collected by vacuum filtration and purified by flash chromatography (80-100% EA/Heptane) to give the title compound as a solid (285 mg, 58%). m/z 496 (MH⁺). ¹H NMR (400 MHz, DMSO-d₆) δ ppm 1.52-1.61 (m, 1H), 1.61-1.70 (m, 3H), 1.70-1.78 (m, 4H), 2.12 (s, 3H), 2.25 (dd, J=12.29, 7.85 Hz, 1H), 3.34-3.50 (m, 3H), 3.51-3.62 (m, 3H), 4.08 (t, J=7.68 Hz, 1 H), 5.08 (s, 2H), 6.90-6.98 (m, 2H), 7.21 (d, J=8.88 Hz, 2H), 7.41-7.49 (m, 4H), 9.11 (s, 1H). Assignment of absolute stereochemistry was confirmed by x-ray crystallography of crystals of the title compound obtained from acetonitrile.

### Example 50

### (3S)-3-{4-[(4-chlorobenzyl)oxy]phenyl}-N-(3,4-dimethylisoxazol-5-yl)-1-oxa-8-azaspiro[4.5]decane-8-carboxamide

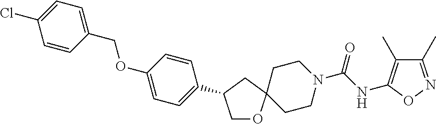

To tert-butyl (3S)-3-(4-hydroxyphenyl)-1-oxa-8-azaspiro[4.5]decane-8-carboxylate (300 mg, 0.90 mmol) was added 4-chlorobenzyl bromide (240 mg, 1.17 mmol), sodium iodide (10 mg, 0.07 mmol), potassium carbonate (498 mg, 3.60 mmol) and acetone (5 mL). The mixture was heated at 70° C. overnight. Another portion of 4-chlorobenzyl bromide (100 mg, 0.487 mmol) was added and the mixture was heated at 70° C. overnight. The mixture was treated with water (˜20 mL) and extracted with ethyl acetate (3×10 mL). The organics were evaporated to give the crude residue. The residue in DCM (2.5 mL) was treated with 4 N HCl/dioxane (2.5 mL). The solution was stirred for 3 hr at RT and then evaporated. The resulting residue was then treated with phenyl (3,4-dimethylisoxazol-5-yl)carbamate (230 mg, 0.990 mmol), acetonitrile (5 mL), DIEA (0.627 mL, 3.60 mmol) and stirred overnight. The resulting precipitate was collected by vacuum filtration, triturated with IPA and purified by flash chromatography (100% EA/Heptane) to give the title compound as a solid (173 mg, 35%). m/z 496 (MH⁺). ¹H NMR (400 MHz, DMSO-d₆) δ ppm 1.52-1.61 (m, 1H), 1.61-1.69 (m, 3H), 1.69-1.78 (m, 4H), 2.11 (s, 3H), 2.24 (dd, J=12.29, 7.85 Hz, 1H), 3.33-3.51 (m, 3H), 3.51-3.62 (m, 3H), 4.08 (t, J=7.68 Hz, 1H), 5.07 (s, 2H), 6.94 (d, J=8.88 Hz, 2H), 7.21 (d, J=8.53 Hz, 2H), 7.39-7.51 (m, 4H), 9.11 (s, 1H).

### (3R)-3-{4-[(3,4-difluorobenzyl)oxy]phenyl}-1-oxa-8-azaspiro[4.5]decane hydrochloride

The title compound was prepared from tert-butyl (3R)-3-(4-hydroxyphenyl)-1-oxa-8-azaspiro[4.5]decane-8-carboxylate (300 mg, 0.90 mmol) and 3,4-difluorobenzyl bromide (373 mg, 1.80 mmol; CAS# 85118-01-0) as described for (3R)-3-{4-[(4-fluorobenzyl)oxy]phenyl}-1-oxa-8-azaspiro[4.5]decane hydrochloride to give the title compound as a solid (329 mg, 84%). m/z 360 (MH⁺).

### Example 51

### (3R)-3-{4-[(3,4-difluorobenzyl)oxy]phenyl}-N-(3,4-dimethylisoxazol-5-yl)-1-oxa-8-azaspiro[4.5]decane-8-carboxamide

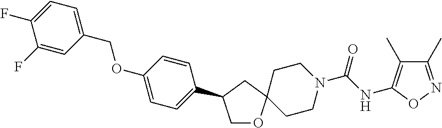

The title compound was prepared from (3R)-3-{4-[(3,4-difluorobenzyl)oxy]phenyl}-1-oxa-8-azaspiro[4.5]decane hydrochloride (200 mg, 0.529 mmol) as described for Example 41 (except that the product crystallized after evaporation of the chromatography fractions to a small volume (˜10 mL) and as such was not further recrystallized) to give the title compounds as a solid (189 mg, 75%). m/z 498 (MH⁺). ¹H NMR (400 MHz, DMSO-d₆) δ ppm 1.52-1.79 (m, 8H), 2.07-2.16 (m, 3H), 2.25 (dd, J=11.95, 8.19 Hz, 1H), 3.34-3.51 (m, 3H), 3.51-3.65 (m, 3H), 4.03-4.14 (m, 1H), 5.07 (s, 2H), 6.95 (d, J=8.53 Hz, 2H), 7.22 (d, J=8.53 Hz, 2H), 7.30 (br. s., 1H), 7.40-7.56 (m, 2H), 9.11 (s, 1H).

### (3S)-3-{4-[(3,4-difluorobenzyl)oxy]phenyl}-1-oxa-8-azaspiro[4.5]decane hydrochloride

The title compound was prepared from tert-butyl (3S)-3-(4-hydroxyphenyl)-1-oxa-8-azaspiro[4.5]decane-8-carboxylate (300 mg, 0.90 mmol) and 3,4-difluorobenzyl bromide (373 mg, 1.80 mmol; CAS# 85118-01-0) as described for (3R)-3-{4-[(4-fluorobenzyl)oxy]phenyl}-1-oxa-8-azaspiro[4.5]decane hydrochloride to give the title compound as a solid (304 mg, 78%). m/z 360 (MH⁺).

### Example 52

### (3S)-3-{4-[(3,4-difluorobenzyl)oxy]phenyl}-N-(3,4-dimethylisoxazol-5-yl)-1-oxa-8-azaspiro[4.5]decane-8-carboxamide

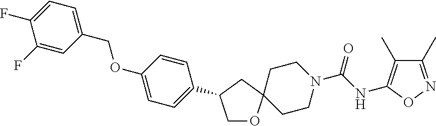

The title compound was prepared from (3S)-3-{4-[(3,4-difluorobenzyl)oxy]phenyl}-1-oxa-8-azaspiro[4.5]decane hydrochloride (200 mg, 0.529 mmol) as described for Example 41 to give the title compound as a solid (152 mg, 61%). m/z 498 (MH⁺). ¹H NMR (400 MHz, DMSO-d₆) δ ppm 1.51-1.79 (m, 8 H), 2.12 (s, 3H), 2.25 (dd, J=12.12, 8.36 Hz, 1H), 3.34-3.52 (m, 3H), 3.51-3.65 (m, 3H), 4.02-4.13 (m, 1 H), 5.07 (s, 2H), 6.95 (d, J=8.53 Hz, 2H), 7.22 (d, J=8.53 Hz, 2H), 7.30 (br. s., 1H), 7.39-7.57 (m, 2H), 9.11 (s, 1H).

### tert-butyl (3RS)-3-(3-{[(trifluoromethyl)sulfonyl]oxy}phenyl)-1-oxa-8-azaspiro[4.5]decane-8-carboxylate

In a 500 mL flask was added tert-butyl (3RS)-3-(3-hydroxyphenyl)-1-oxa-8-azaspiro[4.5]decane-8-carboxylate (3.2 g, 9.6 mmol), pyridine (2.24 mL, 28.8 mmol) in 100 mL DCM. The mixture was cooled with an ice-bath while trifluoromethanesulfonic anhydride (2.43 mL, 14.4 mmol) was added dropwise. The mixture was stirred at room temperature for 1 hour. The mixture was washed with sodium bicarbonate, brine, and dried over sodium sulfate. The crude was purified by column chromatography (20% EtOAc in hexane) to give the title compound (4.3 g, 96%) as a colorless oil. ¹H NMR (DMSO-d₆) δ ppm 7.52 (m, 1H), 7.47 (m, 2H), 7.34 (m, 1H), 4.12 (m, 1H), 3.60-3.70 (m, 2H), 3.40-3.50 (m, 2H), 3.20-3.30 (m, 2H), 2.26 (m, 1H), 1.78 (m, 1H), 1.50-1.70 (m, 4H), 1.40 (s, 9H). LCMS found M+H=466.

### Examples 53-56

A 0.1 M solution of tert-butyl (3RS)-3-(3-{[(trifluoromethyl)sulfonyl]oxy}phenyl)-1-oxa-8-azaspiro[4.5]decane-8-carboxylate in DMF (1 mL), a 0.025 M solution of Pd(PPh₃)₄ in DMF (0.2 mL), and a 1 M aqueous solution of sodium carbonate (0.3 mL) were added to the corresponding aryl boronic acids (0.125 mmol) in vials (Note: the solutions were purged with nitrogen prior to addition). The vials were capped and heated to 100° C. for 18 h. The solvents were evaporated. The residues were partitioned between dichloromethane (2 mL) and water (1 mL). The organic layers were treated with trifluoroacetic acid (0.5 mL). After 1 h at room temp, the solvents were evaporated. A 0.1 M solution of phenyl (3,4-dimethylisoxazol-5-yl)carbamate in acetonitrile (1.0 mL) and triethylamine (0.060 mmol) was added to the residues. The vials were capped and shook for 4 h at room temp. The solvents were evaporated and the residues were dissolved in DMSO (1.5 mL) and purified by reverse phase HPLC (acetonitrile/water/0.05% trifluoroacetic acid) to give racemic Examples 53-56. The purified compounds were analyzed by LCMS (Phenomenex Gemini C18 4.6×50 mm 5 μm; 10 mM ammonium bicarbonate pH 8.2/MeCN).

| Ex. | Name | t_{R} (min) | m/z (MH⁺) |
|---|---|---|---|
| 53 | (3RS)-3-(3′,4′-difluorobiphenyl-3-yl)-N- | 2.11 | 468.15 |
| | (3,4-dimethylisoxazol-5-yl)-1-oxa-8- | | |
| | azaspiro[4.5]decane-8-carboxamide | | |
| 54 | (3RS)-N-(3,4-dimethylisoxazol-5-yl)-3-(4′- | 2.05 | 450.35 |
| | fluorobiphenyl-3-yl)-1-oxa-8-azaspiro[4.5] | | |
| | decane-8-carboxamide | | |
| 55 | (3RS)-3-(4′-chlorobiphenyl-3-yl)-N-(3,4- | 2.24 | 466.45 |
| | dimethylisoxazol-5-yl)-1-oxa-8-azaspiro[4.5] | | |
| | decane-8-carboxamide | | |
| 56 | (3RS)-N-(3,4-dimethylisoxazol-5-yl)-3-[4′- | 2.31 | 516.15 |
| | (trifluoromethoxy)biphenyl-3-yl]-1-oxa-8- | | |
| | azaspiro[4.5]decane-8-carboxamide | | |

### tert-butyl (3S)-3-(3-{[(trifluoromethyl)sulfonyl]oxy}phenyl)-1-oxa-8-azaspiro[4.5]decane-8-carboxylate

To a solution of tert-butyl (3S)-3-(3-hydroxyphenyl)-1-oxa-8-azaspiro[4.5]decane-8-carboxylate (1.1 g, 3.3 mmol) and pyridine (0.817 mL, 9.9 mmol) in DCM (30 mL) at 0° C. was added trifluoromethanesulfonic anhydride (0.853 mL, 4.95 mmol). The solution was allowed to warm to RT and stirred for 20 min. The organic was washed with sat. sodium bicarbonate (˜100 mL) and the aqueous was extracted with DCM (2×50 mL). The organic was dried over magnesium sulfate and filtered evaporated. The resulting oil was purified by flash chromatography (5-40% EA/Heptane) to give the title compound as an oil (1.46 g, 95%). m/z 410 (MH⁺ minus tBu).

### Example 57

### (3S)-3-(3′,4′-difluorobiphenyl-3-yl)-N-(3,4-dimethylisoxazol-5-yl)-1-oxa-8-azaspiro[4.5]decane-8-carboxamide

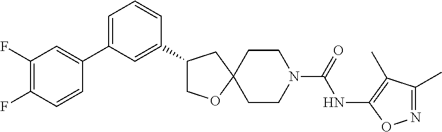

The title compound was prepared from tert-butyl (3S)-3-(3-{[(trifluoromethyl)sulfonyl]oxy}phenyl)-1-oxa-8-azaspiro[4.5]decane-8-carboxylate (365 mg, 0.784 mmol) and (3,4-difluorophenyl)boronic acid (186 mg, 1.18 mmol) as described for Example 59 except that the chromatography product was evaporated to give an oil. The oil was purified by reverse phase chromatography (10-95% acetonitrile/water/0.05% TFA) and evaporated to a small volume to give an aqueous mixture. The mixture was treated with EA (50 mL) and the layers separated. The organic layer was washed with 0.25 M NaOH (2×50 mL), brine (50 mL), dried over magnesium sulfate, filtered and evaporated to give a solid upon standing (266 mg, 73%). m/z 468 (MH⁺). ¹H NMR (400 MHz, DMSO-d₆) δ ppm 1.55-1.77 (m, 7H), 1.88 (dd, J=12.3, 10.9 Hz, 1H), 2.12 (s, 3H), 2.33 (dd, J=12.3, 7.9 Hz, 1H), 3.36-3.48 (m, 2H), 3.52-3.66 (m, 3H), 3.70-3.78 (m, 1H), 4.17 (t, J=7.7 Hz, 1 H), 7.30-7.36 (m, 1H), 7.41 (t, J=7.7 Hz, 1H), 7.47-7.57 (m, 3H), 7.60 (s, 1H), 7.75-7.85 (m, 1H), 9.13 (br. s., 1H).

### tert-butyl (3R)-3-(3-{[(trifluoromethyl)sulfonyl]oxy}phenyl)-1-oxa-8-azaspiro[4.5]decane-8-carboxylate

To a solution of tert-butyl (3R)-3-(3-hydroxyphenyl)-1-oxa-8-azaspiro[4.5]decane-8-carboxylate (2.00 g, 6.00 mmol) and pyridine (1.48 mL, 18.0 mmol) in DCM (60 mL) at 0° C. was added trifluoromethanesulfonic anhydride (1.55 mL, 9.00 mmol). The solution was allowed to warm to RT and stirred for 20 min. The organic was washed with sat. sodium bicarbonate (˜100 mL) and the aqueous was extracted with DCM (2×50 mL). The organic was dried over magnesium sulfate and filtered evaporated. The resulting oil was purified by flash chromatography (5-40% EA/Heptane) to give the title compound as an oil (2.79 g, 95%). m/z 410 (MH⁺ minus tBu). ¹H NMR (400 MHz, CDCl₃) δ ppm 1.47 (s, 9H), 1.59 (dd, J=10.07, 3.93 Hz, 1H), 1.67-1.82 (m, 4H), 2.30 (dd, J=12.63, 8.19 Hz, 1H), 3.29-3.41 (m, 2H), 3.50-3.60 (m, 1H), 3.64 (br. s., 2H), 3.80 (t, J=8.53 Hz, 1H), 4.20-4.27 (m, 1H), 7.12-7.17 (m, 2H), 7.26-7.30 (m, 1H), 7.37-7.43 (m, 1H).

### Example 58

### (3R)-3-(3′,4′-difluorobiphenyl-3-yl)-N-(3,4-dimethylisoxazol-5-yl)-1-oxa-8-azaspiro[4.5]decane-8-carboxamide

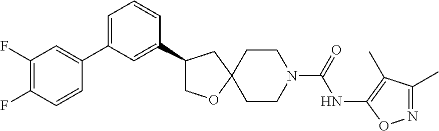

The title compound was prepared from tert-butyl (3R)-3-(3-{[(trifluoromethyl)sulfonyl]oxy}phenyl)-1-oxa-8-azaspiro[4.5]decane-8-carboxylate (300 mg, 0.633 mmol) and (3,4-difluorophenyl)boronic acid (150 mg, 0.95 mmol) as described for Example 59 except that the chromatography product was evaporated to give a solid. The solid was purified by reverse phase chromatography (10-95% acetonitrile/water/0.05% TFA) and evaporated to a small volume to give an aqueous mixture. The mixture was treated with EA (20 mL) and the layers separated. The organic layer was washed with 0.1 M NaOH (20 mL), brine, dried over magnesium sulfate, filtered and evaporated to give an oil. The oil was dissolved in EtOH (˜4 mL) and then evaporated to give the title compound as an oil (225 mg, 70%). m/z 468 (MH⁺). ¹H NMR (400 MHz, DMSO-d₆) δ ppm 1.55-1.73 (m, 4H), 1.74 (s, 3H), 1.88 (dd, J=12.3, 10.9 Hz, 1H), 2.12 (s, 3H), 2.33 (dd, J=12.3, 7.9 Hz, 1H), 3.35-3.47 (m, 2H), 3.52-3.65 (m, 3H), 3.70-3.78 (m, 1H), 4.17 (t, J=7.9 Hz, 1H), 7.30-7.36 (m, 1H), 7.41 (t, J=7.7 Hz, 1H), 7.47-7.57 (m, 3H), 7.60 (s, 1H), 7.75-7.84 (m, 1H), 9.13 (br. s., 1H).

### Example 59

### (3R)—N-(3,4-dimethylisoxazol-5-yl)-3-(4′-fluorobiphenyl-3-yl)-1-oxa-8-azaspiro[4.5]decane-8-carboxamide

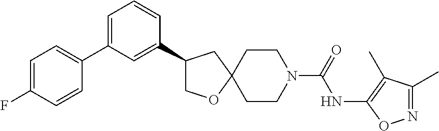

To a stirring solution of tert-butyl (3R)-3-(3-{[(trifluoromethyl)sulfonyl]oxy}phenyl)-1-oxa-8-azaspiro[4.5]decane-8-carboxylate (300 mg, 0.633 mmol) and (4-fluorophenyl)boronic acid (133 mg, 0.95 mmol) in toluene (4 mL) and isopropy alcohol (4 mL) was added a solution of cesium carbonate (1.03 g, 3.16 mmol) in water (0.63 mL). The mixture was stirred for 15 min at rt. at which time Pd(dppf)₂Cl₂ (0.062 g, 0.085 mmol; CAS # 72287-26-4) was added, the reaction was blanketed with argon and the vial was capped. The mixture was heated at 110° C. with stirring overnight. The resulting mixture was treated with magnesium sulphate, 3-mercaptopropyl-functionalized silicagel (aldrich from silicycle) and was stirred overnight. The mixture was filtered and evaporated. To the crude residue in DCM (2.5 mL) was added 4 N HCl/dioxane (2.5 mL). The solution was stirred for 3 hr at RT and then evaporated. The resulting residue was treated with phenyl (3,4-dimethylisoxazol-5-yl)carbamate (162 mg, 0.696 mmol), acetonitrile (4 mL), DIEA (0.441 mL, 2.53 mmol) and stirred overnight. The reaction was filtered through a PTFE hydrophilic membrane (0.45 um) with ethyl acetate and evaporated. The crude material was purified by flash chromatography (50-100% EA/Heptane). The desired fractions were evaporated to a small volume and crystals formed upon standing. The crystals were dried under vacuum to give the title compound as a solid (229 mg, 80%). m/z 450 (MH+). ¹H NMR (400 MHz, DMSO-d₆) δ ppm 1.55-1.73 (m, 4H), 1.74 (s, 3H), 1.82-1.92 (m, 1H), 2.12 (s, 3H), 2.34 (dd, J=12.29, 7.85 Hz, 1H), 3.36-3.47 (m, 2H), 3.52-3.66 (m, 3H), 3.74 (t, J=8.88 Hz, 1H), 4.18 (t, J=7.68 Hz, 1H), 7.24-7.34 (m, 3H), 7.40 (t, J=7.51 Hz, 1H), 7.49 (d, J=7.17 Hz, 1H), 7.55 (s, 1H), 7.66-7.76 (m, 2H), 9.12 (s, 1H).

### Example 60

### (3S)—N-(3,4-dimethylisoxazol-5-yl)-3-(4′-fluorobiphenyl-3-yl)-1-oxa-8-azaspiro[4.5]decane-8-carboxamide

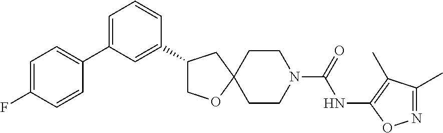

The title compound was prepared from tert-butyl (3S)-3-(3-{[(trifluoromethyl)sulfonyl]oxy}phenyl)-1-oxa-8-azaspiro[4.5]decane-8-carboxylate (365 mg, 0.784 mmol) and (4-fluorophenyl)boronic acid (164 mg, 1.18 mmol) as described for Example 59 to give the title compound as a solid (217 mg, 61%). m/z 450 (MH⁺). ¹H NMR (400 MHz, DMSO-d₆) δ ppm 1.55-1.73 (m, 4H), 1.74 (s, 3H), 1.87 (t, J=11.44 Hz, 1H), 2.12 (s, 3H), 2.34 (dd, J=12.29, 7.85 Hz, 1H), 3.35-3.48 (m, 2H), 3.52-3.66 (m, 3H), 3.74 (t, J=8.88 Hz, 1H), 4.18 (t, J=7.68 Hz, 1H), 7.29 (t, J=8.70 Hz, 3H), 7.40 (t, J=7.68 Hz, 1H), 7.49 (d, J=7.85 Hz, 1H), 7.55 (s, 1H), 7.67-7.76 (m, 2H), 9.12 (s, 1H).

### Example 61

### (3R)-3-(4′-chlorobiphenyl-3-yl)-N-(3,4-dimethylisoxazol-5-yl)-1-oxa-8-azaspiro[4.5]decane-8-carboxamide

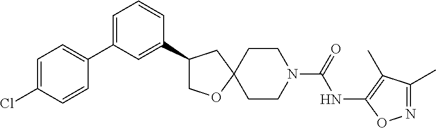

The title compound was prepared from tert-butyl (3R)-3-(3-{[(trifluoromethyl)sulfonyl]oxy}phenyl)-1-oxa-8-azaspiro[4.5]decane-8-carboxylate (300 mg, 0.633 mmol) and (4-chlorophenyl)boronic acid (150 mg, 0.95 mmol) as described for Example 59 except the chromatography product was evaporated to give a solid. Further purification by reverse phase chromatography (10-95% acetonitrile/water/0.05% TFA) and evaporation to a small volume gave an aqueous mixture. The mixture was treated with EA (100 mL) and the layers separated. The organic layer was washed with 0.1 M NaOH (50 mL), brine, dried over magnesium sulfate, filtered and evaporated to give a solid. The solid was recrystallized from EtOH to give the title compound as a solid (118 mg, 40%). m/z 466 (MH⁺). ¹H NMR (400 MHz, DMSO-d₆) δ ppm 1.56-1.76 (m, 7 H), 1.87 (dd, J=12.3, 10.6 Hz, 1H), 2.12 (s, 3H), 2.34 (dd, J=12.3, 7.9 Hz, 1H), 3.35-3.48 (m, 2H), 3.53-3.65 (m, 3H), 3.70-3.77 (m, 1H), 4.18 (t, J=7.7 Hz, 1H), 7.33 (d, J=7.9 Hz, 1H), 7.42 (t, J=7.5 Hz, 1H), 7.48-7.54 (m, 3H), 7.58 (s, 1H), 7.67-7.73 (m, 2H), 9.13 (br. s., 1H).

### Example 62

### (3S)-3-(4′-chlorobiphenyl-3-yl)-N-(3,4-dimethylisoxazol-5-yl)-1-oxa-8-azaspiro[4.5]decane-8-carboxamide

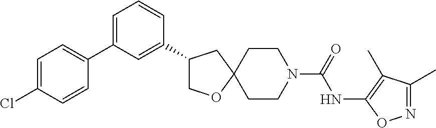

The title compound was prepared from tert-butyl (3S)-3-(3-{[(trifluoromethyl)sulfonyl]oxy}phenyl)-1-oxa-8-azaspiro[4.5]decane-8-carboxylate (365 mg, 0.784 mmol) and (4-chlorophenyl)boronic acid (184 mg, 1.18 mmol) as described for Example 59 except that the chromatography product was evaporated to give a solid. The solid was recrystallized from EtOH to give the title compound as a solid (57 mg, 16%). m/z 466 (MH⁺). ¹H NMR (400 MHz, DMSO-d₆) δ ppm 1.55-1.76 (m, 7H), 1.83-1.92 (m, 1H), 2.12 (s, 3H), 2.34 (dd, J=12.3, 7.9 Hz, 1H), 3.35-3.47 (m, 2H), 3.50-3.65 (m, 3H), 3.74 (t, J=8.9 Hz, 1H), 4.17 (t, J=7.7 Hz, 1H), 7.33 (d, J=7.5 Hz, 1H), 7.42 (t, J=7.5 Hz, 1H), 7.52 (d, J=8.5 Hz, 3H), 7.58 (s, 1H), 7.70 (d, J=8.2 Hz, 2H), 9.13 (br. s., 1H).

### Example 63

### (3S)—N-(3,4-dimethylisoxazol-5-yl)-3-[4′-(trifluoromethoxy)biphenyl-3-yl]-1-oxa-8-azaspiro[4.5]decane-8-carboxamide

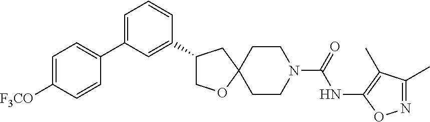

The title compound was prepared from tert-butyl (3S)-3-(3-{[(trifluoromethyl)sulfonyl]oxy}phenyl)-1-oxa-8-azaspiro[4.5]decane-8-carboxylate (365 mg, 0.784 mmol) and (4-trifluoromethoxyphenyl)boronic acid (242 mg, 1.18 mmol) as described for Example 57 to give the title compound as a solid upon standing (335 mg, 78%). m/z 516 (MH⁺). ¹H NMR (400 MHz, DMSO-d₆) δ ppm 1.55-1.77 (m, 7H), 1.83-1.93 (m, 1H), 2.12 (s, 3H), 2.34 (dd, J=12.6, 7.9 Hz, 1H), 3.36-3.47 (m, 2H), 3.52-3.67 (m, 3H), 3.70-3.79 (m, 1H), 4.18 (t, J=7.9 Hz, 1H), 7.34 (d, J=7.5 Hz, 1H), 7.40-7.49 (m, 3H), 7.53 (d, J=7.5 Hz, 1H), 7.59 (s, 1H), 7.76-7.82 (m, 2H), 9.13 (br. s., 1H).

### Example 64

### (3R)—N-(3,4-dimethylisoxazol-5-yl)-3-[4′-(trifluoromethoxy)biphenyl-3-yl]-1-oxa-8-azaspiro[4.5]decane-8-carboxamide

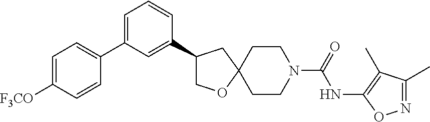

The title compound was prepared from tert-butyl (3R)-3-(3-{[(trifluoromethyl)sulfonyl]oxy}phenyl)-1-oxa-8-azaspiro[4.5]decane-8-carboxylate (300 mg, 0.633 mmol) and (4-trifluoromethoxyphenyl)boronic acid (196 mg, 0.95 mmol) as described for Example 58 to give the title compound as a solid upon standing (240 mg, 74%). m/z 516 (MH⁺). ¹H NMR (400 MHz, DMSO-d₆) δ ppm 1.55-1.72 (m, 4H), 1.73 (s, 3H), 1.83-1.92 (m, 1H), 2.11 (s, 3H), 2.34 (dd, J=12.5, 8.0 Hz, 1H), 3.34-3.47 (m, 2H), 3.57 (d, J=8.5 Hz, 3H), 3.70-3.78 (m, 1H), 4.18 (t, J=7.9 Hz, 1H), 7.31-7.36 (m, 1H), 7.39-7.48 (m, 3H), 7.52 (d, J=7.9 Hz, 1H), 7.59 (s, 1 H), 7.76-7.82 (m, 2H), 9.12 (br. s., 1H).

### tert-butyl 3-{[(trifluoromethyl)sulfonyl]oxy}-1-oxa-8-azaspiro[4.5]dec-3-ene-8-carboxylate

A solution of tert-butyl 3-oxo-1-oxa-8-azaspiro[4.5]decane-8-carboxylate (5.0 g, 20 mmol) in anhydrous THF (50 mL) was cooled to −78° C. and then treated with LiHMDS (1.0 M in THF, 27.4 mL, 27.4 mmol). The solution was stirred for 1 h at −78° C. then treated with a solution of the 2-[N,N-bis(trifluoromethanesulphonyl)amino]-5-chloropyridine (9.2 g, 23.5 mmol; CAS# 145100-51-2) in 5 ml of THF. The resulting solution was stirred at −78° C. for 1 h then warmed to room temperature for 30 min. The solution was quenched with water and extracted with ether. Ether layer was washed with water 3 times (50 mL). After drying in MgSO₄, the filtrate was concentrated to dryness and provided a brownish solid (6.72 g). The crude was purified by flash chromatography (10-20% EtOAc/heptane) to give the title compound as a white solid (5.9 g, 80%). ¹H NMR (400 MHz, CDCl₃) δ ppm 1.46 (s, 9H) 1.66-1.75 (m, 4H) 3.13-3.34 (m, 2 H) 3.75 (br. s., 2H) 4.64 (d, J=2.15 Hz, 2H) 5.75 (t, J=2.05 Hz, 1H). m/z 410.1 (M+Na).

### tert-butyl 3-[3-(trifluoromethyl)phenyl]-1-oxa-8-azaspiro[4.5]dec-3-ene-8-carboxylate

The tert-butyl 3-{[(trifluoromethyl)sulfonyl]oxy}-1-oxa-8-azaspiro[4.5]dec-3-ene-8-carboxylate (2.0 g, 5.16 mmo) was dissolved in toluene (16 mL). Then the mixture of (3-trifluoromethylphenyl)boronic acid (1.18 g, 6.2 mmol) in EtOH (4 mL) and 2M aqueous Na₂CO₃(7.74 mL, 15.5 mmol.) were added to the vial, and the resulting mixture was deoxygenated with a stream of Argon. After 10 min, Pd(PPh₃)₄ was added and the mixture was brought to reflux for 6 hours. The mixture was poured into water and extracted with ethyl acetate. The combined ethyl acetate layers were dried over MgSO₄. After filtration, the concentrated crude product was purified by silica gel chromatography with 10-20% EtOAc in heptane to provide the title compound (2 g, quant.). m/z 328 (MH⁺ minus t-Bu).

### tert-butyl (3RS)-3-[3-(trifluoromethyl)phenyl]-1-oxa-8-azaspiro[4.5]decane-8-carboxylate

The tert-butyl 3-{[(trifluoromethyl)sulfonyl]oxy}-1-oxa-8-azaspiro[4.5]dec-3-ene-8-carboxylate (2 g, 5.2 mmol) was dissolved in MeOH (30 mL), and Raney Nickel in water was added (2 spatula scoops). The reaction was run for 4 hours at 20 psi under hydrogen. The catalyst was filtered off through a silica plug and concentrated to provide the title compound as a racemate (2.0 g, quant.). m/z 330 (MH⁺ minus t-Bu).

### (3RS)-3-[3-(trifluoromethyl)phenyl]-1-oxa-8-azaspiro[4.5]decane hydrochloride

0.908 mL of 4M HCl in dioxane was added to the solution of tert-butyl (3RS)-3-[3-(trifluoromethyl)phenyl]-1-oxa-8-azaspiro[4.5]decane-8-carboxylate (350 mg, 0.908 mmol) in dichloromethane (10 mL). The mixture was stirred at room temperature overnight. The solvent and excess HCl was evaporated to provide the racemic title compound as a solid (292 mg, quant.). m/z 286.2 (MH⁺).

### Example 65

### (3RS)—N-(3,4-dimethylisoxazol-5-yl)-3-[3-(trifluoromethyl)phenyl]-1-oxa-8-azaspiro[4.5]decane-8-carboxamide

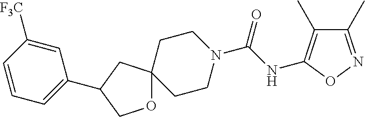

The title compound was prepared from (3RS)-3-[3-(trifluoromethyl)phenyl]-1-oxa-8-azaspiro[4.5]decane hydrochloride (32 mg, 0.099 mmol) using same procedure as described for preparation of Example 66 to provide the racemic title compound. ¹H NMR (400 MHz, DMSO-d₆) δ ppm 1.60 (dd, J=9.15, 4.03 Hz, 1H) 1.63-1.73 (m, 6H) 1.81 (dd, J=12.26, 10.43 Hz, 1H) 2.06-2.14 (m, 3H) 2.33 (dd, J=12.63, 7.50 Hz, 1H) 3.35-3.45 (m, 2H) 3.52-3.60 (m, 2H) 3.62-3.69 (m, 2H) 4.11-4.20 (m, 1H) 7.53-7.60 (m, 2H) 7.61-7.65 (m, 2H) 9.11 (br. s., 1H). m/z (MH⁺) 424.2.

### tert-butyl 3-[3-(trifluoromethyl)phenyl]-1-oxa-8-azaspiro[4.5]decane-8-carboxylate Enantiomers 1 and 2

tert-Butyl (3RS)-3-[3-(trifluoromethyl)phenyl]-1-oxa-8-azaspiro[4.5]decane-8-carboxylate (2.0 g) was separated by chiral SFC on a 30×250 mm ChiralPak AD-H column (10% MeOH/CO₂ @ 70 mL/min; 2 g sample in 40 mL methanol @ 50 mg/mL, 1 mL injections). The first eluting peak gave Enantiomer 1 of the title compound (700 mg, 70%; t_{R}=0.56 min (4.6×100 mm Chiralpak AD-H, 20% MeOH/CO₂ at 3 ml/min)). The second eluting peak gave Enantiomer 2 of the title compound (1000 mg, quant.; t_{R}=0.73 min (4.6×100 mm Chiralpak AD-H, 20% MeOH/CO₂ at 3 ml/min)). ¹H NMR (400 MHz, CDCl₃) δ ppm 1.44-1.54 (m, 9H) 1.54-1.64 (m, 2H) 1.69-1.78 (m, 1H) 1.82 (dd, J=12.50, 10.16 Hz, 1H) 2.30 (dd, J=12.50, 8.20 Hz, 1H) 3.28-3.47 (m, 2H) 3.49 (d, J=4.88 Hz, 1H) 3.52-3.61 (m, 1H) 3.62 (br. s., 2H) 3.81 (t, J=8.79 Hz, 1H) 4.20-4.31 (m, 1H) 7.40-7.54 (m, 4H). m/z 286.2 (MH⁺-Boc).

### 3-[3-(trifluoromethyl)phenyl]-1-oxa-8-azaspiro[4.5]decane hydrochloride Enantiomer 1

1.82 mL of 4M HCl in dioxane was added to the solution of tert-butyl 3-[3-(trifluoromethyl)phenyl]-1-oxa-8-azaspiro[4.5]decane-8-carboxylate Enantiomer 1 (700 mg, 1.82 mmol) in dichloromethane (20 mL). The mixture was stirred in room temperature overnight. The solvent and excess HCl was evaporated to provide the title compound as a white solid (560 mg, 95.8%). ¹H NMR (400 MHz, methanol-d₄) δ ppm 1.81-2.15 (m, 4H) 2.02 (dd, J=12.60, 7.91 Hz, 1H) 2.43 (dd, J=12.60, 7.91 Hz, 1H) 3.20-3.40 (m, 4H) 3.63-3.80 (m, 1 H) 3.85 (t, J=8.79 Hz, 1H) 4.29 (t, J=7.82 Hz, 1H) 7.46-7.71 (m, 4H). m/z 286.2 (MH⁺).

### Example 66

### N-(3,4-dimethylisoxazol-5-yl)-3-[3-(trifluoromethyl)phenyl]-1-oxa-8-azaspiro[4.5]decane-8-carboxamide Enantiomer 1

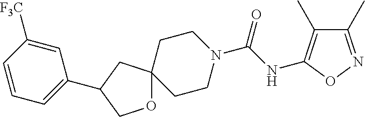

3-[3-(Trifluoromethyl)phenyl]-1-oxa-8-azaspiro[4.5]decane hydrochloride Enantiomer 1 (200 mg, 0.62 mmol) was dissolved in acetonitrile (10 mL) in a vial. Phenyl(3,4-dimethylisoxazol-5-yl)carbamate (144 mg, 0.622 mmol) was added, followed by addition of DIEA (161 mg, 1.24 mmol). The mixture was stirred at RT overnight. The mixture was concentrated to provide crude product which was purified by silica gel chromatography (20 to 40% ethyl acetate in heptane) to provide title product as white solid (195 mg, 74%). ¹H NMR (400 MHz, DMSO-d₆) δ ppm 1.60 (dd, J=8.97, 3.84 Hz, 1H) 1.63-1.73 (m, 6H) 1.81 (dd, J=12.26, 10.07 Hz, 1H) 2.07-2.14 (m, 3H) 2.33 (dd, J=12.63, 7.50 Hz, 1H) 3.36-3.42 (m, 2H) 3.52-3.60 (m, 2H) 3.62-3.69 (m, 2H) 4.12-4.19 (m, 1H) 7.53-7.65 (m, 4H) 9.11 (s, 1H). m/z (MH⁺) calc 424.1848, found 424.2091.

### 3-[3-(trifluoromethyl)phenyl]-1-oxa-8-azaspiro[4.5]decane hydrochloride Enantiomer 2

The title compound was prepared from tert-butyl 3-[3-(trifluoromethyl)phenyl]-1-oxa-8-azaspiro[4.5]decane-8-carboxylate Enantiomer 2 using same procedure as described for preparing for 3-[3-(trifluoromethyl)phenyl]-1-oxa-8-azaspiro[4.5]decane hydrochloride Enantiomer 1. ¹H NMR (400 MHz, methanol-d₄) δ ppm 1.81-2.15 (m, 4H) 2.43 (dd, J=12.60, 7.91 Hz, 1H) 3.20-3.40 (m, 4H) 3.63-3.80 (m, 1H) 3.85 (t, J=8.79 Hz, 1H) 4.29 (t, J=7.82 Hz, 1H) 4.87 (s, 3H) 7.46-7.71 (m, 4H). m/z 286.2 (MH⁺).

### Example 67

### N-(3,4-dimethylisoxazol-5-yl)-3-[3-(trifluoromethyl)phenyl]-1-oxa-8-azaspiro[4.5]decane-8-carboxamide Enantiomer 2

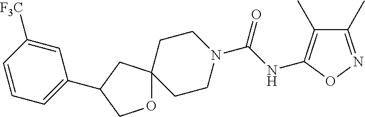

The title compound was prepared from 3-[3-(trifluoromethyl)phenyl]-1-oxa-8-azaspiro[4.5]decane hydrochloride Enantiomer 2 (200 mg, 0.62 mmol) as described for Example 66 to provide title compound (210 mg, 79.7%). ¹H NMR (400 MHz, DMSO-d₆) δ ppm 1.60 (dd, J=9.15, 4.03 Hz, 1H) 1.63-1.74 (m, 6H) 1.81 (dd, J=12.08, 10.25 Hz, 1H) 2.08-2.14 (m, 3H) 2.33 (dd, J=12.45, 7.32 Hz, 1H) 3.36-3.45 (m, 2H) 3.52-3.60 (m, 2H) 3.62-3.69 (m, 2H) 4.16 (t, J=6.95 Hz, 1H) 7.53-7.60 (m, 2H) 7.61-7.65 (m, 2H) 9.11 (s, 1 H). m/z (MH⁺) calc 424.1848, found 424.2087.

### Phenyl (3-ethyl-4-methylisoxazol-5-yl)carbamate

To a solution of 5-amino-3-ethyl-4-methylisoxazole (5.63 g, 44.6 mmol, 1.0 equiv; CAS# 153458-34-5) in acetonitrile (25 mL) at 0° C. was added triethylamine (6.53 mL, 46.8 mmol, 1.05 equiv) followed by phenyl chloroformate (5.91 mL, 46.8 mmol, 1.05 equiv) in 100 mL THF. After stirring at 0° C. for 1 h, the reaction was warmed to RT overnight. The reaction was diluted with ethyl acetate and washed with 2M HCl, water, saturated sodium bicarbonate, and brine. The organic layer was dried over magnesium sulfate, filtered, concentrated, and purified by flash chromatography (ethyl acetate/hexane) to give title compound as a white solid (4.0 g). ¹H NMR (400 MHz, DMSO-d₆) δ ppm 10.71 (br. s., 1H), 7.41-7.45 (m, 2H), 7.26-7.30 (m, 1 H), 7.22-7.26 (m, 2H), 2.58 (q, J=7.51 Hz, 2H), 1.87 (s, 3H), 1.17 (t, J=7.51 Hz, 3H). m/z 279.2 (MNa⁺).

### Example 68

### 3-(3,4-dichlorophenyl)-N-(3-ethyl-4-methylisoxazol-5-yl)-1-oxa-8-azaspiro[4.5]decane-8-carboxamide Enantiomer 1

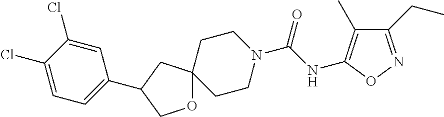

3-(3,4-Dichlorophenyl)-1-oxa-8-azaspiro[4.5]decane hydrochloride Enantiomer 1 (201 mg, 0.62 mmol) was dissolved in acetonitrile (10 mL) in a vial, phenyl (3-ethyl-4-methylisoxazol-5-yl)carbamate (184 mg, 0.746 mmol) was added into above solution and followed by addition of DIEA (161 mg, 1.24 mmol). The mixture was stirred at room temperature for over night. The mixture was concentrated to provide crude product, which was purified by flash chromatography (20 to 40% ethyl acetate in heptane) to provide title product as white solid (213 mg, yield 78%). ¹H NMR (400 MHz, DMSO-d₆) δ ppm 1.11-1.21 (m, 4H) 1.57-1.61 (m, 1H) 1.62-1.71 (m, 3H) 1.72-1.81 (m, 4H) 2.30 (dd, J=12.45, 8.05 Hz, 1H) 2.56 (s, 1H) 3.34-3.44 (m, J=13.09, 8.65, 8.65, 4.39 Hz, 2H) 3.50-3.61 (m, 3H) 3.65 (t, J=8.60 Hz, 1H) 4.12 (t, J=7.69 Hz, 1H) 7.31 (dd, J=8.24, 2.38 Hz, 1H) 7.54-7.59 (m, 2H) 9.04 (s, 1H)). m/z (MH⁺) calc 438.1351, found 438.1436.

### Phenyl (3-n-propyl-4-methylisoxazol-5-yl)carbamate

The title compound was prepared from 5-amino-3-n-propyl-4-methylisoxazole (CAS# 909132-91-8) (1.00 g, 7 mmol) using same procedure as described for preparing for phenyl (3-ethyl-4-methylisoxazol-5-yl)carbamate to provide the title compound (0.45 g, 20%). m/z 261.1 (MH⁺).

### Example 69

### 3-(3,4-dichlorophenyl)-N-(3-n-propyl-4-methylisoxazol-5-yl)-1-oxa-8-azaspiro[4.5]decane-8-carboxamide Enantiomer 2

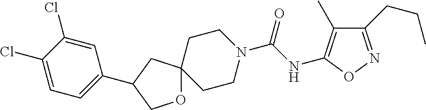

3-(3,4-Dichlorophenyl)-1-oxa-8-azaspiro[4.5]decane hydrochloride Enantiomer 2 (24 mg, 0.075 mmol) was dissolved in acetonitrile (1 mL) in a vial, phenyl (3-n-propyl-4-methylisoxazol-5-yl)carbamate (19.5 mg, 0.075 mmol) was added into above solution and followed by addition of DIEA (29 mg, 0.224 mmol). The mixture was stirred at room temperature overnight. The reaction was concentrated under a stream of nitrogen, dissolved in 1 mL DMSO, and purified by reverse phase HPLC (acetonitrile/water/0.1% formic acid) to give the title compound. ¹H NMR (400 MHz, DMSO-d₆) δ ppm 0.91 (t, J=7.32 Hz, 3H) 1.59 (dt, J=14.83, 7.23 Hz, 4H) 1.67 (d, J=4.03 Hz, 2H) 1.70-1.80 (m, 5H) 2.28 (dd, J=12.45, 7.69 Hz, 1H) 2.46 (m, 2H) 3.38 (dd, J=9.15, 4.03 Hz, 1H) 3.49-3.58 (m, 3H) 3.64 (t, J=8.79 Hz, 1H) 4.11 (t, J=7.50 Hz, 1H) 7.31 (dd, J=8.42, 1.83 Hz, 1H) 7.55 (s, 1H) 7.56-7.63 (m, 1H) 9.09 (s, 1H). m/z (MH⁺) calc 415.1429, found 452.151.

### Phenyl (5-methyl-1,3,4-oxadiazol-2-yl)carbamate

5-Methyl-1,3,4-oxadiazol-2-amine (2.37 g, 23.9 mmol; CAS# 52838-39-8) was added to a 0° C. of phenyl chloroformate in THF (35.5 mL). After 1 h, the reaction was diluted with diethyl ether and filtered to give the title compound as a white solid (6.1 g, quant., ˜90% pure). ¹H NMR (400 MHz, DMSO-d₆) δ ppm 8.41 (br. s., 1H), 7.49 (t, J=8.0 Hz, 2H), 7.29-7.39 (m, 3H), 2.41 (s, 3H). m/z 220 (MH⁺).

### Example 70

### N-(5-methyl-1,3,4-oxadiazol-2-yl)-3-[3-(trifluoromethoxy)phenyl]-1-oxa-8-azaspiro[4.5]decane-8-carboxamide Enantiomer 1

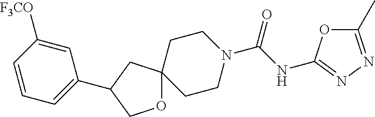

A mixture of 3-[3-(trifluoromethoxy)phenyl]-1-oxa-8-azaspiro[4.5]decane hydrochloride Enantiomer 1 (338 mg, 1.0 mmol) and phenyl (5-methyl-1,3,4-oxadiazol-2-yl)carbamate (438 mg, 2.0 mmol) in acetonitrile (5.6 mL) was treated with DIEA (1.22 mL, 7.00 mmol) and stirred for 2 h at RT. The reaction was concentrated under a stream of nitrogen, and the residue was purified by reverse phase HPLC (acetonitrile/water/0.05% TFA) to give the title compound as a clear gum (88 mg, 20%). ¹H NMR (400 MHz, DMSO-d₆) δ ppm 7.46 (t, J=8.1 Hz, 1H), 7.37 (d, J=8.1 Hz, 1H), 7.31 (br. s., 1H), 7.23 (d, J=8.1 Hz, 1H), 4.15 (t, J=7.3 Hz, 1H), 3.56-3.72 (m, 4H), 3.39-3.47 (m, 2H), 2.38 (s, 3H), 2.32 (dd, J=12.4, 7.3 Hz, 1H), 1.52-1.83 (m, 5H). m/z 427 (MH⁺).

### tert-butyl (3RS)-3-[methylsulfonyl)oxy]-1-oxa-8-azaspiro[4.5]decane-8-carboxylate

A solution of tert-butyl (3RS)-3-hydroxy-1-oxa-8-azaspiro[4.5]decane-8-carboxylate (5.00 g, 19.4 mmol) in dichloromethane (50 mL) at 0° C. was treated with triethylamine (4.06 mL, 29.1 mmol) followed by methanesulfonyl chloride (1.65 mL, 21.4 mmol) dropwise. After 1.5 hr at 0 C, the mixture was washed with satd sodium bicarbonate twice, dried over MgSO4, filtered, and concentrated to give the title compound as an oil (7.54 g, 100%). m/z 236 (MH⁺ minus Boc).

### tert-butyl (3RS)-3-cyano-1-oxa-8-azaspiro[4.5]decane-8-carboxylate

A mixture of tert-butyl (3RS)-3-[(methylsulfonyl)oxy]-1-oxa-8-azaspiro[4.5]decane-8-carboxylate (6.71 g, 20.0 mmol), sodium cyanide (1.15 g, 30.8 mmol), and sodium iodide (300 mg, 0.10 mmol) in DMF (50 mL) was heated with stirring at 90° C. for 6 days. The reaction was treated with EA and sat. sodium bicarbonate and extracted with EA (3×100 mL). The organic was dried over magnesium sulfate, filtered and evaporated to give ˜10 g of an oil. The oil was diluted with water, and extracted with EA (200 mL). The organic was washed with 0.1N HCl (4×100 mL), dried over magnesium sulfate, filtered and evaporated to give about 5.5 g of a dark oil. The oil was purified by flash chromatography with 2-25% EA/Heptane to give the title compound as an oil (1.3 g, 25%). m/z 167 (MH⁺ minus Boc) ¹H NMR (400 MHz, CDCl₃) δ ppm 1.45 (s, 9H), 1.51-1.70 (m, 3H), 1.74-1.84 (m, 1H), 2.01-2.18 (m, 2H), 3.10-3.31 (m, 3H), 3.69 (br. s., 2H), 3.98-4.05 (m, 1H), 4.07-4.16 (m, 1H).

### tert-butyl (3RS)-3-[amino(hydroxyimino)methyl]-1-oxa-8-azaspiro[4.5]decane-8-carboxylate

A solution of tert-butyl (3RS)-3-cyano-1-oxa-8-azaspiro[4.5]decane-8-carboxylate (1.32 g, 4.96 mmol) in EtOH (10 mL) was treated with hydroxylamine hydrochloride (1.03 g, 14.9 mmol) and triethylamine (2.21 mL, 15.9 mmol). The reaction was heated with stirring at 55° C. overnight. To the reaction was added triethylamine (1.1 mL, 7.89 mmol) and hydroxylamine hydrochloride (500 mg, 7.19 mmol). The reaction was heated with stirring at 55° C. overnight. To the reaction was added triethylamine (1.1 mL, 7.89 mmol) and hydroxylamine hydrochloride (500 mg, 7.19 mmol). The reaction was heated with stirring at 55° C. over a third night. The reaction was diluted with ethyl acetate and washed with water. The organic was dried over magnesium sulfate, filtered and evaporated to give the title compound as a foam (1.48 g, 100%). m/z 244 (MH⁺ minus tBu). ¹H NMR (400 MHz, DMSO-d₆) δ ppm 1.38 (s, 9H), 1.40-1.56 (m, 4H), 1.77-1.86 (m, 1H), 1.95 (dd, J=12.46, 8.36 Hz, 1H), 2.85-2.96 (m, 1H), 3.22 (br. s., 2H), 3.37-3.49 (m, 2H), 3.71 (t, J=8.70 Hz, 1H), 3.90 (t, J=8.02 Hz, 1H), 5.39 (s, 2H), 8.92 (s, 1H).

### Example 71

### (3RS)—N-(3,4-dimethylisoxazol-5-yl)-3-{5-[4-(trifluoromethoxy)phenyl]-1,2,4-oxadiazol-3-yl}-1-oxa-8-azaspiro[4.5]decane-8-carboxamide

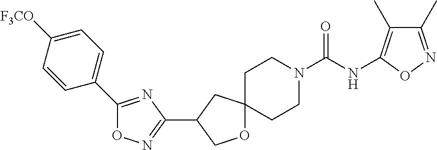

A solution of tert-butyl (3RS)-3-[amino(hydroxyimino)methyl]-1-oxa-8-azaspiro[4.5]decane-8-carboxylate (22 mg, 0.73 mmol) in THF (0.5 mL) was added DIEA (0.050 mL, 0.29 mmol) and the 4-(trifluoromethoxy)benzoyl chloride (0.012 mL, 0.073 mmol) at RT. The reaction vessel was capped and heated to 150° C. in a CEM discover microwave for 30 min. The solution was evaporate to give the crude oil. The oil was dissolved in DCM (1 mL) and treated with 4N HCl/dioxane (0.5 mL). The reaction mixture was stirred for 1 hr at RT and then evaporated. To the residue and phenyl (3,4-dimethylisoxazol-5-yl)carbamate (17 mg, 0.073 mmol) was added acetonitrile (4 mL), DIEA (0.40 mL, 2.0 mmol) and the reaction was stirred for 4 hrs. The reaction was concentrated under a stream of nitrogen, dissolved in 1 mL DMSO, and purified by reverse phase HPLC (acetonitrile/water/0.1°)/0 formic acid) to give the title compound (12.1 mg, 36%). m/z 508.181 (MH⁺).

### tert-butyl 3-{5-[4-(trifluoromethoxy)phenyl]-1,2,4-oxadiazol-3-yl}-1-oxa-8-azaspiro[4.5]decane-8-carboxylate Enantiomers 1 and 2

A solution of tert-butyl (3RS)-3-[amino(hydroxyimino)methyl]-1-oxa-8-azaspiro[4.5]decane-8-carboxylate (650 mg, 2.17 mmol) in THF (10 mL) was added DIEA (1.51 mL, 8.68 mmol) and the 4-(trifluoromethoxy)benzoyl chloride (488 mg, 2.17 mmol) at room temperature. The reaction vessel was capped and heated to 150° C. in a CEM discover microwave for 30 min. The solution was diluted with ethyl acetate (100 mL) and washed with water (50 mL), sat. sodium bicarbonate (50 mL), and brine (50 mL). The layers were separated and the organic was dried over magnesium sulfate, filtered and rotovapped to give the racemic title compound as a solid (990 mg, 97%), which was separated by chiral SFC on a 30×250 mm ChiralPak AD-H column (50% MeOH/CO₂ @ 70 mL/min; 2 mL injections of a 15 mg/mL MeOH/DCM solution). The first eluting peak gave Enantiomer 1 of the title compound (324 mg; t_{R}=0.81 min (4.6×100 mm Chiralpak AD-H, 50% MeOH/CO₂ at 3 mL/min)) and the second eluting peak gave Enantiomer 2 of the title compound (375 mg; t_{R}=2.61 min (4.6×100 mm Chiralpak AD-H, 50% MeOH/CO₂ at 3 ml/min)). m/z 370 (MH⁺ minus Boc).

### 3-{5-[4-(trifluoromethoxy)phenyl]-1,2,4-oxadiazol-3-yl}-1-oxa-8-azaspiro[4.5]decane hydrochloride Enantiomer 1

To tert-butyl 3-{5-[4-(trifluoromethyl)phenyl]-1,2,4-oxadiazol-3-yl}-1-oxa-8-azaspiro[4.5]decane-8-carboxylate Enantiomer 1 (324 mg, 0.690 mmol) was added DCM (2 mL) followed by 4N HCl/dioxane (2 mL). The solution was stirred for 4 hr at RT and then evaporated to give the title compound as a solid (314 mg, quant.). m/z 370 (MH⁺).

### Example 72

### N-(3,4-dimethylisoxazol-5-yl)-3-{5-[4-(trifluoromethoxy)phenyl]-1,2,4-oxadiazol-3-yl}-1-oxa-8-azaspiro[4.5]decane-8-carboxamide Enantiomer 1

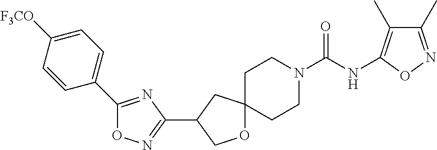

A mixture of 3-{5-[4-(trifluoromethoxy)phenyl]-1,2,4-oxadiazol-3-yl}-1-oxa-8-azaspiro[4.5]decane hydrochloride Enantiomer 1 (200 mg, 0.493 mmol) and phenyl (3,4-dimethylisoxazol-5-yl)carbamate (114 mg, 0.493 mmol) in acetonitrile (4 mL) was treated with DIEA (0.40 mL, 2.0 mmol) and stirred overnight. The mixture was evaporated and the crude residue was purified by flash chromatography (50-100% EA/Heptane) to give the title compound as a solid (139 mg, 56%). m/z 508 (MH⁺). ¹H NMR (400 MHz, DMSO-d₆) δ ppm 1.56-1.71 (m, 4H), 1.74 (s, 3H), 2.04-2.14 (m, 4H), 2.33 (dd, J=12.63, 8.88 Hz, 1H), 3.30-3.40 (m, 2H), 3.55-3.67 (m, 2H), 3.79-3.90 (m, 1H), 3.96 (t, J=8.02 Hz, 1H), 4.22 (t, J=8.19 Hz, 1H), 7.63 (d, J=8.88 Hz, 2H), 8.24 (d, J=8.53 Hz, 2H), 9.12 (s, 1H).

### 3-{5-[4-(trifluoromethoxy)phenyl]-1,2,4-oxadiazol-3-yl}-1-oxa-8-azaspiro[4.5]decane hydrochloride Enantiomer 2

To tert-butyl 3-{5-[4-(trifluoromethyl)phenyl]-1,2,4-oxadiazol-3-yl}-1-oxa-8-azaspiro[4.5]decane-8-carboxylate Enantiomer 2 (375 mg, 0.799 mmol) was added DCM (2 mL) followed by 4N HCl/dioxane (2 mL). The solution was stirred for 4 hr at RT and then evaporated to give the title compound as a solid (290 mg, 90%). m/z 370 (MH⁺).

### Example 73

### N-(3,4-dimethylisoxazol-5-yl)-3-{5-[4-(trifluoromethoxy)phenyl]-1,2,4-oxadiazol-3-yl}-1-oxa-8-azaspiro[4.5]decane-8-carboxamide Enantiomer 2

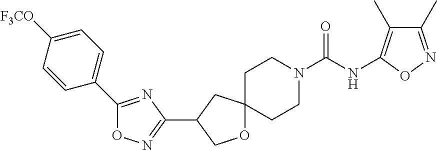

The title compound was prepared from 3-{5-[4-(trifluoromethoxy)phenyl]-1,2,4-oxadiazol-3-yl}-1-oxa-8-azaspiro[4.5]decane hydrochloride Enantiomer 2 (200 mg, 0.493 mmol) as described for Example 72 to give the title compound as a solid (183 mg, 73%). m/z 508 (MH⁺). ¹H NMR (400 MHz, DMSO-d₆) δ ppm 1.55-1.71 (m, 4H), 1.73 (s, 3H), 2.02-2.14 (m, 4H), 2.32 (dd, J=12.46, 8.70 Hz, 1H), 3.29-3.39 (m, 2H), 3.55-3.69 (m, 2H), 3.79-3.90 (m, 1H), 3.91-4.00 (m, 1H), 4.21 (t, J=8.19 Hz, 1H), 7.62 (d, J=8.19 Hz, 2H), 8.23 (d, J=8.88 Hz, 2H), 9.12 (s, 1H).

### tert-butyl (3RS)-3-{5-[4-(trifluoromethyl)phenyl]-1,2,4-oxadiazol-3-yl}-1-oxa-8-azaspiro[4.5]decane-8-carboxylate

The title compound was prepared from tert-butyl (3RS)-3-[amino(hydroxyimino)methyl]-1-oxa-8-azaspiro[4.5]decane-8-carboxylate (650 mg, 2.17 mmol) and 4-(trifluoromethyl)benzoyl chloride (0.356 mL, 2.39 mmol) as described for tert-butyl (3RS)-3-{5-[4-(trifluoromethoxy)phenyl]-1,2,4-oxadiazol-3-yl}-1-oxa-8-azaspiro[4.5]decane-8-carboxylate to give the title compound as a solid (933 mg, 95%). m/z 354 (MH⁺ minus Boc).

### Example 74

### (3RS)—N-(3,4-dimethylisoxazol-5-yl)-3-{5-[4-(trifluoromethyl)phenyl]-1,2,4-oxadiazol-3-yl}-1-oxa-8-azaspiro[4.5]decane-8-carboxamide

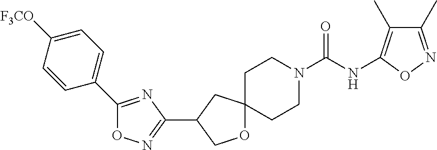

To tert-butyl (3RS)-3-{5-[4-(trifluoromethyl)phenyl]-1,2,4-oxadiazol-3-yl}-1-oxa-8-azaspiro[4.5]decane-8-carboxylate (21 mg, 0.046 mmol) dissolved in DCM (0.5 mL) was added 4N HCl/dioxane (0.5 mL). The reaction mixture was stirred overnight and then evaporated. To the residue and phenyl (3,4-dimethylisoxazol-5-yl)carbamate (11 mg, 0.046 mmol) was added acetonitrile (1 mL), DIEA (0.032 mL, 0.18 mmol) and the reaction was stirred overnight. The reaction was concentrated under a stream of nitrogen, dissolved in 1 mL DMSO, and purified by reverse phase HPLC (acetonitrile/water/0.1°)/0 formic acid) to give the title compound (12 mg, 52%). m/z 492.186 (MH⁺).

### tert-butyl 3-{5-[4-(trifluoromethyl)phenyl]-1,2,4-oxadiazol-3-yl}-1-oxa-8-azaspiro[4.5]decane-8-carboxylate Enantiomers 1 and 2

The racemic tert-butyl (3RS)-3-{5-[4-(trifluoromethyl)phenyl]-1,2,4-oxadiazol-3-yl}-1-oxa-8-azaspiro[4.5]decane-8-carboxylate (913 mg, 2.01 mmol) was separated by chiral SFC on a 30×250 mm ChiralPak AD-H column (50% MeOH/CO₂ @ 70 mL/min; 5 mL injections of a 15 mg/mL MeOH/DCM solution). The first eluting peak gave Enantiomer 1 of the title compound (345 mg; t_{R}=0.90 min (4.6×100 mm Chiralpak AD-H, 50% MeOH/CO₂ at 3 mL/min)) and the second eluting peak gave Enantiomer 2 of the title compound (294 mg; t_{R}=2.38 min (4.6×100 mm Chiralpak AD-H, 50% MeOH/CO₂ at 3 ml/min)). m/z 354 (MH⁺ minus Boc).

### 3-{5-[4-(trifluoromethyl)phenyl]-1,2,4-oxadiazol-3-yl}-1-oxa-8-azaspiro[4.5]decane hydrochloride Enantiomer 1

The title compound was prepared from tert-butyl 3-{5-[4-(trifluoromethyl)phenyl]-1,2,4-oxadiazol-3-yl}-1-oxa-8-azaspiro[4.5]decane-8-carboxylate Enantiomer 1 (345 mg, 0.761 mmol) as described for 3-{5-[4-(trifluoromethoxy)phenyl]-1,2,4-oxadiazol-3-yl}-1-oxa-8-azaspiro[4.5]decane Enantiomer 1 to give the title compound as a solid (317 mg, quant.). m/z 354 (MH⁺).

### Example 75

### N-(3,4-dimethylisoxazol-5-yl)-3-{5-[4-(trifluoromethyl)phenyl]-1,2,4-oxadiazol-3-yl}-1-oxa-8-azaspiro[4.5]decane-8-carboxamide Enantiomer 1

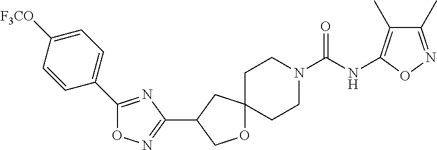

The title compound was prepared from 3-{5-[4-(trifluoromethyl)phenyl]-1,2,4-oxadiazol-3-yl}-1-oxa-8-azaspiro[4.5]decane hydrochloride Enantiomer 1 (200 mg, 0.513 mmol) as described for Example 72 except that the chromatography fractions were concentrated to a small volume (˜10 mL) to give crystals upon standing. The crystals were collected by vacuum filtration and dried on the high vac to give the title compound (134 mg, 53%). The filtrate was allowed to slowly evaporate to a small volume to give a second crop of the title compound as a solid (60 mg, 24%). m/z 492 (MH⁺). ¹H NMR (400 MHz, DMSO-d₆) δ ppm 1.57-1.72 (m, 4H), 1.74 (s, 3H), 2.05-2.11 (m, 1H), 2.12 (s, 3H), 2.34 (dd, J=12.80, 8.70 Hz, 1H), 3.31-3.40 (m, 2H), 3.55-3.67 (m, 2H), 3.81-3.92 (m, 1H), 3.94-4.01 (m, 1H), 4.23 (t, J=8.19 Hz, 1H), 8.01 (d, J=8.19 Hz, 2H), 8.32 (d, J=8.19 Hz, 2H), 9.13 (s, 1H).

### 3-{5-[4-(trifluoromethyl)phenyl]-1,2,4-oxadiazol-3-yl}-1-oxa-8-azaspiro[4.5]decane hydrochloride Enantiomer 2

The title compound was prepared from tert-butyl 3-{5-[4-(trifluoromethyl)phenyl]-1,2,4-oxadiazol-3-yl}-1-oxa-8-azaspiro[4.5]decane-8-carboxylate Enantiomer 2 (294 mg, 0.648 mmol) as described for 3-{5-[4-(trifluoromethoxy)phenyl]-1,2,4-oxadiazol-3-yl}-1-oxa-8-azaspiro[4.5]decane Enantiomer 1 to give the title compounds as a solid (270 mg, quant.). m/z 354 (MH⁺)

### Example 76

### N-(3,4-dimethylisoxazol-5-yl)-3-{5-[4-(trifluoromethyl)phenyl]-1,2,4-oxadiazol-3-yl}-1-oxa-8-azaspiro[4.5]decane-8-carboxamide Enantiomer 2

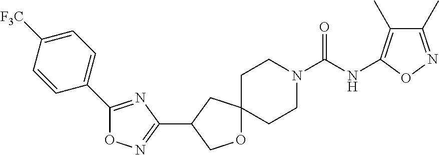

The title compound was prepared from 3-{5-[4-(trifluoromethyl)phenyl]-1,2,4-oxadiazol-3-yl}-1-oxa-8-azaspiro[4.5]decane hydrochloride Enantiomer 2 (200 mg, 0.513 mmol) as described for Example 75 give the title compound (73 mg, 29%) and a second crop of the title compound as a solid (139 mg, 55%). m/z 492 (MH⁺). ¹H NMR (400 MHz, DMSO-d₆) δ ppm 1.58-1.73 (m, 4H), 1.74 (s, 3H), 2.05-2.11 (m, 1H), 2.12 (s, 3H), 2.34 (dd, J=12.63, 8.88 Hz, 1H), 3.31-3.40 (m, 2H), 3.56-3.67 (m, 2H), 3.82-3.93 (m, 1H), 3.98 (t, J=8.02 Hz, 1H), 4.23 (t, J=8.19 Hz, 1H), 8.01 (d, J=8.53 Hz, 2H), 8.32 (d, J=8.53 Hz, 2H), 9.13 (s, 1H).

### 4-nitrophenyl 3-[3-(trifluoromethoxy)phenyl]-1-oxa-8-azaspiro[4.5]decane-8-carboxylate Enantiomer 1

The title compound was prepared from 3-[3-(trifluoromethoxy)phenyl]-1-oxa-8-azaspiro[4.5]decane hydrochloride Enantiomer 1 (500 mg, 1.48 mmol) as described for 4-nitrophenyl 3-[3-(trifluoromethyl)phenyl]-1-oxa-8-azaspiro[4.5]decane-8-carboxylate Enantiomer 1 (see Example 92) to provide title compound (690 mg, quant.). m/z 467.2 (MH⁺).

### Example 77

### N-(1-methyl-1H-tetrazol-5-yl)-3-[3-(trifluoromethoxy)phenyl]-1-oxa-8-azaspiro[4.5]decane-8-carboxamide Enantiomer 1

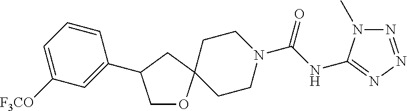

The title compound was prepared from 4-nitrophenyl 3-[3-(trifluoromethoxy)phenyl]-1-oxa-8-azaspiro[4.5]decane-8-carboxylate Enantiomer 1 (450 mg, 0.965 mmol) as described for Example 93 to provide the title compound (125 mg, 30.4% yield). ¹H NMR (400 MHz, methanol-d₄) δ ppm 1.60-1.63 (m, 1H) 1.67-1.76 (m, 3H) 1.76-1.78 (m, 1H) 2.29-2.34 (m, 1H) 3.56-3.62 (m, 3H) 3.65 (br. s., 3H) 3.75 (t, J=8 Hz, 1H) 3.82-3.90 (m, 2H) 4.18 (t, J=8 Hz, 1H) 7.07-7.09 (d, J=8 Hz, 1H) 7.16 (s, 1H) 7.26-7.28 (d, J=8 Hz, 1 H) 7.36 (t, J=8 Hz, 1H). m/z 427.2 (MH⁺).

### 4-nitrophenyl 3-[3-(trifluoromethoxy)phenyl]-1-oxa-8-azaspiro[4.5]decane-8-carboxylate Enantiomer 2

The title compound was prepared from 3-[3-(trifluoromethoxy)phenyl]-1-oxa-8-azaspiro[4.5]decane hydrochloride Enantiomer 2 (352 mg, 1.04 mmol) as described for 4-nitrophenyl 3-[3-(trifluoromethyl)phenyl]-1-oxa-8-azaspiro[4.5]decane-8-carboxylate Enantiomer 1 to provide title compound (486 mg, quant.). m/z 467.2 (MH⁺).

### Example 78

### N-(1-methyl-1H-tetrazol-5-yl)-3-[3-(trifluoromethoxy)phenyl]-1-oxa-8-azaspiro[4.5]decane-8-carboxamide Enantiomer 2

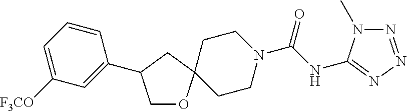

The title compound was prepared from 4-nitrophenyl 3-[3-(trifluoromethoxy)phenyl]-1-oxa-8-azaspiro[4.5]decane-8-carboxylate Enantiomer 2 (480 mg, 1.03 mmol) as described for Example 93 to provide the title compound (125 mg, 34% yield). ¹H NMR (400 MHz, DMSO-d₆) δ ppm 1.57-1.63 (m, 2H) 1.76-1.82 (m, 3H) 2.21-2.33 (m, 1H) 3.38-3.47 (m, 2H) 3.53-3.66 (m, 3H) 3.67 (s, 1H) 3.72-3.83 (m, 3 H) 4.06-4.16 (m, 1H) 7.25-7.30 (m, 2H) 7.31-7.38 (m, 2H) 9.92 (d, J=7.69 Hz, 1H). m/z (MH⁺) calc 427.1705, found 427.1988.

### 4-nitrophenyl 3-(3-chlorophenol)-1-oxa-8-azaspiro[4.5]decane-8-carboxylate Enantiomer 1

The title compound was prepared from 3-(3-chlorophenyl)-1-oxa-8-azaspiro[4.5]decane hydrochloride Enantiomer 1 (427 mg, 1.48 mmol) as described for 4-nitrophenyl 3-[3-(trifluoromethyl)phenyl]-1-oxa-8-azaspiro[4.5]decane-8-carboxylate Enantiomer 1 to provide title compound (617 mg, quant.). m/z 417.2 (MH⁺).

### Example 79

### 3-(3-chlorophenyl)-N-(1-methyl-1H-tetrazol-5-yl)-1-oxa-8-azaspiro[4.5]decane-8-carboxamide Enantiomer 1

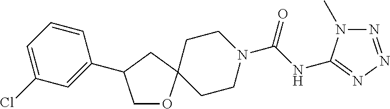

The title compound was prepared from 4-nitrophenyl 3-(3-chlorophenyl)-1-oxa-8-azaspiro[4.5]decane-8-carboxylate Enantiomer 1 (400 mg, 0.96 mmol) as described for Example 93 to provide the title compound (175 mg, 48.4%). ¹H NMR (400 MHz, methanol-d₄) δ ppm 1.23 (t, J=7.13 Hz, 1H) 1.60-1.92 (m, 5H) 2.01 (s, 2H) 2.34 (dd, J=12.60, 8.11 Hz, 1H) 3.57 (dt, J=17.09, 8.45 Hz, 3H) 3.68-3.95 (m, 6H) 4.09 (q, J=7.03 Hz, 1H) 4.21 (t, J=7.91 Hz, 1H) 7.15-7.38 (m, 4H). m/z 377.0 (MH⁺).

### 4-nitrophenyl 3-(3-chlorophenyl)-1-oxa-8-azaspiro[4.5]decane-8-carboxylate Enantiomer 2

The title compound was prepared from 3-(3-chlorophenyl)-1-oxa-8-azaspiro[4.5]decane hydrochloride Enantiomer 2 (300 mg, 1.04 mmol) as described for 4-nitrophenyl 3-[3-(trifluoromethyl)phenyl]-1-oxa-8-azaspiro[4.5]decane-8-carboxylate Enantiomer 1 to provide title compound (434 mg, quant.). m/z 417.2 (MH⁺).

### Example 80

### 3-(3-chlorophenyl)-N-(1-methyl-1H-tetrazol-5-yl)-1-oxa-8-azaspiro[4.5]decane-8-carboxamide Enantiomer 2

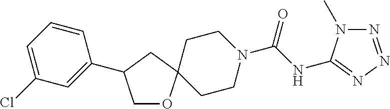

The title compound was prepared from 4-nitrophenyl 3-(3-chlorophenyl)-1-oxa-8-azaspiro[4.5]decane-8-carboxylate Enantiomer 2 (450 mg, 1.08 mmol) as described for Example 93 to provide the title compound (95 mg, 23% yield). ¹H NMR (400 MHz, DMSO-d₆) δ ppm 1.58-1.68 (m, 2H) 1.68-1.76 (m, 2H) 2.21-2.33 (m, 1H) 3.38-3.47 (m, 2H) 3.53-3.66 (m, 4H) 3.67 (m, 1H) 3.78 (s, 3H) 4.09-4.14 (m, 1H) 7.25-7.30 (m, 2H) 7.31-7.38 (m, 2H) 9.92 (d, J=7.69 Hz, 1H). m/z (MH⁺) calc 377.1493, found 377.1592.

### 4-nitrophenyl (3R)-3-{3-[(5-chloropyridin-2-yl)oxy]phenyl}-1-oxa-8-azaspiro[4.5]decane-8-carboxylate

The title compound was prepared from (3R)-3-{3-[(5-chloropyridin-2-yl)oxy]phenyl}-1-oxa-8-azaspiro[4.5]decane hydrochloride (1200 mg, 2.87 mmol) as described for 4-nitrophenyl 3-[3-(trifluoromethyl)phenyl]-1-oxa-8-azaspiro[4.5]decane-8-carboxylate Enantiomer 1 to provide title compound (1300 mg, 89% yield). m/z 510.2 (MH⁺).

### Example 81

### 3-{3-[(5-chloropyridin-2-yl)oxy]phenyl}-N-(1-methyl-1H-tetrazol-5-yl)-1-oxa-8-azaspiro[4.5]decane-8-carboxamide

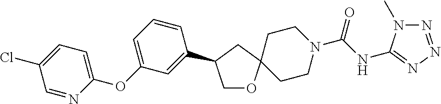

The title compound was prepared from 4-nitrophenyl (3R)-3-{3-[(5-chloropyridin-2-yl)oxy]phenyl}-1-oxa-8-azaspiro[4.5]decane-8-carboxylate (500 mg, 0.98 mmol) as described for Example 93 to provide the title compound (275 mg, 59.7% yield). ¹H NMR (400 MHz, DMSO-d₆) δ ppm 1.62 (m, 2H) 1.85 (m, 2H) 2.20-2.34 (m, 2H) 3.56 (m, 3H) 3.82 (m, 1H) 3.97 (s, 3H) 3.97-4.10 (m, 2H) 4.25 (t, J=7.62 Hz, 1H) 6.89 (d, J=8.40 Hz, 1H) 7.00 (br. s., 2H) 7.10 (d, J=6.64 Hz, 1H) 7.36 (t, J=7.33 Hz, 1H) 7.60-7.71 (m, 1H) 8.13 (br. s., 1H) 10.51 (br. s., 1H). m/z (MH⁺) calc 470.1707 fnd. 470.1760.

### Example 82

### N-(1-methyl-1H-tetrazol-5-yl)-3-{5-[4-(trifluoromethyl)phenyl]-1,2,4-oxadiazol-3-yl}-1-oxa-8-azaspiro[4.5]decane-8-carboxamide Enantiomer 1

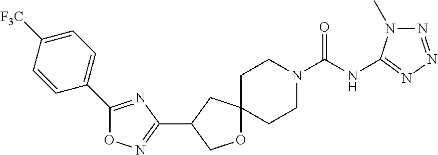

The title compound was prepared from 3-{5-[4-(trifluoromethyl)phenyl]-1,2,4-oxadiazol-3-yl}-1-oxa-8-azaspiro[4.5]decane hydrochloride Enantiomer 1 (101 mgs, 0.286 mmol) as described for Example 84. The crude product was purified by flash chromatography (ethyl acetate(5% ethanol)/heptane) to produce the title compound as a light yellow solid. (81 mg, 0.169 mmol, 60%). ¹H NMR (600 MHz, DMSO-d₆) δ ppm 9.92 (1H, br. s.), 8.33 (2H, d, J=8.0 Hz), 8.03 (2H, d, J=8.2 Hz), 4.26 (1H, t, J=8.1 Hz), 4.02 (1H, t, J=7.8 Hz), 3.90 (1 H, s), 3.70 (3H, t, J=14.8 Hz), 3.42 (3H, t, J=9.8 Hz), 2.52 (3H, br. s.), 2.34-2.40 (1H, m), 2.10-2.17 (1H, m), 1.70 (1H, br. s.). m/z 478.2 (MH⁺).

### Example 83

### N-(1-methyl-1H-tetrazol-5-yl)-3-{5-[4-(trifluoromethyl)phenyl]-1,2,4-oxadiazol-3-yl}-1-oxa-8-azaspiro[4.5]decane-8-carboxamide Enantiomer 2

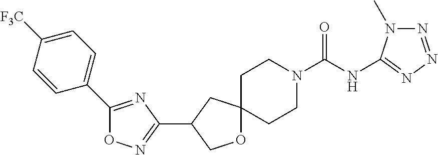

The title compound was prepared from 3-{5-[4-(trifluoromethyl)phenyl]-1,2,4-oxadiazol-3-yl}-1-oxa-8-azaspiro[4.5]decane hydrochloride Enantiomer 2 (55 mg, 0.160 mmol) as described for Example 84. The crude product was purified by flash chromatography (ethyl acetate(5% ethanol)/heptane) to produce the title compound as a light yellow solid. (54 mg, 0.112 mmol, 72%) ¹H NMR (600 MHz, DMSO-d₆) δ ppm 9.92 (1H, br. s.), 8.33 (2H, d, J=8.0 Hz), 8.03 (2H, d, J=8.2 Hz), 4.26 (1H, t, J=8.1 Hz), 4.02 (1H, t, J=7.8 Hz), 3.90 (1 H, s), 3.70 (3H, t, J=14.8 Hz), 3.42 (3H, t, J=9.8 Hz), 2.52 (3H, br. s.), 2.34-2.40 (1H, m), 2.10-2.17 (1H, m), 1.70 (1H, br. s.). m/z 478.2 (MH⁺).

### Example 84

### N-(1-methyl-1H-tetrazol-5-yl)-3-{5-[4-(trifluoromethoxy)phenyl]-1,2,4-oxadiazol-3-yl}-1-oxa-8-azaspiro[4.5]decane-8-carboxamide Enantiomer 1

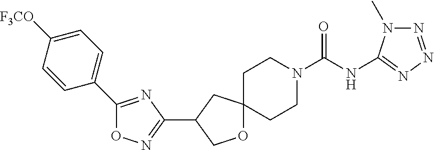

3-{5-[4-(trifluoromethoxy)phenyl]-1,2,4-oxadiazol-3-yl}-1-oxa-8-azaspiro[4.5]decane hydrochloride Enantiomer 1 (126 mg, 0.358 mmol) was dissolved in dioxane (1 mL), and satd sodium bicarbonate (1 mL) and stirred for 10 minutes at r.t. A solution of the nitrophenyl chloroformate (72.2 mg, 0.358 mmol) in dioxane (1 mL) was added dropwise slowly and the reaction was stirred at room temp for 2 h. The reaction was then diluted with water (2 mL) and the aqueous phase extracted with ethyl acetate (2×5 mL), the organics dried with magnesium sulfate and concentrated to provide the crude nitrophenyl carbamate. 1-methyl-1H-tetrazol-5-amine (46.8 mg, 0.472 mmol) was dissolved in DMA (0.5 mL) at r.t. and sodium hydride (19.8 mg, 0.496 mmol) added. The reaction was stirred for 15 minutes at which time a solution of the crude nitrophenyl carbamate in DMA (1 mL) was added dropwise. The reaction was stirred for 1 hour and then quenched with sat. sodium bicarbonate. The reaction was extracted with ethyl acetate (3×10 mL), dried with magnesium sulfate and concentrated. The crude product was purified by flash chromatography (ethyl acetate(5% ethanol)/heptane) to produce the title compound as a light yellow solid. (98 mg, 0.198 mmol, 84%) ¹H NMR (600 MHz, DMSO-d₆) δ ppm 9.92 (1H, br. s.), 8.26 (2H, d, J=8.8 Hz), 7.64 (2H, d, J=8.2 Hz), 4.25 (1H, t, J=8.1 Hz), 4.00 (1H, t, J=8.0 Hz), 3.84-3.91 (1H, m), 3.65-3.75 (3H, m), 3.42 (3H, t, J=9.8 Hz), 2.52 (3H, br. s.), 2.36 (1H, dd, J=12.6, 8.8 Hz), 2.12 (1H, dd, J=12.6, 8.0 Hz), 1.65-1.73 (2H, m). m/z 494.9 (MH⁺).

### Example 85

### N-(1-methyl-1H-tetrazol-5-yl)-3-{5-[4-(trifluoromethoxy)phenyl]-1,2,4-oxadiazol-3-yl}-1-oxa-8-azaspiro[4.5]decane-8-carboxamide Enantiomer 2

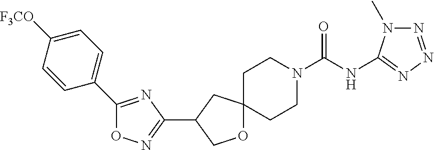

The title compound was prepared from 3-{5-[4-(trifluoromethoxy)phenyl]-1,2,4-oxadiazol-3-yl}-1-oxa-8-azaspiro[4.5]decane hydrochloride Enantiomer 2 (77.0 mg, 0.210 mmol) as described for Example 84. The crude product was purified by flash chromatography (ethyl acetate(5% ethanol)/heptane) to produce the title compound as a light yellow solid. (61 mg, 0.123 mmol, 86%). ¹H NMR (600 MHz, DMSO-d₆) δ ppm 9.92 (1H, br. s.), 8.26 (2H, d, J=8.8 Hz), 7.64 (2H, d, J=8.2 Hz), 4.25 (1H, t, J=8.1 Hz), 4.00 (1H, t, J=8.0 Hz), 3.84-3.91 (1H, m), 3.65-3.75 (3H, m), 3.42 (3H, t, J=9.8 Hz), 2.52 (3H, br. s.), 2.36 (1H, dd, J=12.6, 8.8 Hz), 2.12 (1H, dd, J=12.6, 8.0 Hz), 1.65-1.73 (2H, m). m/z 494.2 (MH⁺).

### Example 86

### (3R)-3-{4-[(3-fluorobenzyl)oxy]phenyl}-N-(1-methyl-1H-tetrazol-5-yl)-1-oxa-8-azaspiro[4.5]decane-8-carboxamide

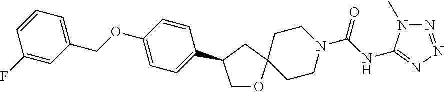

The title compound was prepared from (3R)-3-{4-[(3-fluorobenzyl)oxy]phenyl}-1-oxa-8-azaspiro[4.5]decane hydrochloride (115 mg, 0.337 mmol) as described for Example 84. The crude product was purified by flash chromatography (ethyl acetate(5% ethanol)/heptane) to produce the title compound as a light yellow solid. (60 mg, 0.128 mmol, 38%). ¹H NMR (600 MHz, DMSO-d₆) δ ppm 7.42-7.48 (1H, m), 7.26-7.31 (2H, m), 7.24 (2H, d, J=8.5 Hz), 7.16 (1H, t, J=8.7 Hz), 6.98 (2H, d, J=8.5 Hz), 5.13 (2H, s), 4.12 (1H, t, J=7.8 Hz), 3.82 (2H, s), 3.59-3.70 (3H, m), 3.40-3.54 (3H, m), 2.28 (1H, dd, J=12.1, 8.0 Hz), 1.68-1.82 (6H, m), 1.64 (1H, none). m/z 467.3 (MH⁺).

### Example 87

### (3S)-3-{4-[(3-fluorobenzyl)oxy]phenyl}-N-(1-methyl-1H-tetrazol-5-yl)-1-oxa-8-azaspiro[4.5]decane-8-carboxamide

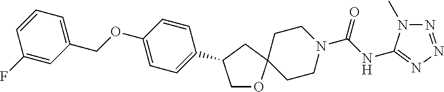

The title compound was prepared from (3S)-3-{4-[(3-fluorobenzyl)oxy]phenyl}-1-oxa-8-azaspiro[4.5]decane hydrochloride (79.0 mg, 0.230 mmol) as described for Example 84. The crude product was purified by flash chromatography (ethyl acetate(5% ethanol)/heptane) to produce the title compound as a light yellow solid. (30 mg, 0.064 mmol, 29%). ¹H NMR (600 MHz, DMSO-d₆) δ ppm 7.42-7.48 (1H, m), 7.26-7.31 (2H, m), 7.24 (2H, d, J=8.5 Hz), 7.16 (1H, t, J=8.7 Hz), 6.98 (2H, d, J=8.5 Hz), 5.13 (2H, s), 4.12 (1H, t, J=7.8 Hz), 3.82 (2H, s), 3.59-3.70 (3H, m), 3.40-3.54 (3H, m), 2.28 (1H, dd, J=12.1, 8.0 Hz), 1.68-1.82 (6H, m), 1.64 (1 H, none). m/z 467.3 (MH⁺).

### Example 88

### (3R)-3-{4-[(3,4-difluorobenzyl)oxy]phenyl}-N-(1-methyl-1H-tetrazol-5-yl)-1-oxa-8-azaspiro[4.5]decane-8-carboxamide

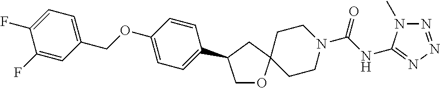

The title compound was prepared from (3R)-3-{4-[(3,4-difluorobenzyl)oxy]phenyl}-1-oxa-8-azaspiro[4.5]decane hydrochloride as described for Example 84. The crude product was purified by reverse phase HPLC (10 to 95% acetonitrile/water/0.05% TFA). The pure fractions were concentrated, dissolved in ethyl acetate, and washed with 0.1 N NaOH and brine, dried over sodium sulfate, filtered, and concentrated to give an oil which was triturated with ethyl acetate to give the title compound as a white solid (329 mg). ¹H NMR (600 MHz, DMSO-d₆) δ ppm 9.91 (br. s., 1H), 7.41-7.55 (m, 2H), 7.27-7.32 (m, 1H), 7.22 (d, J=8.6 Hz, 2H), 6.95 (d, J=8.8 Hz, 2H), 5.07 (s, 2H), 4.10 (t, J=7.7 Hz, 1H), 3.79 (s, 3H), 3.58-3.69 (m, 3H), 3.37-3.52 (m, 3H), 2.26 (dd, J=12.5, 7.8 Hz, 1H), 1.57-1.80 (m, 5H). m/z 485 (MH⁺).

### Example 89

### (3S)-3-{4-[(3,4-difluorobenzyl)oxy]phenyl}-N-(1-methyl-1H-tetrazol-5-yl)-1-oxa-8-azaspiro[4.5]decane-8-carboxamide

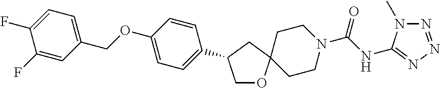

The title compound was prepared from (3S)-3-{4-[(3,4-difluorobenzyl)oxy]phenyl}-1-oxa-8-azaspiro[4.5]decane (100.0 mg, 0.278 mmol) as described for Example 84. The crude product was purified by flash chromatography (ethyl acetate(5% ethanol)/heptane) to produce the title compound as a light yellow solid. (80 mg, 0.165 mmol, 59%). ¹H NMR (600 MHz, DMSO-d₆) δ ppm 7.49-7.56 (1H, m), 7.42-7.49 (1H, m), 7.31 (1H, br. s.), 7.24 (2H, d, J=8.5 Hz), 6.97 (2H, d), 5.09 (2H, s), 4.12 (1H, t, J=7.8 Hz), 3.82 (2H, s), 3.60-3.70 (3H, m), 3.40-3.55 (3H, m), 2.28 (1H, dd, J=12.1, 8.0 Hz), 1.68-1.81 (4H, m), 1.59-1.68 (1 H, m). m/z 485.2 (MH⁺).

### Example 90

### (3R)-3-{4-[(4-fluorobenzyl)oxy]phenyl}-N-(1-methyl-1H-tetrazol-5-yl)-1-oxa-8-azaspiro[4.5]decane-8-carboxamide

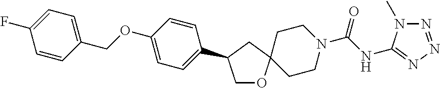

The title compound was prepared from (3R)-3-{4-[(4-fluorobenzyl)oxy]phenyl}-1-oxa-8-azaspiro[4.5]decane hydrochloride (110 mg, 0.322 mmol) as described for Example 84. The crude product was purified by flash chromatography (ethyl acetate(5% ethanol)/heptane) to produce the title compound as a light yellow solid. (49 mg, 0.105 mmol, 33%). ¹H NMR (600 MHz, DMSO-d₆) δ ppm 7.50 (2H, dd, J=8.0, 5.8 Hz), 7.20-7.25 (4H, m), 6.97 (2H, d, J=8.5 Hz), 5.08 (2H, s), 4.12 (1H, t, J=7.8 Hz), 3.82 (2H, s), 3.61-3.71 (3H, m), 3.40-3.53 (3H, m), 2.28 (1H, dd, J=12.4, 8.0 Hz), 1.68-1.82 (6H, m), 1.65 (1H, dd, J=9.5, 3.4 Hz). m/z 467.2 (MH⁺).

### Example 91

### (3S)-3-{4-[(4-fluorobenzyl)oxy]phenyl}-N-(1-methyl-1H-tetrazol-5-yl)-1-oxa-8-azaspiro[4.5]decane-8-carboxamide

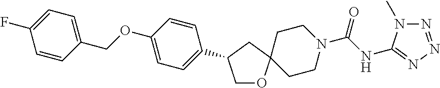

Title compound was prepared from (3S)-3-{4-[(4-fluorobenzyl)oxy]phenyl}-1-oxa-8-azaspiro[4.5]decane hydrochloride (70.0 mg, 0.20 mmol) as described for Example 84. The crude product was purified by flash chromatography (ethyl acetate(5% ethanol)/heptane) to produce the title compound as a light yellow solid. (31 mg, 0.067 mmol, 34%). ¹H NMR (600 MHz, DMSO-d₆) δ ppm 7.50 (2H, dd, J=8.0, 5.8 Hz), 7.20-7.25 (4H, m), 6.97 (2H, d, J=8.5 Hz), 5.08 (2H, s), 4.12 (1H, t, J=7.8 Hz), 3.82 (2H, s), 3.61-3.71 (3H, m), 3.40-3.53 (3H, m), 2.28 (1H, dd, J=12.4, 8.0 Hz), 1.68-1.82 (6H, m), 1.65 (1H, dd, J=9.5, 3.4 Hz). m/z 467.2 (MH⁺).

### 4-nitrophenyl (3RS)-3-[(3-(trifluoromethyl)phenyl]-1-oxa-8-azaspiro[4.5]decane-8-carboxylate

The title compound was prepared from (3RS)-3-[3-(trifluoromethyl)phenyl]-1-oxa-8-azaspiro[4.5]decane hydrochloride (280 mg, 0.87 mmol) as described for 4-nitrophenyl 3-[3-(trifluoromethyl)phenyl]-1-oxa-8-azaspiro[4.5]decane-8-carboxylate Enantiomer 1 to provide title compound as a racemate (380 mg). m/z 467.2 (MH⁺).

### Example 92

### (3RS)—N-(1-methyl-1H-tetrazol-5-yl)-3-[(3-(trifluoromethyl)phenyl]-1-oxa-8-azaspiro[4.5]decane-8-carboxamide

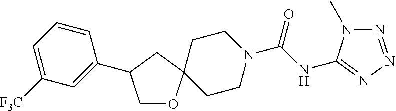

The title compound was prepared from 4-nitrophenyl (3RS)-3-[3-(trifluoromethyl)phenyl]-1-oxa-8-azaspiro[4.5]decane-8-carboxylate (40 mg, 0.089 mmol) as described for Example 93. The crude reaction mixture was concentrated under a stream of nitrogen, dissolved in 1 mL DMSO, and purified by reverse phase HPLC (acetonitrile/water/0.1% formic acid) to give the title compound (12.3 mg). ¹H NMR (400 MHz, DMSO-d₆) δ ppm 1.59-1.71 (m, 2H) 1.73 (d, J=4.39 Hz, 2H) 1.82 (dd, J=12.08, 10.25 Hz, 1H) 2.34 (dd, J=12.63, 7.50 Hz, 1H) 3.43 (dd, J=9.33, 3.48 Hz, 2H) 3.58-3.70 (m, 4H) 3.78 (s, 3H) 4.13-4.20 (m, 1H) 7.53-7.60 (m, 2H) 7.62-7.65 (m, 2H) 9.95 (br. s, 1H). m/z (MH⁺) 411.2.

### 4-nitrophenyl 3-[3-(trifluoromethyl)phenyl]-1-oxa-8-azaspiro[4.5]decane-8-carboxylate Enantiomer 1

A mixture of 3-[3-(trifluoromethyl)phenyl]-1-oxa-8-azaspiro[4.5]decane Enantiomer 1 (197 mg, 0.98 mmol), dioxane (9 mL), and satd sodium bicarbonate (4.5 mL) was slurred. A solution of the 4-nitrophenyl chloroformate (CAS# 7693-46-1) in dioxane (4.5 mL) was added slowly by dropping funnel to the milky white mixture. The reaction was stirred at room temperature for 2 h. The suspension was partitioned between ethyl acetate and ½ satd sodium bicarbonate. The organic extract washed several times with satd sodium bicarbonate and then brine, dried over sodium sulfate, filtered, and concentrated to give the crude product (400 mg, 97%, >90% pure). m/z 451.2 (MH⁺).

### Example 93

### N-(1-methyl-1H-tetrazol-5-yl)-3-[3-(trifluoromethyl)phenyl]-1-oxa-8-azaspiro[4.5]decane-8-carboxamide Enantiomer 1

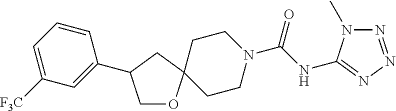

A 0.5 M stock solution of 1-methyl-1H-tetrazole-5-amine sodium salt was prepared by the portion wise addition of sodium hydride (0.63 g, 15.7 mmol) to a solution of 1-methyl-1H-tetrazole-5-amine (CAS# 5422-44-6) (1.49 g, 15 mmol) in DMA (30 mL). The suspension was stirred at RT for 10 min. The suspension was used as is and remainder was kept in refrigerator for later use. 3.6 mL of the 0.5 M 1-methyl-1H-tetrazole-5-amine sodium salt solution (2 eq) was added to 4-nitrophenyl 3-[3-(trifluoromethyl)phenyl]-1-oxa-8-azaspiro[4.5]decane-8-carboxylate Enantiomer 1 (400 mg, 0.888 mmol) in DMA (10 mL) and the mixture was stirred at RT overnight. The reaction was quenched with water, diluted with ethyl acetate and washed with sodium bicarbonate and brine. The crude product stayed in aqueous layer and was, therefore, extracted with 5% EtOH in ethyl acetate (EA). The organic layers were dried over sodium sulfate, filtered, concentrated, and purified by flash chromatography (30 to 100% ethyl acetate/heptane, then 0 to 5% isopropanol/ethyl acetate). The combined fractions were purified by reverse phase HPLC (acetonitrile/water/0.05% TFA). The fractions were isolated, concentrated, redissolved in methanol, and filtered through a StratoSpheres™ PL-HCO₃ MP SPE tube (Polymer Laboratories, Amherst, Mass.) to neutralize any TFA. The filtrate was concentrated to provide title compound (117 mg, 32% yield). ¹H NMR (600 MHz, DMSO-d₆) δ ppm 1.66 (ddd, J=13.33, 9.21, 4.40 Hz, 1H) 1.70-1.78 (m, 3H) 1.85 (dd, J=12.64, 9.89 Hz, 1H) 2.37 (dd, J=12.64, 7.69 Hz, 1H) 3.43-3.50 (m, 2H) 3.68-3.70 (m, 3H) 3.71-3.75 (m, 1H) 3.81 (s, 3H) 4.19-4.21 (m, 1H) 7.56-7.62 (m, 2H) 7.62-7.66 (m, 2H). m/z (MH⁺) calc 411.1756, found 411.1836.

### 4-nitrophenyl 3-[3-(trifluoromethyl)phenyl]-1-oxa-8-azaspiro[4.5]decane-8-carboxylate Enantiomer 2

The title compound was prepared from 3-[3-(trifluoromethyl)phenyl]-1-oxa-8-azaspiro[4.5]decane Enantiomer 2 (197 mg, 0.98 mmol) as described for 4-nitrophenyl 3-[3-(trifluoromethyl)phenyl]-1-oxa-8-azaspiro[4.5]decane-8-carboxylate Enantiomer 1 to provide title compound (410 mg, 97% yield).

### Example 94

### N-(1-methyl-1H-tetrazol-5-yl)-3-[3-(trifluoromethyl)phenyl]-1-oxa-8-azaspiro[4.5]decane-8-carboxamid Enantiomer 2

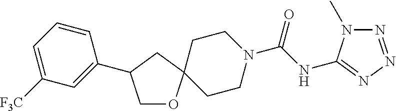

The title compound was prepared from 4-nitrophenyl 3-[3-(trifluoromethyl)phenyl]-1-oxa-8-azaspiro[4.5]decane-8-carboxylate Enantiomer 2 (410 mg, 0.89 mmol) as described for Example 93 to provide the title compound (160 mg, 45% yield). ¹H NMR (400 MHz, DMSO-d₆) δ ppm 1.64-1.68 (m, 1H) 1.72-1.78 (m, 3H) 1.85 (dd, J=12.64, 9.89 Hz, 1H) 2.38 (dd, J=12.64, 7.69 Hz, 1H) 3.43-3.49 (m, 2H) 3.62-3.70 (m, 3H) 3.71-3.75 (m, 1H) 3.81 (s, 3H) 4.20 (t, J=7.42 Hz, 1H) 7.56-7.62 (m, 2H) 7.63-7.67 (m, 2H). m/z (MH⁺) calc 411.1756, found 411.1820.

### 2-(bromomethyl)-5-(trifluoromethyl)pyridine

[5-(trifluoromethyl)pyridin-2-yl]methanol (2.0 g, 11.29 mmol) was dissolved in DCM (20 mL) at r.t. Imidazole (824 mg, 11.9 mmol) was added and allowed to dissolve. Triphenylphosphine (3.36 g, 12.4 mmol) was then added and dissolved. Bromine (0.58 mL, 11.3 mmol) was added last and stirred for 3 hours. The reaction was quenched water and extracted with dichloromethane (2×25 mL). The organics were dried with magnesium sulfate and concentrated. The crude product was purified by flash chromatography (heptanes:ethyl acetate) to give the title compound as a clear liquid (2.1 g, 8.75 mmol, 77%).

### tert-butyl (3R)-3-(4-{[5-(trifluoromethyl)pyridin-2-yl]methoxy}phenyl)-1-oxa-8-azaspiro[4.5]decane-8-carboxylate

tert-butyl (3R)-3-(4-hydroxyphenyl)-1-oxa-8-azaspiro[4.5]decane-8-carboxylate (500 mg, 1.43 mmol) was dissolved in THF (5 mL) and potassium carbonate (257 mg, 1.86 mmol) added. The reaction was stirred at r.t. for 15 minutes and then 2-(bromomethyl)-5-(trifluoromethyl)pyridine (515 mg, 2.15 mmol) added. The reaction was heated at 70 C for 6 hours at which time it was cooled to r.t. quenched with water and the aqueous phase extracted with ethyl acetate (2×10 mL). The organics were dried with magnesium sulfate and concentrated. The crude product was purified by flash chromatography (ethyl acetate heptanes) to give the title compound as a clear oil (590 mg, 1.19 mmol 84%).

### (3R)-3-(4-{[5-(trifluoromethyl)pyridin-2-yl]methoxy}phenyl)-1-oxa-8-azaspiro[4.5]decane

tert-butyl (3R)-3-(4-{[5-(trifluoromethyl)pyridin-2-yl]methoxy}phenyl)-1-oxa-8-azaspiro[4.5]decane-8-carboxylate (590 mg, 1.19 mmol) was dissolved in DCM (3 mL) and TFA (1 mL) added. The reaction was stirred for 1 hour at which time it was quenched with sodium bicarbonate and extracted with drichloromethane. The solution was concentrated to give the title compound as a yellow oil.

### Example 95

### (3R)—N-(1-methyl-1H-tetrazol-5-yl)-3-(4-{[5-(trifluoromethyl)pyridin-2-yl]methoxy}phenyl)-1-oxa-8-azaspiro[4.5]decane-8-carboxamide

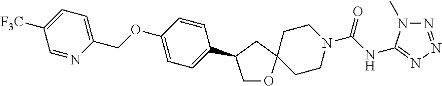

The title compound was prepared from (3R)-3-(4-{[5-(trifluoromethyl)pyridin-2-yl]methoxy}phenyl)-1-oxa-8-azaspiro[4.5]decane (445 mgs, 1.13 mmol) as described for Example 84. Crude product was purified by flash chromatography (ethyl acetate(5% ethanol)/heptane) to produce the title compound as a light yellow solid. (127 mg, 0.246 mmol, 22%). ¹H NMR (400 MHz, DMSO-d₆) δ ppm 9.95 (1H, s), 8.97 (1H, s), 8.24 (1H, dd, J=8.2, 1.8 Hz), 7.71 (1H, d, J=8.2 Hz), 7.22 (2H, d, J=8.6 Hz), 6.97 (2H, d, J=8.6 Hz), 5.27 (2H, s), 4.08 (1 H, t, J=7.7 Hz), 3.78 (3H, s), 3.52-3.69 (3H, m), 2.53 (2H, s), 2.25 (1H, dd, J=12.4, 7.8 Hz), 1.52-1.80 (5 H, m) m/z 518.14 (MH+).

### tert-butyl (3R)-3-(4′-fluorobiphenyl-3-yl)-1-oxa-8-azaspiro[4.5]decane-8-carboxylate

4-Fluorophenyl boronic acid (177 mg, 1.26 mmol) and tert-butyl (3R)-3-(3-{[(trifluoromethyl)sulfonyl]-oxy}phenyl)-1-oxa-8-azaspiro[4.5]decane-8-carboxylate (400 mg, 0.84 mmol) were placed in a flask with 4 mL of toluene and 4 mL of isopropyl alcohol. Cesium carbonate (1.3 g, 5M soln in H₂O, 4.2 mmol) was added and the mixture was degassed with argon. 1,1′-Bis(diphenyl-phosphino)ferrocene palladium dichloride (80 mg, 0.11 mmol) was added and the mixture was heated to 110° C. overnight. The mixture was cooled and diluted with ethyl acetate and filtered. The filtrate was washed with sodium bicarbonate and brine, then dried (MgSO₄), filtered and concentrated. The residue was purified on silica gel chromatography eluting with 20% ethyl acetate/heptane to give the title compound as a yellow oil (300 mg, 86.5%). m/z 434.2 (MNa⁺).

### (3R)-3-(4′-fluorobiphenyl-3-yl)-1-oxa-8-azaspiro[4.5]decane hydrochloride

tert-Butyl (3R)-3-(4′-fluorobiphenyl-3-yl)-1-oxa-8-azaspiro[4.5]decane-8-carboxylate was dissolved in 5 mL of 4N HCl in dioxane and stirred at RT for 3 hours. The solvent was evaporated and the residue was triturated with ether and filtered to give the desired product as an off white solid (300 mg). m/z 312.1 (MH⁺).

### 4-nitrophenyl (3R)-3-(4′-fluorobiphenyl-3-yl)-1-oxa-8-azaspiro[4.5]decane-8-carboxylate

(3R)-3-(4′-Fluorobiphenyl-3-yl)-1-oxa-8-azaspiro[4.5]decane hydrochloride (300 mg, 0.71 mmol) was dissolved in dioxane (10 mL). To this solution was added aqueous saturated sodium bicarbonate (5 mL). After 5 min a solution of para-nitrophenyl chloroformate (153 mg, 0.71 mmol) in dioxane (3 mL) was added dropwise. The bright yellow suspension was stirred at RT for 120 min. The mixture was diluted with ethyl acetate and washed with ½ saturated sodium bicarbonate. The aqueous layer was back extracted once with ethyl acetate and the combined organic layers were washed with brine, then dried (MgSO₄), filtered, and concentrated. The residue was purified on silica gel eluting with 20% ethyl acetate/heptane to give the title compound as a pale yellow oil (300 mg, 88%). m/z 476.8 (MH⁺).

### Example 96

### (3R)-3-(4′-fluorobiphenyl-3-yl)-N-(1-methyl-1H-tetrazol-5-yl)-1-oxa-8-azaspiro[4.5]decane-8-carboxamide

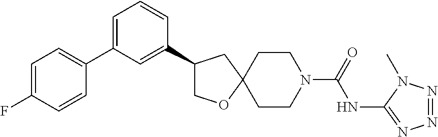

1-Methyl-1H-tetrazol-5-amine (125 mg, 1.26 mmol) was dissolved in anhydrous DMA (2 mL). To this solution was added 60% sodium hydride (50 mg, 1.26 mmol). After stirring at room temperature for 20 min this solution was added to a solution of 4-nitrophenyl (3R)-3-(4′-fluorobiphenyl-3-yl)-1-oxa-8-azaspiro[4.5]decane-8-carboxylate (300 mg, 0.63 mmol) in DMA (4 mL). The mixture was stirred at room temperature under nitrogen for 4 days. The reaction was diluted with ethyl acetate and washed with aqueous saturated sodium bicarbonate. The aqueous layer was back extracted with ethyl acetate. The combined organic layers were washed with brine, dried (MgSO₄), filtered and concentrated. The residue was purified on silica gel eluting with 80% ethyl acetate/heptane increasing to 5% methanol/ethyl acetate. The material was repurified on silica gel eluting with 5% methanol/dichloromethane. The material was dissolved in dichloromethane and washed with brine. The organic layer was dried and concentrated to give the title compound as an orange foamy solid (50 mg, 18%). ¹H NMR (400 MHz, CDCl₃) δ ppm 7.49-7.58 (m, 2H), 7.36-7.45 (m, 3H), 7.21-7.26 (m, 1H), 7.10-7.18 (m, 2H), 4.29 (t, J=8.0 Hz, 1H), 4.04 (d, J=10.9 Hz, 2H), 3.98 (s, 3H), 3.87 (t, J=8.9 Hz, 1H), 3.54-3.69 (m, 2H), 2.32 (dd, J=12.6, 7.9 Hz, 1H), 1.66-2.00 (m, 6H) m/z 437.1 (MH⁺).

### tert-butyl (3R)-3-[4′-(trifluoromethoxy)biphenyl-3-yl]-1-oxa-8-azaspiro[4.5]decane-8-carboxylate

4-Trifluoromethoxy phenyl boronic acid (332 mg, 1.58 mmol) and tert-butyl (3R)-3-(3-{[(trifluoromethyl)sulfonyl]-oxy}phenyl)-1-oxa-8-azaspiro[4.5]decane-8-carboxylate (500 mg, 1.05 mmol) were placed in a flask with 4 mL of toluene and 4 mL of isopropyl alcohol. Cesium carbonate (1.7 g, 5M soln in H₂O, 5.3 mmol) was added and the mixture was degassed with argon. 1,1′-Bis(diphenyl-phosphino)ferrocene palladium dichloride (102 mg, 0.14 mmol) was added and the mixture was heated to 110° C. overnight. The mixture was cooled and diluted with ethyl acetate and filtered. The filtrate was washed with sodium bicarbonate and brine, then dried (MgSO₄), filtered and concentrated. The residue was purified on silica gel chromatography eluting with 20% ethyl acetate/heptane to give the title compound as a yellow oil (360 mg, 70.2%). m/z 500.1 (MH⁺+Na).

### (3R)-3-[4′-(trifluoromethoxy)biphenyl-3-yl]-1-oxa-8-azaspiro[4.5]decane hydrochloride

tert-Butyl (3R)-3-[4′-(trifluoromethoxy)biphenyl-3-yl]-1-oxa-8-azaspiro[4.5]decane-8-carboxylate (360 mg, 0.75 mmol) was dissolved in 5 mL of 4N HCl in dioxane and stirred at room temperature for 1 hour. The solvent was evaporated and the residue was triturated with ether and filtered to give the title compound as an off white solid (350 mg). m/z 377.8 (MH⁺).

### 4-nitrophenyl (3R)-3-[4′-(trifluoromethoxy)biphenyl-3-yl]-1-oxa-8-azaspiro[4.5]decane-8-carboxylate

(3R)-3-[4′-(Trifluoromethoxy)biphenyl-3-yl]-1-oxa-8-azaspiro[4.5]decane hydrochloride (350 mg, 0.75 mmol) was dissolved in dioxane (10 mL). To this solution was added aqueous saturated sodium bicarbonate (5 mL). After 5 min a solution of para-nitrophenyl chloroformate (160 mg, 0.75 mmol) in dioxane (3 mL) was added dropwise. The bright yellow suspension was stirred at room temperature for 2.5 hours. The mixture was diluted with ethyl acetate and washed with ½ saturated sodium bicarbonate. The aqueous layer was back extracted once with ethyl acetate and the combined organic layers were washed with brine. The organic layer was dried (MgSO₄), filtered and concentrated. The residue was purified on silica gel eluting with 20% ethyl acetate/heptane to give the title compound as a clear oil (220 mg, 54.3%). m/z 542.7 (MH⁺).

### Example 97

### (3R)—N-(1-methyl-1H-tetrazol-5-yl)-3-[4′-(trifluoromethoxy)biphenyl-3-yl]-1-oxa-8-azaspiro[4.5]decane-8-carboxamide

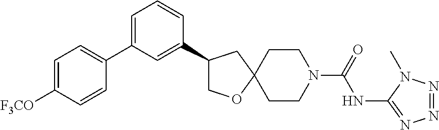

1-Methyl-1H-tetrazol-5-amine (80 mg, 0.81 mmol) was dissolved in anhydrous DMA (2 mL). To this solution was added 60% sodium hydride (20 mg, 0.81 mmol). After stirring at RT for 20 min this solution was added to a solution of 4-nitrophenyl (3R)-3-[4′-(trifluoromethoxy)biphenyl-3-yl]-1-oxa-8-azaspiro[4.5]decane-8-carboxylate (20 mg, 0.41 mmol) in DMA (4 mL). The mixture was stirred at room temperature under nitrogen for 4 days. The reaction was not complete so another 2 equivalents of sodium 1-methyl-1H-tetrazole-5-amine in DMA was added and the reaction was stirred for 3 hours. The reaction was diluted with ethyl acetate and washed with aqueous saturated sodium bicarbonate. The aqueous layer was back extracted with ethyl acetate. The combined organic layers were washed with brine, dried (MgSO₄), filtered and concentrated. The residue was purified on silica gel eluting with 80% ethyl acetate/heptane increasing to 5% methanol/ethyl acetate. The material was repurified on silica gel eluting with 5% methanol/dichloromethane. The purified material was dissolved in dichloromethane and washed with brine to give the title compound as an orange foamy solid (50 mg, 25%). ¹H NMR (400 MHz, CDCl₃) 8 ppm 7.55-7.63 (m, 2H), 7.37-7.45 (m, 3H), 7.24-7.34 (m, 3H), 4.29 (t, J=8.0 Hz, 1H), 4.05 (d, J=11.3 Hz, 2H), 3.98 (s, 3H), 3.87 (t, J=8.9 Hz, 1H), 3.55-3.70 (m, 2H), 2.33 (dd, J=12.6, 8.2 Hz, 1H), 1.67-1.99 (m, 6H). m/z 502.8 (MH⁺).

### tert-butyl (3R)-3-[3-(4,4,5,5-tetramethyl-1,3,2-dioxaborolan-2-yl)phenyl]-1-oxa-8-azaspiro[4.5]decane-8-carboxylate

To tert-butyl (3R)-3-(3-{[(trifluoromethyl)sulfonyl]oxy}phenyl)-1-oxa-8-azaspiro[4.5]decane-8-carboxylate (1.86 g, 4.00 mmol), potassium acetate (1.98 g, 20.0 mmol), bis-(pinacolato)diboron (1.27 g, 5.00 mmol), and 1,1′-bis(diphenylphosphino)ferrocene palladium dichloride (293 mg, 0.40 mmol) was added 1,4 dioxane (15 mL) and the mixture was purged with argon and the vial was capped. The mixture was heated at 100° C. overnight. Heating was continued with good stirring at 100° C. for an additional 4 hrs. The cooled mixture was diluted with diethyl ether (100 mL), filtered reaction through celite and evaporated to give a dark oil. The oil was purified by flash chromatography (5-40% EA/Heptane) to give the title compound as an oil (1.15 g, 65%). m/z 344 (MH⁺ minus Boc).

### tert-butyl (3R)-3-{3-[5-(trifluoromethyl)pyridin-2-yl]phenyl}-1-oxa-8-azaspiro[4.5]decane-8-carboxylate

To tert-butyl (3R)-3-[3-(4,4,5,5-tetramethyl-1,3,2-dioxaborolan-2-yl)phenyl]-1-oxa-8-azaspiro[4.5]decane-8-carboxylate (1.15 g, 2.59 mmol), 2-bromo-5-(trifluoromethyl)pyridine (0.645 g, 2.85 mmol) and sodium carbonate (1.38 g, 13 mmol) suspended in CH₃CN:H₂O (1:1) (16 mL) was added Pd(PPh₃)₂Cl₂ (55 mg, 0.078 mmol). The reaction mixture was heated at 85° C. overnight. After cooling the reaction mixture was extracted with ethyl acetate (2×10 mL), diethyl ether (10 mL), and concentrated in vacuo. The residue was dissolved in THF (˜20 mL) and treated with sodium sulfate and 3-mercaptopropyl silica gel (˜0.3 g, Aldrich, silicycle). This mixture was stirred about 20 min, filtered and the filtrate evaporated. The resulting oil was chromatographed by flash chromatography (5-35% EA/heptane) to give the title compound as an oil (1.04 g, 87%). m/z 463 (MH⁺).

### (3R)-3-{3-[5-(trifluoromethyl)pyridin-2-yl]phenyl}-1-oxa-8-azaspiro[4.5]decane dihydrochloride

To a solution of tert-butyl (3R)-3-{3-[5-(trifluoromethyl)pyridin-2-yl]phenyl}-1-oxa-8-azaspiro[4.5]decane-8-carboxylate (1.04 g, 2.25 mmol) in DCM (6 mL) was added HCl (6 mL, 4M in 1,4-Dioxane, Aldrich). The resulting mixture was allowed to stir for 1 hr and then evaporated to give the title compound as a solid (1.0 g, quant.) m/z 363 (MH⁺).

### Example 98

### (3R)—N-(3,4-dimethylisoxazol-5-yl)-3-{3-[5-(trifluoromethyl)pyridin-2-yl]phenyl}-1-oxa-8-azaspiro[4.5]decane-8-carboxamide

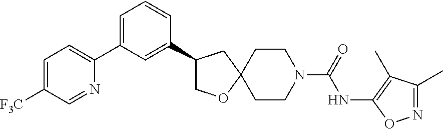

To (3R)-3-{3-[5-(trifluoromethyl)pyridin-2-yl]phenyl}-1-oxa-8-azaspiro[4.5]decane dihydrochloride (260 mg, 0.597 mmol) and phenyl (3,4-dimethylisoxazol-5-yl)carbamate (153 mg, 0.657 mmol) in acetonitrile (5 mL) was added DIEA (0.5 mL, 2.9 mmol) and the reaction was stirred overnight. The mixture was evaporated. The crude material was dissolved in DCM and purified by flash chromatography (50-100% EA/Heptane) to give title compound as a white solid (275 mg, 92%). m/z=501 (MH+). ¹H NMR (400 MHz, DMSO-d₆) δ ppm 1.57-1.73 (m, 4H), 1.74 (s, 3H), 1.87 (dd, J=12.3, 10.6 Hz, 1H), 2.12 (s, 3H), 2.37 (dd, J=12.3, 7.5 Hz, 1H), 3.35-3.47 (m, 2H), 3.54-3.71 (m, 3H), 3.71-3.78 (m, 1H), 4.20 (t, J=7.5 Hz, 1H), 7.44-7.53 (m, 2 H), 7.99-8.04 (m, 1H), 8.09 (s, 1H), 8.20-8.26 (m, 1H), 8.26-8.33 (m, 1H), 9.06 (s, 1H), 9.13 (s, 1H).

### 4-nitrophenyl (3R)-3-{3-[5-(trifluoromethyl)pyridin-2-yl]phenyl}-1-oxa-8-azaspiro[4.5]decane-8-carboxylate

To (3R)-3-{3-[5-(trifluoromethyl)pyridin-2-yl]phenyl}-1-oxa-8-azaspiro[4.5]decane dihydrochloride (0.77 g, 1.77 mmol) was added dioxane (14 mL) and aq satd sodium bicarbonate (7 mL). After about 5 min a solution of para-nitrophenyl chloroformate (357 mg, 1.77 mmol) in dioxane (4 mL) was added dropwise over about 5 min. The bright yellow suspension was stirred at RT for 20 min. The mixture was diluted with ethyl acetate (50 mL) and stood overnight. The organic was washed with saturated sodium bicarbonate (3×50 mL), brine (50 mL), dried (MgSO₄), filtered, and evaporated. The residue was purified by flash chromatography (5-30% EA/heptane) to give the title compound as a foam that solidified upon standing (876 mg, 94%). m/z 528 (MH+).

### Example 99

### (3R)—N-(1-methyl-1H-tetrazol-5-yl)-3-{3-[5-(trifluoromethyl)pyridin-2-yl]phenyl}-1-oxa-8-azaspiro[4.5]decane-8-carboxamide

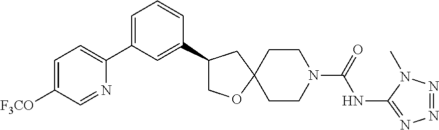

To a solution of 1-methyl-1H-tetrazol-5-amine (227 mg, 2.29 mmol) in anhydrous DMA (4 mL) was added 60% sodium hydride (92 mg, 2.29 mmol). The mixture was stirred at RT for 20 min and then added to a solution of 4-nitrophenyl (3R)-3-{3-[5-(trifluoromethyl)pyridin-2-yl]phenyl}-1-oxa-8-azaspiro[4.5]decane-8-carboxylate (605 mg, 1.15 mmol) in DMA (8 mL). The mixture was stirred at RT under argon for 20 min. The reaction was diluted with ethyl acetate (150 mL) and washed with saturated sodium bicarbonate (1×100 ml and 3×50 mL). The organic layer was washed with brine (50 mL), dried (Na₂SO₄), filtered and concentrated. The residue was purified by reverse phase chromatography (5-95% acetonitrile/water) and the desired fractions were evaporated to a small volume. The aqueous mixture was treated with ethyl acetate (50 mL) and made basic with 0.5N NaOH (50 mL). The organic layer was washed with 0.5 N NaOH (3×50 mL), brine (2×50 mL), dried over magnesium sulfate, filtered and evaporated to give the title compound as a foam (451 mg, 75%). m/z 488 (MH⁺). ¹H NMR (400 MHz, DMSO-d₆) δ ppm 1.47-1.83 (m, 4H), 1.84-1.93 (m, 1H), 2.26-2.42 (m, 1H), 3.38-3.53 (m, 2H), 3.54-3.81 (m, 6H), 4.13-4.25 (m, 1H), 4.13-4.25 (m, 1H), 7.41-7.53 (m, 2H), 7.95-8.12 (m, 2H), 8.17-8.33 (m, 2H), 8.99-9.08 (m, 1H), 9.87 (br. s., 1H).

### Example 100

### (3RS)-3-(3-chloro-5-fluorophenyl)-N-(3,4-dimethylisoxazol-5-yl)-3-fluoro-1-oxa-8-azaspiro[4.5]decane-8-carboxamide

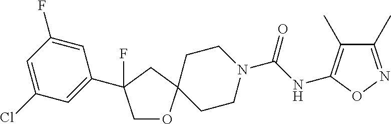

To a crude solution of tert-butyl (3RS)-3-(3-chloro-5-fluorophenyl)-3-hydroxy-1-oxa-8-azaspiro[4.5]decane-8-carboxylate (prepared as described in the synthesis of tert-butyl (3RS)-3-(3-chloro-5-fluorophenyl)-1-oxa-8-azaspiro[4.5]decane-8-carboxylate; 285 mg, 0.739 mmol) was added DAST (0.05 mL, 0.4 mmol) at −78 C and stirred for 2 hr. Another portion of DAST (0.1 mL, 0.8 mmol) was added at −78 and the reaction was warmed to RT overnight. The reaction was quenched with water and layers separated then extracted with EA (2×5 mL). The organic layer was dried over MgSO4, filtered, and concentrated to give 250 mg of a crude oil. To the oil in DCM (2.0 mL) was added 4N HCl/dioxane (1.0 mL). The reaction mixture was stirred for 2 hrs and then evaporated. To the residue (207 mg, 0.638 mmol) and phenyl (3,4-dimethylisoxazol-5-yl)carbamate (150 mg, 0.65 mmol) was added acetonitrile (4 mL), DIEA (0.5 mL, 3.0 mmol) and the reaction was stirred overnight. The mixture was treated with TFA (0.25 mL) and purified by reverse phase chromatography (5-95% acetonitrile/water/0.05% TFA) to give an oil. The oil was dissolved in 1 mL DMSO, and purified by reverse phase HPLC (acetonitrile/water/0.1°)/0 formic acid) to give the title compound (26 mg, 8%). m/z 426.14 (MH⁺). ¹H NMR (400 MHz, DMSO-d₆) δ ppm 9.15 (s, 1H), 7.47-7.51 (m, 1H), 7.46 (s, 1H), 7.37-7.42 (m, 1H), 4.16-4.27 (m, 1H), 3.99-4.12 (m, 1H), 3.54-3.65 (m, 2H), 3.37-3.46 (m, 2H), 2.29-2.46 (m, 2H), 2.12 (s, 3H), 1.74 (s, 3H), 1.67-1.84 (m, 4H).

### Phenyl 1,2-benzisoxazol-3-ylcarbamate

A solution of 1,2-benzisoxazol-3-amine (1.00 g; CAS# 36216-80-5) and triethylamine (1.09 mL) in acetonitrile (5 mL) was added dropwise to at 0° C. solution of phenyl chloroformate (0.989 mL) in THF (20 mL). The reaction was stirred at 0° C. for 1 h and then allowed to warm to RT overnight. The reaction was diluted with ethyl acetate and washed with 1N HCl and brine. The organic layer was dried over sodium sulfate, filtered, and concentrated to give the crude product as a reddish brown solid. The solid was triturated with refluxing diisopropyl ether, cooled to RT, and filtered to give final product as a tan solid (1.22 g, 64%). m/z 255 (MH⁺).

### Example 101

### (3RS)—N-1,2-benzisoxazol-3-yl-3-{3-[(5-chloropyridin-2-yl)oxy]phenyl}-1-oxa-8-azaspiro[4.5]decane-8-carboxamide

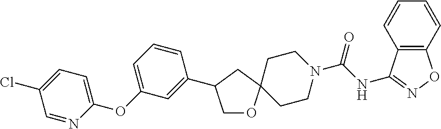

The title compound was prepared from (3RS)-3-{3-[(5-chloropyridin-2-yl)oxy]phenyl}-1-oxa-8-azaspiro[4.5]decane hydrochloride (prepared from the racemate in a manner similar to that described for (3R)-3-{3-[(5-chloropyridin-2-yl)oxy]phenyl}-1-oxa-8-azaspiro[4.5]decane hydrochloride) and phenyl 1,2-benzisoxazol-3-ylcarbanmnate using same procedure as described for the preparation of Example 13, Step 3 to give the racemic title compound (18.6 mg). LCMS t_{R}=2.16 min (Phenomenex Gemini C18 4.6×50 mm 5 μm; 0.04% Formic Acid, 0.01% TFA/MeCN), m/z 505.45 (MH⁺).

### Preparation of Enantiomerically-Enriched tert-Butyl 3-[3-(benzyloxy)phenyl]-1-oxa-8-azaspiro[4.5]decane-8-carboxylate

To a pressure reaction vessel was added tert-butyl (3RS)-3-[3-(benzyloxy)phenyl]-1-oxa-8-azaspiro[4.5]dec-3-ene-8-carboxylate (100 mg) and a catalyst ([Rh(COD)((R)-TCFP)]⁻BF₄⁺ or [Rh(COD)((S)-TCFP)]⁻BF₄⁺; 2 mol %) which were purged with nitrogen 10 times. Via a manual valve, nitrogen sparged trifluoroethanol (2 mL) was added to the solids. The reaction mixture was purged with nitrogen and then purged with hydrogen using the pressurize-then-vent method of purging. The reaction mixture was heated to 70° C. and then pressurized to 200 psig with hydrogen After 48 h at 70° C. and 200 psig of hydrogen, the reaction was cooled to 30° C. and purged with nitrogen. The reactions were analyzed by chiral SFC (4.6 mm×250 mm Chiralpak AD-H, 5% to 45% methanol/CO₂/0.1°)/0 diethylamine at 4 ml/min, 40° C.): For catalyst [Rh(COD)((R)-TCFP)]⁻BF₄⁺ (CAS#705945-70-; Hoge et al., J. Am. Chem. Soc. 2004, 126, 5966-5967), a 100% conversion to enantiomerically enriched tert-butyl 3-[3-(benzyloxy)phenyl]-1-oxa-8-azaspiro[4.5]decane-8-carboxylate was observed (96% ee of the first eluting enantiomer, t_{R}=5.7 min). For catalyst [Rh(COD)((S)-TCFP)]⁻BF₄⁺ (CAS#705945-68-2; Hoge et al., J. Am. Chem. Soc. 2004, 126, 5966-5967), a 100% conversion to the other enantiomer of tert-butyl 3-[3-(benzyloxy)phenyl]-1-oxa-8-azaspiro[4.5]decane-8-carboxylate was observed (96% ee of the second eluting enantiomer, t_{R}=6.3 min). For catalyst (5)-1-[(R)-2-di-(4-fluorophenyl)phosphino)ferrocenyl]ethyldi-tert-butylphosphinerhodium(I)cyclooctadiene trifluoromethanesulfonate (Solvias AG, Basel, Switzerland, #SJ-J014-2B), a 100% conversion to tert-butyl 3-[3-(benzyloxy)phenyl]-1-oxa-8-azaspiro[4.5]decane-8-carboxylate with approximately 100% ee was observed.

The biological activities of compounds described in the above examples were determined using the following assays.

### FAAH Assay

The FAAH assay was carried out in 384-well clear polystyrene plates (Evergreen Scientific) in a total volume of 50 μl per well in a manner similar to that described by Mileni et al., Proc. Nat. Acad. Sci. 2008, 105, 12820-12824. All percents are by volume. Serial dilutions of compound were initially prepared in 100% DMSO, and then diluted two-fold into HPLC-grade H₂O to give 50% DMSO. To each well, was placed the reaction mixture (40 μl) containing 1-4 nM FAAH, 50 mM NaPᵢ, pH 7.4, 3 mM α-ketoglutarate, 0.15 mM NADH, 7.5 U/ml glutamate dehydrogenase, 2 mM ADP, 1 mM EDTA, and 0.1% Triton X-100 (The concentration shown for each component is the final concentration in the assay). To this mixture, was added 5 μl of a compound of Examples 1 to 101 at various concentrations in 50% DMSO (or 5 μl 50% DMSO for controls). This was immediately followed by the addition of 5 μl oleamide (500 μM) dissolved in 75% EtOH/25% DMSO and the reaction mixture was mixed for 1.5 min. The final concentrations of DMSO and EtOH in the assay were each 7.5%. The reactions were incubated at 30° C. and the absorbance at 340 nm was collected over a period of 90 min with readings taken in 30-second intervals using SpectraMax Plus³⁸⁴ Microplate Spectrophotometer (Molecular Devices, Sunnyvale, Calif.). The human FAAH and rat FAAH used in the assay was prepared as described in the patent application WO 2006/067613 using wild-type E. coli cells transformed in St. Louis, Mo., U.S.A. The purity of the enzyme was greater than 98% based on an analysis by SDS-polyacrylamide gel electrophoresis followed by Coomassie Blue staining.

### Kinetic Data Analyses

Reaction progress curves were corrected for the non-enzymatic oxidation of NADH by subtraction of absorbancies at each time point obtained from control reactions containing no FAAH enzyme. The loss of enzyme activity as a function of time is well-described by the following mathematical equation (1) for a mono-exponential decay:

Aₜ=A₀+C*e^{(-kobs*t)} (1)

where Aₜ represents the absorbance at time t, A₀ represents the absorbance at time zero, and C represents a constant. Observed rates of enzyme inactivation (k_{obs}) were determined from the non-linear reaction progress curves by fitting the corrected absorbancies to an equation for a mono-exponential decay using the third-party Microsoft Excel plug-in, XLfit (IDBS Limited). Secondary plots of k_{obs} vs. inhibitor concentration were prepared from k_{obs} values obtained from progress curves. Second-order rates for enzyme inactivation, expressed as k_{inact}/Kᵢ (M⁻¹ s⁻¹), were calculated from the slopes from linear regression analysis of the secondary plot of k_{obs} vs. inhibitor concentration as defined in following equation (2), where [I]<<Kᵢ:

$$Slope = \frac{{k}_{obs}}{[ I ]} = \frac{{k}_{inact}}{{K}_{i} * ( 1 + [ S ] / {K}_{m} )} \qquad ( 2 )$$

The concentration of substrate in the assay was equal to the Kₘ for oleamide of 50 μM. Therefore, reported k_{inact}/Kᵢ values are obtained by multiplying resulting slopes by a factor of two (i.e. slope=k_{inact}/(Kᵢ*2)).

Table 1, below, lists human FAAH (hFAAH) and rat FAAH (rFAAH) enzyme inhibition values for Examples 1-101 as a ratio of k_{inact}/Kᵢ (M⁻¹ s⁻¹).

TABLE 1
In vitro hFAAH and rFAAH k_{inact}/Kᵢ
(M⁻¹s⁻¹) Values for Examples 1-101.

| | hFAAH | rFAAH |
|---|---|---|
| | k_{inact}/Kᵢ | k_{inact}/Kᵢ |
| Ex. | (M⁻¹s⁻¹) | (M⁻¹s⁻¹) |
| 1 | 3130 | 1880 |
| 2 | 1340 | 1130 |
| 3 | 3040 | 2240 |
| 4 | 2910 | 3380 |
| 5 | 2660 | 3620 |
| 6 | 2320 | 1750 |
| 7 | 2890 | 526 |
| 8 | 1950 | 3520 |
| 9 | 4970 | 3340 |
| 10 | 2690 | 5890 |
| 11 | 2420 | 1020 |
| 12 | 2190 | 2270 |
| 13 | 1800 | 3110 |
| 14 | 2040 | 2670 |
| 15 | 2430 | 4360 |
| 16 | 1990 | 3590 |
| 17 | 6960 | 6690 |
| 18 | 2290 | 2840 |
| 19 | 6710 | 7120 |
| 20 | 3970 | 4390 |
| 21 | 3100 | 3550 |
| 22 | 6030 | 3630 |
| 23 | 6950 | 10900 |
| 24 | 788 | 431 |
| 25 | 2090 | 2240 |
| 26 | 2140 | 2520 |
| 27 | 2090 | 1140 |
| 28 | 1580 | 3950 |
| 29 | 2110 | 3230 |
| 30 | 7380 | 5800 |
| 31 | 3580 | 3220 |
| 32 | 4120 | 7580 |
| 33 | 3420 | 4070 |
| 34 | 1840 | 2610 |
| 35 | 2390 | 1560 |
| 36 | 2590 | 3830 |
| 37 | 4130 | 4540 |
| 38 | 2050 | 2380 |
| 39 | 1810 | 1510 |
| 40 | 2470 | 3740 |
| 41 | 12500 | 15800 |
| 42 | 5590 | 5320 |
| 43 | 3620 | 5870 |
| 44 | 1330 | 1950 |
| 45 | 6240 | 7130 |
| 46 | 4490 | 6920 |
| 47 | 9350 | 6260 |
| 48 | 4470 | 2540 |
| 49 | 16800 | 21900 |
| 50 | 7470 | 9110 |
| 51 | 16700 | 17300 |
| 52 | 10100 | 6170 |
| 53 | 2020 | 1470 |
| 54 | 2040 | 1110 |
| 55 | 2070 | 1160 |
| 56 | 1880 | 1140 |
| 57 | 1610 | 2110 |
| 58 | 2780 | 707 |
| 59 | 3130 | 879 |
| 60 | 2110 | 2750 |
| 61 | 1220 | 433 |
| 62 | 2920 | 2260 |
| 63 | 2350 | 2160 |
| 64 | 1490 | 588 |
| 65 | 1100 | 2630 |
| 66 | 2140 | 2920 |
| 67 | 1330 | 2680 |
| 68 | 1920 | 3030 |
| 69 | 960 | 4880 |
| 70 | 2110 | 2390 |
| 71 | 2610 | 1020 |
| 72 | 4250 | 1500 |
| 73 | 2150 | 1340 |
| 74 | 2380 | 1140 |
| 75 | 3570 | 1610 |
| 76 | 3120 | 1150 |
| 77 | 7110 | 4560 |
| 78 | 7100 | 3840 |
| 79 | 5600 | 1970 |
| 80 | 11400 | 5520 |
| 81 | 4890 | 11200 |
| 82 | 5110 | 1500 |
| 83 | 5480 | 974 |
| 84 | 8420 | 1500 |
| 85 | 5980 | 1340 |
| 86 | 28400 | 11200 |
| 87 | 15900 | 4110 |
| 88 | 32600 | 25000 |
| 89 | 29800 | 9640 |
| 90 | 30800 | 22600 |
| 91 | 21200 | 7500 |
| 92 | 5240 | 3120 |
| 93 | 4600 | 2160 |
| 94 | 3760 | 2110 |
| 95 | 21500 | 30300 |
| 96 | 26300 | 2010 |
| 97 | 16900 | 1830 |
| 98 | 2820 | 1630 |
| 99 | 9970 | 2690 |
| 100 | 871 | 1120 |
| 101 | 1780 | 8330 |

### In Vivo Complete Freund's Adjuvant (CFA) Efficacy Assay

For additional information on the CFA efficacy assay, see Jayamanne et al., Brit. J. Pharmacol. 2006, 147, 281-288. Experiments were performed on adult Male Sprague-Dawley Rats (200 g-250 g). Inflammation was induced in the left hindpaw of the rat by an intra-plantar injection of 150 uL Complete Freund's Adjuvant (CFA) (SIGMA F5881). The CFA injection immediately induces local inflammation, paw swelling, and pain that persists for at least two weeks post-injection. Baseline paw withdrawal threshold (PWT) was measured to determine the percent inhibition of allodynia using a set of Von Frey Hairs on day 4 post injection as illustrated by the Dixon Up and Down Method (W. J. Dixon, Ann. Rev. Pharmacol. Toxicol. 1980, 20:441-462). Animals that exhibit the pain criteria of 9 grams or less were then placed on study. Test compound was administered at a concentration of 3 mg/kg (mpk) orally with the dosing vehicle 5% N,N′-Dimethylacetamide (SIGMA D137510) and 95% (40% 2-hydroxypropyl-beta-cyclodextrin in water) (SIGMA H107). Following Dose administration PWT threshold was evaluated again at four hours postdose. The Sprague-Dawley rats used in this assay were purchased from Harlan, 8520 Allison Pointe Blvd., Indianapolis, Ind., 46250, U.S.A. Sprague-Dawley rats are an outbred breed of albino rats first produced by the Sprague Dawley farms in Madison, Wis., U.S.A.

### Data Analysis

Percent Inhibition of Allodynia was determined by the formula:

$$\% Inhibition of Allodynia = 100 \times \frac{Δ PWT ( test compound ) \text{-} mean Δ PWT ( vehicle )}{Baseline \text{-} mean Δ PWT ( vehicle )}$$

ΔPWT measurements were averaged for each treatment group and statistical comparisons between groups were made using ANOVA and Dunnett's two-tailed test. Test compounds that increased percent inhibition significantly when compared to the vehicle group (p<0.05 ANOVA/Dunnetts) were determined to be efficacious. Table 2, below, lists CFA efficacy for examples assayed.

TABLE 2
in vivo CFA Efficacy for examples tested at 3 mpk, oral dosing.

| | CFA |
|---|---|
| Ex. | Efficacy^{a,b} |
| 1 | (+)^{d} |
| 2 | (−)^{c} |
| 3 | (−)^{c} |
| 7 | (+) |
| 8 | (+) |
| 9 | (+) |
| 10 | (+) |
| 11 | (+) |
| 12 | (+) |
| 33 | (+) |
| 34 | (+) |
| 35 | (+) |
| 36 | (+) |
| 37 | (+) |
| 39 | (+) |
| 41 | (+) |
| 42 | (+) |
| 43 | (+) |
| 44 | (+) |
| 45 | (+) |
| 46 | (+) |
| 47 | (+) |
| 48 | (+) |
| 49 | (−) |
| 50 | (−) |
| 51 | (+) |
| 52 | (+) |
| 57 | (+) |
| 59 | (+) |
| 60 | (+) |
| 61 | (+) |
| 62 | (+) |
| 63 | (+) |
| 68 | (+) |
| 72 | (+) |
| 73 | (+) |
| 75 | (+) |
| 76 | (+) |
| 77 | (+) |
| 78 | (+) |
| 79 | (+) |
| 80 | (+) |
| 81 | (+) |
| 82 | (+) |
| 83 | (+) |
| 84 | (+) |
| 85 | (+) |
| 87 | (+) |
| 88 | (+) |
| 89 | (+) |
| 90 | (+) |
| 91 | (+) |
| 93 | (+) |
| 94 | (+) |
| 95 | (+) |
| 96 | (+) |
| 97 | (+) |
| 99 | (+) |

^{a}Test compounds that increased percent inhibition of allodynia significantly when compared to the vehicle group (p < 0.05 ANOVA/Dunnetts) were determined to be efficacious; (+) indicates the test compound was determined to be efficacious at 3 mpk, oral dosing; (−) indicates the test compound was determined to not be efficacious at 3 mpk, oral dosing.
^{b}some compounds were tested at different doses as indicated below.
^{c}compound tested at 10 mpk, intraperitoneal dosing.
^{d}compound tested at 25 mpk, intraperitoneal dosing.

1. A compound of Formula I: 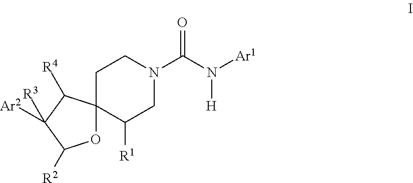
wherein:
Ar¹ is selected from: 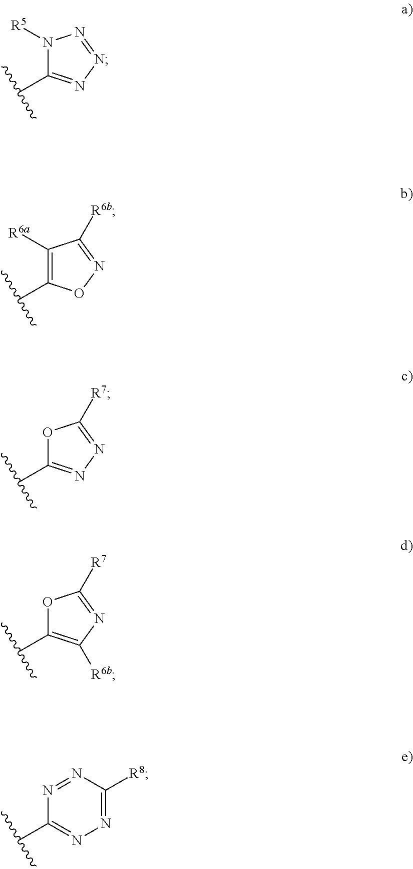
f) benzoisoxazole optionally substituted by 1 to 3 halo, C₁-C₃ alkyl, C₁-C₃ alkoxy, C₁-C₃ haloalkyl or C₁-C₃ haloalkoxy substituents; or
g) pyridine, pyridazine, pyrimidine, or pyrazine; wherein the pyridine, pyridazine, pyrimidine, or pyrazine is optionally substituted by 1 to 3 halo, C₁-C₃ alkyl, —(CH₂)ₙ—(C₃-C₆ cycloalkyl), C₁-C₃ alkoxy, C₁-C₃ haloalkyl or C₁-C₃ haloalkoxy substituents;
Ar² is selected from:
a) phenyl optionally substituted by 1 to 5 halo, C₁-C₆ alkyl, —(CH₂)ₙ—(C₃-C₆ cycloalkyl), C₁-C₆ alkoxy, —(CH₂)ₙ—(C₃-C₆ cycloalkoxy), C₁-C₆ haloalkyl, C₁-C₆ haloalkoxy, —O—CH₂—CH₂—O—(C₁-C₆ alkyl), or —O—CH₂—CH₂—O—(C₁-C₆ haloalkyl) substituents; wherein the phenyl is further optionally substituted by a substituent of the formulae —R⁹, —O—R⁹, —O—(CH₂)ₚ—R⁹, or —(CH₂)ₚ—O—R⁹;
b) oxazole, isoxazole, thiazole, isothiazole, oxadiazole, or thiadiazole substituted by a substituent of the formulae —(CH₂)ₙ—R⁹, —(CH₂)ₘ—O—R⁹, or —(CH₂)ₚ—O—(CH₂)ₚ—R⁹;
c) a heterocycle of the formula: 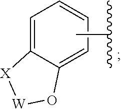
wherein X is CH₂ or O, and W is (CH₂)ₘ or CF₂; or
d) naphthyl, quinolinyl or isoquinolinyl optionally substituted by 1 to 3 halo, C₁-C₃ alkyl, C₁-C₃ alkoxy, C₁-C₃ haloalkyl or C₁-C₃ haloalkoxy substituents;
R¹ is hydrogen, F, or CH₃;
R² is hydrogen or CH₃;
R³ is hydrogen, CH₃, —O—CH₃, OH, CN, or F;
R⁴ is hydrogen, F, or CH₃;
R⁵ is hydrogen, C₁-C₆ alkyl, —(CH₂)ₙ—(C₃-C₆ cycloalkyl), or C₁-C₆ haloalkyl;
R^{ha} is C₁-C₃ alkyl;
R^{6b} is hydrogen, C₁-C₆ alkyl, or C₁-C₃ haloalkyl;
R⁷ is C₁-C₃ alkyl, —(CH₂)ₙ—(C₃-C₆ cycloalkyl), R⁹, or —CH₂—O—R⁹;
R⁸ is phenyl optionally substituted by from 1 to 3 halo, C₁-C₃ alkyl, C₁-C₃ alkoxy, C₁-C₃ haloalkyl or C₁-C₃ haloalkoxy substituents;
R⁹ is selected from phenyl, naphthyl, or heteroaryl; wherein R⁹ is optionally substituted by from 1 to 3 halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, or C₁-C₃ haloalkoxy substituents;
m is 1, 2 or 3; n is 0, 1, 2, 3 or 4; and p is 1 or 2;
or a pharmaceutically acceptable salt thereof.

2. A compound of Formula I according to claim 1 wherein:
Ar¹ is selected from: 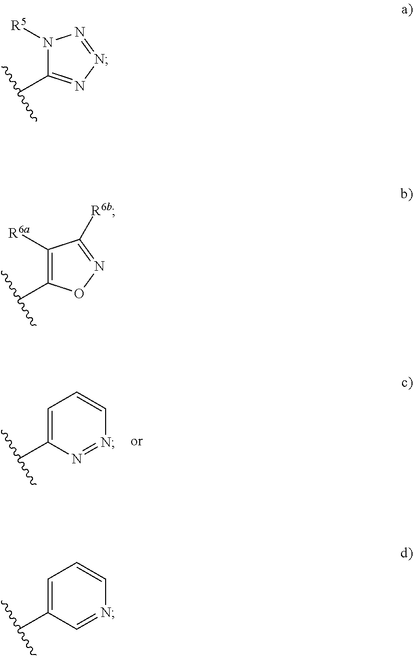
Ar² is selected from:
a) a phenyl ring optionally substituted by from 1 to 3 halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, C₁-C₃ haloalkoxy groups; wherein the phenyl ring may also be substituted by a group of the formulae —R⁹, —O—R⁹, —O—CH₂—R⁹, or —O—(CH₂)₂—O—R⁹; or
b) a thiazole or oxadiazole ring substituted by a group of the formulae —R⁹;
R¹, R², and R⁴ are hydrogen;
R³ is hydrogen or F;
R⁵ is C₁-C₃ alkyl;
R^{6a} is methyl;
R^{6b} is hydrogen or C₁-C₃ alkyl;
R⁹ is phenyl, pyridine, or pyrimidine; wherein the R⁹ ring is optionally substituted by from 1 to 3 groups selected from halo, C₁-C₃ alkyl, —(CH₂), —(C₃-C₆ cycloalkyl), C₁-C₃ alkoxy, —(CH₂)ₙ—(C₃-C₆ cycloalkoxy), C₁-C₃ haloalkyl or C₁-C₃ haloalkoxy groups;
or a pharmaceutically acceptable salt thereof.

3. A compound of Formula I according to claim 2 wherein:
Ar² is selected from:
a) a phenyl ring optionally substituted by from 1 to 3 groups selected from F, Cl, Br, methyl, ethyl, CF₃, OCH₃, or OCF₃; wherein the phenyl ring may also be substituted by a group of the formulae —R⁹, —O—R⁹ or —O—CH₂—R⁹;
b) a thiazole or oxadiazole ring substituted by a group of the formulae —R⁹;
R⁵, R^{6a}, and R^{6b} are methyl;
R⁹ is phenyl, pyridine, or pyrimidine; wherein the R⁹ ring is optionally substituted by from 1 to 3 substituents selected from F, Cl, Br, CF₃, or OCF₃;
or a pharmaceutically acceptable salt thereof.

4. A compound of claim 1 selected from the group of:
N-pyridazin-3-yl-3-(3-{[5-(trifluoromethyl)pyridin-2-yl]oxy}phenyl)-1-oxa-8-azaspiro[4.5]decane-8-carboxamide;
3-{3-[(5-chloropyridin-2-yl)oxy]phenyl}-N-pyridazin-3-yl-1-oxa-8-azaspiro[4.5]decane-8-carboxamide;
3-{3-[(5-bromopyridin-2-yl)oxy]phenyl}-N-pyridazin-3-yl-1-oxa-8-azaspiro[4.5]decane-8-carboxamide;
3-{3-[(5-bromopyridin-2-yl)oxy]phenyl}-N-pyridin-3-yl-1-oxa-8-azaspiro[4.5]decane-8-carboxamide;
N-(3,4-dimethylisoxazol-5-yl)-3-[3-(trifluoromethoxy)phenyl]-1-oxa-8-azaspiro[4.5]decane-8-carboxamide;
3-(3-chlorophenyl)-N-(3,4-dimethylisoxazol-5-yl)-1-oxa-8-azaspiro[4.5]decane-8-carboxamide;
3-(3,4-dichlorophenyl)-N-(3,4-dimethylisoxazol-5-yl)-1-oxa-8-azaspiro[4.5]decane-8-carboxamide;
3-(3-chloro-5-fluorophenyl)-N-(3,4-dimethylisoxazol-5-yl)-1-oxa-8-azaspiro[4.5]decane-8-carboxamide;
N-(3,4-dimethylisoxazol-5-yl)-3-{4-[(4-fluorobenzyl)oxy]phenyl}-1-oxa-8-azaspiro[4.5]decane-8-carboxamide;
3-{3-chloro-4-[(2-chlorobenzyl)oxy]phenyl}-N-(3,4-dimethylisoxazol-5-yl)-1-oxa-8-azaspiro[4.5]decane-8-carboxamide;
3-{3-chloro-4-[(4-fluorobenzyl)oxy]phenyl}-N-(3,4-dimethylisoxazol-5-yl)-1-oxa-8-azaspiro[4.5]decane-8-carboxamide;
3-[4-(benzyloxy)-3-chlorophenyl]-N-(3,4-dimethylisoxazol-5-yl)-1-oxa-8-azaspiro[4.5]decane-8-carboxamide;
3-{3-chloro-4-[(3-chlorobenzyl)oxy]phenyl}-N-(3,4-dimethylisoxazol-5-yl)-1-oxa-8-azaspiro[4.5]decane-8-carboxamide;
N-(3,4-dimethylisoxazol-5-yl)-3-{4-[(3-fluorobenzyl)oxy]phenyl}-1-oxa-8-azaspiro[4.5]decane-8-carboxamide;
3-{4-[(4-chlorobenzyl)oxy]phenyl}-N-(3,4-dimethylisoxazol-5-yl)-1-oxa-8-azaspiro[4.5]decane-8-carboxamide;
3-(1,3-benzodioxol-5-yl)-N-(3,4-dimethylisoxazol-5-yl)-1-oxa-8-azaspiro[4.5]decane-8-carboxamide;
N-(3,4-dimethylisoxazol-5-yl)-3-(2-naphthyl)-1-oxa-8-azaspiro[4.5]decane-8-carboxamide;
3-(4-chloro-3-fluorophenyl)-N-(3,4-dimethylisoxazol-5-yl)-1-oxa-8-azaspiro[4.5]decane-8-carboxamide;
3-(4-chloro-2-fluorophenyl)-N-(3,4-dimethylisoxazol-5-yl)-1-oxa-8-azaspiro[4.5]decane-8-carboxamide;
3-(3-chloro-4-fluorophenyl)-N-(3,4-dimethylisoxazol-5-yl)-1-oxa-8-azaspiro[4.5]decane-8-carboxamide;
N-(3,4-dimethylisoxazol-5-yl)-3-(4-{[6-(trifluoromethyl)pyridin-2-yl]methoxy}phenyl)-1-oxa-8-azaspiro[4.5]decane-8-carboxamide;
3-{4-[(3,4-difluorobenzyl)oxy]phenyl}-N-(3,4-dimethylisoxazol-5-yl)-1-oxa-8-azaspiro[4.5]decane-8-carboxamide;
3-{4-[(2,5-difluorobenzyl)oxy]phenyl}-N-(3,4-dimethylisoxazol-5-yl)-1-oxa-8-azaspiro[4.5]decane-8-carboxamide;
N-(3,4-dimethylisoxazol-5-yl)-3-(4-{[5-(trifluoromethyl)pyridin-2-yl]methoxy}phenyl)-1-oxa-8-azaspiro[4.5]decane-8-carboxamide;
3-(3′,4′-difluorobiphenyl-3-yl)-N-(3,4-dimethylisoxazol-5-yl)-1-oxa-8-azaspiro[4.5]decane-8-carboxamide;
N-(3,4-dimethylisoxazol-5-yl)-3-(4′-fluorobiphenyl-3-yl)-1-oxa-8-azaspiro[4.5]decane-8-carboxamide;
3-(4′-chlorobiphenyl-3-yl)-N-(3,4-dimethylisoxazol-5-yl)-1-oxa-8-azaspiro[4.5]decane-8-carboxamide;
N-(3,4-dimethylisoxazol-5-yl)-3-[4′-(trifluoromethoxy)biphenyl-3-yl]-1-oxa-8-azaspiro[4.5]decane-8-carboxamide;
N-(3,4-dimethylisoxazol-5-yl)-3-[3-(trifluoromethyl)phenyl]-1-oxa-8-azaspiro[4.5]decane-8-carboxamide;
3-(3,4-dichlorophenyl)-N-(3-ethyl-4-methylisoxazol-5-yl)-1-oxa-8-azaspiro[4.5]decane-8-carboxamide;
3-(3,4-dichlorophenyl)-N-(4-methyl-3-propylisoxazol-5-yl)-1-oxa-8-azaspiro[4.5]decane-8-carboxamide;
N-(5-methyl-1,3,4-oxadiazol-2-yl)-3-[3-(trifluoromethoxy)phenyl]-1-oxa-8-azaspiro[4.5]decane-8-carboxamide;
N-(3,4-dimethylisoxazol-5-yl)-3-{5-[4-(trifluoromethoxy)phenyl]-1,2,4-oxadiazol-3-yl}-1-oxa-8-azaspiro[4.5]decane-8-carboxamide;
N-(3,4-dimethylisoxazol-5-yl)-3-{5-[4-(trifluoromethyl)phenyl]-1,2,4-oxadiazol-3-yl}-1-oxa-8-azaspiro[4.5]decane-8-carboxamide;
N-(1-methyl-1H-tetrazol-5-yl)-3-[3-(trifluoromethoxy)phenyl]-1-oxa-8-azaspiro[4.5]decane-8-carboxamide;
N-(1-methyl-1H-tetrazol-5-yl)-3-[3-(trifluoromethoxy)phenyl]-1-oxa-8-azaspiro[4.5]decane-8-carboxamide, (enantiomer 1);
N-(1-methyl-1H-tetrazol-5-yl)-3-[3-(trifluoromethoxy)phenyl]-1-oxa-8-azaspiro[4.5]decane-8-carboxamide, (enantiomer 2);
3-(3-chlorophenyl)-N-(1-methyl-1H-tetrazol-5-yl)-1-oxa-8-azaspiro[4.5]decane-8-carboxamide;
3-{3-[(5-chloropyridin-2-yl)oxy]phenyl}-N-(1-methyl-1H-tetrazol-5-yl)-1-oxa-8-azaspiro[4.5]decane-8-carboxamide;
N-(1-methyl-1H-tetrazol-5-yl)-3-{5-[4-(trifluoromethyl)phenyl]-1,2,4-oxadiazol-3-yl}-1-oxa-8-azaspiro[4.5]decane-8-carboxamide;
N-(1-methyl-1H-tetrazol-5-yl)-3-{5-[4-(trifluoromethoxy)phenyl]-1,2,4-oxadiazol-3-yl}-1-oxa-8-azaspiro[4.5]decane-8-carboxamide;
3-{4-[(3-fluorobenzyl)oxy]phenyl}-N-(1-methyl-1H-tetrazol-5-yl)-1-oxa-8-azaspiro[4.5]decane-8-carboxamide;
3-{4-[(3,4-difluorobenzyl)oxy]phenyl}-N-(1-methyl-1H-tetrazol-5-yl)-1-oxa-8-azaspiro[4.5]decane-8-carboxamide;
3-{4-[(4-fluorobenzyl)oxy]phenyl}-N-(1-methyl-1H-tetrazol-5-yl)-1-oxa-8-azaspiro[4.5]decane-8-carboxamide;
N-(1-methyl-1H-tetrazol-5-yl)-3-[3-(trifluoromethyl)phenyl]-1-oxa-8-azaspiro[4.5]decane-8-carboxamide;
N-(1-methyl-1H-tetrazol-5-yl)-3-(4-{[5-(trifluoromethyl)pyridin-2-yl]methoxy}phenyl)-1-oxa-8-azaspiro[4.5]decane-8-carboxamide;
3-(4′-fluorobiphenyl-3-yl)-N-(1-methyl-1H-tetrazol-5-yl)-1-oxa-8-azaspiro[4.5]decane-8-carboxamide;
N-(1-methyl-1H-tetrazol-5-yl)-3-[4′-(trifluoromethoxy)biphenyl-3-yl]-1-oxa-8-azaspiro[4.5]decane-8-carboxamide;
N-(3,4-dimethylisoxazol-5-yl)-3-{3-[5-(trifluoromethyl)pyridin-2-yl]phenyl}-1-oxa-8-azaspiro[4.5]decane-8-carboxamide;
N-(1-methyl-1H-tetrazol-5-yl)-3-{3-[5-(trifluoromethyl)pyridin-2-yl]phenyl}-1-oxa-8-azaspiro[4.5]decane-8-carboxamide;
3-(3-chloro-5-fluorophenyl)-N-(3,4-dimethylisoxazol-5-yl)-3-fluoro-1-oxa-8-azaspiro[4.5]decane-8-carboxamide;
N-1,2-benzisoxazol-3-yl-3-{3-[(5-chloropyridin-2-yl)oxy]phenyl}-1-oxa-8-azaspiro[4.5]decane-8-carboxamide;
or a pharmaceutically acceptable salt thereof.

5. A compound of claim 1 selected from the group of:
3-{3-[(5-bromopyridin-2-yl)oxy]phenyl}-N-pyridin-3-yl-1-oxa-8-azaspiro[4.5]decane-8-carboxamide;
3-{3-[(5-bromopyridin-2-yl)oxy]phenyl}-N-pyridazin-3-yl-1-oxa-8-azaspiro[4.5]decane-8-carboxamide;
3-{3-[(5-chloropyridin-2-yl)oxy]phenyl}-N-pyridazin-3-yl-1-oxa-8-azaspiro[4.5]decane-8-carboxamide;
3-(4′-chlorobiphenyl-3-yl)-N-(3,4-dimethylisoxazol-5-yl)-1-oxa-8-azaspiro[4.5]decane-8-carboxamide;
N-(3,4-dimethylisoxazol-5-yl)-3-[4′-(trifluoromethoxy)biphenyl-3-yl]-1-oxa-8-azaspiro[4.5]decane-8-carboxamide;
N-(3,4-dimethylisoxazol-5-yl)-3-[3-(trifluoromethyl)phenyl]-1-oxa-8-azaspiro[4.5]decane-8-carboxamide;
N-(3,4-dimethylisoxazol-5-yl)-3-{5-[4-(trifluoromethoxy)phenyl]-1,2,4-oxadiazol-3-yl}-1-oxa-8-azaspiro[4.5]decane-8-carboxamide;
N-(1-methyl-1H-tetrazol-5-yl)-3-[3-(trifluoromethoxy)phenyl]-1-oxa-8-azaspiro[4.5]decane-8-carboxamide;
N-(1-methyl-1H-tetrazol-5-yl)-3-[3-(trifluoromethoxy)phenyl]-1-oxa-8-azaspiro[4.5]decane-8-carboxamide, (enantiomer 1);
N-(1-methyl-1H-tetrazol-5-yl)-3-[3-(trifluoromethoxy)phenyl]-1-oxa-8-azaspiro[4.5]decane-8-carboxamide, (enantiomer 2);
3-(3-chlorophenyl)-N-(1-methyl-1H-tetrazol-5-yl)-1-oxa-8-azaspiro[4.5]decane-8-carboxamide;
N-(1-methyl-1H-tetrazol-5-yl)-3-{5-[4-(trifluoromethyl)phenyl]-1,2,4-oxadiazol-3-yl}-1-oxa-8-azaspiro[4.5]decane-8-carboxamide;
N-(1-methyl-1H-tetrazol-5-yl)-3-{5-[4-(trifluoromethoxy)phenyl]-1,2,4-oxadiazol-3-yl}-1-oxa-8-azaspiro[4.5]decane-8-carboxamide;
3-{4-[(3-fluorobenzyl)oxy]phenyl}-N-(1-methyl-1H-tetrazol-5-yl)-1-oxa-8-azaspiro[4.5]decane-8-carboxamide;
3-{4-[(3,4-difluorobenzyl)oxy]phenyl}-N-(1-methyl-1H-tetrazol-5-yl)-1-oxa-8-azaspiro[4.5]decane-8-carboxamide;
N-(1-methyl-1H-tetrazol-5-yl)-3-[3-(trifluoromethyl)phenyl]-1-oxa-8-azaspiro[4.5]decane-8-carboxamide;
N-(1-methyl-1H-tetrazol-5-yl)-3-[4′-(trifluoromethoxy)biphenyl-3-yl]-1-oxa-8-azaspiro[4.5]decane-8-carboxamide;
N-(1-methyl-1H-tetrazol-5-yl)-3-{3-[5-(trifluoromethyl)pyridin-2-yl]phenyl}-1-oxa-8-azaspiro[4.5]decane-8-carboxamide;
or a pharmaceutically acceptable salt thereof.

6. A method of treating pain in a subject, the method comprising administering to a subject in need thereof a therapeutically effective amount of a compound according to claim 1, or a pharmaceutically acceptable salt thereof.

7. A method of treating rheumatoid arthritis in a subject, the method comprising administering to a subject in need thereof a therapeutically effective amount of a compound according to claim 1, or a pharmaceutically acceptable salt thereof.

8. A pharmaceutical composition comprising a therapeutically effective amount of a compound according to claim 1, or a pharmaceutically acceptable salt thereof, and a pharmaceutically acceptable carrier.

9-11. (canceled)

12. The method according to claim 6 wherein the condition is selected from acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.

13-15. (canceled)

16. The compound of N-(1-methyl-1H-tetrazol-5-yl)-3-[3-(trifluoromethoxy)phenyl]-1-oxa-8-azaspiro[4.5]decane-8-carboxamide or a pharmaceutically acceptable salt thereof.

17. A method of treating pain in a subject, the method comprising administering to a subject in need thereof a therapeutically effective amount of a compound according to claim 16, or a pharmaceutically acceptable salt thereof.

18. A pharmaceutical composition comprising the compound of claim 16 or a pharmaceutically acceptable salt thereof.
